(19) United States
(12) Patent Application Publication
(10) Pub. No.: US 2017/0071208 A1
(43) Pub. Date: Mar. 16, 2017

(54) COMPOSITIONS FOR MOSQUITO CONTROL AND USES OF SAME

(71) Applicant: Forrest Innovations Ltd., Caesarea (IL)

(72) Inventors: Nitzan PALDI, Moshav Bar Giora (IL); Humberto Freire BONCRISTIANI JUNIOR, Odenton, MD (US); Eyal MAORI, Rishon-LeZion (IL); Avital WEISS, Karkur (IL); Emerson Soares BERNARDES, Sao Paulo (BR)

(21) Appl. No.: 15/308,394

(22) PCT Filed: May 4, 2015
(86) PCT No.: PCT/IL2015/050468

§ 371 Date: Nov. 2, 2016

Related U.S. Application Data
(60) Provisional application No. 61/988,235, filed on May 4, 2014.
(60) Provisional application No. 61/988,246, filed on May 4, 2014.
(60) Provisional application No. 61/988,237, filed on May 4, 2014.
(60) Provisional application No. 61/988,236, filed on May 4, 2014.
(60) Provisional application No. 61/988,234, filed on May 4, 2014.

(51) Int. Cl. A01N 57/16; A01N 25/10; C12N 15/113

(52) U.S. Cl. CPC A01N 57/16; C12N 15/113; A01N 25/10

(57) ABSTRACT

A composition-of-matter for mosquito control is provided. The composition comprises a cell which comprises an exogenous naked dsRNA which specifically down-regulates expression of a gene being endogenous to a mosquito or which specifically down-regulated expression of a gene being endogenous to a mosquito pathogen. Further provided is a composition-of-matter for mosquito control, comprising a cell comprising a nucleic acid larvicide. Also provided are methods of producing and using the compositions.

### FIELD AND BACKGROUND OF THE INVENTION

The present invention, in some embodiments thereof, relates to compositions for mosquito control and uses of same.

Mosquitoes are the major vectors for a number of human and animal diseases, including malaria, yellow fever and dengue fever. Over 1 million people die from mosquito-borne diseases every year, and hundreds of millions more experience pain and suffering from illnesses transmitted by mosquitoes.

There is neither specific medication nor vaccine for Dengue. The only way currently to control the disease is to control the mosquito, Aedes aegypti, which spreads the disease. There is no cure for yellow fever but there is a vaccine; however it is expensive and not available to protect other parts of the world. There is no currently available drug regimen guarantees 100% protection against Malaria, and prevention of infection requires taking antimalarial medication as directed in addition to prevention of mosquito bites. Antimalarials do not actually prevent the disease but only act in the bloodstream to suppress clinical symptoms by inhibiting parasite development in red blood cells.

In order to prevent human disease caused by the viruses and parasites mentioned above, a systematic mosquito surveillance system is required. Nowadays, it is accepted that the success of such actions depends on the implementation of an integrated mosquito management program (IMM).

The aim of these programs is to optimize the control of mosquitoes in an economical and environmentally friendly way. Specifically, Integrated Mosquito Management is a comprehensive mosquito prevention/control strategy that utilizes all available mosquito control methods singly or in combination to exploit the known vulnerabilities of mosquitoes in order to reduce their numbers to tolerable levels while maintaining a quality environment. IMM does not emphasize mosquito elimination or eradication. Integrated mosquito management methods are specifically tailored to safely counter each stage of the mosquito life cycle. Prudent mosquito management practices for the control of immature mosquitoes (larvae and pupae) include such methods as the use of biological controls (native, noninvasive predators), source reduction (water or vegetation management or other compatible land management uses), water sanitation practices as well as the use of registered larvicides. When source elimination or larval control measures are not feasible or are clearly inadequate, or when faced with imminent mosquito-borne disease, application of registered adulticides may be needed. However, larvicides/adulticides efficacy is now threatened by the rise of resistance in target populations. Such phenomenon is occurring worldwide in all major disease vector mosquito species and spreads at a rapid rate [Harris et al. (2010) Am. J. Trop. Med. Hyg. 83, 277e284; Marcombe et al. (2009a) Am. J. Trop. Med. Hyg. 80, 745e751; Marcombe et al. (2009b) BMC Genomics 10, 494; Ranson et al. (2009) Malar. J. 8, 299].

Larviciding is an ecologically safe preventive method used to interrupt the development of larvae or pupa into adult mosquitoes. Larviciding is also a general term for killing immature mosquitoes by applying agents, collectively called larvicides, to control mosquito larvae and/or pupae. Larvicides may be grouped into two broad categories: biorational pesticides (biopesticides) and conventional, broad-spectrum chemical pesticides.

Biochemical agents such as Insect Growth Regulators (IGRS) controls insects by interrupting their life cycle, rather than through direct toxicity. Based on this mode of action, the U.S. Environmental Protection Agency (EPA) considers it to be a biochemical pesticide. The IGRS mimics naturally occurring insect biochemicals that are responsible for insect development. Through the mimicry, IGRS keeps the mosquito larvae from developing into adults that would emerge from the pupae. It is able to exert this effect at very small concentrations. The first IGRS, which contained several methoprene isomers, was registered in 1975 [Henrick, (2007) Methoprene. In: Floore, T.G. (Ed.). Biorational Control of Mosquitoes. Bulletin of the American Mosquito Control Association No. 7. St Louis, Mo.: Allen Press]. Methoprene products currently are the only IGRS registered for use in the USA. Methoprene is a juvenile hormone (JH) analog, which mimicries the natural hormone from insects. JH is involved in the regulation of physiological processes in insects including mating and metamorphosis. Therefore, these chemicals interfere with normal insect growth and maturation and induce abnormal larval growth patterns.

Resistance has been defined as ‘the developed ability in a strain of insects to tolerate doses of toxicants that would prove lethal to the majority of individuals in a normal population of the same species’ [Clark & Yamaguchi, (2002) Scope and Status of Pesticide Resistance. In Agrochemical Resistance: Extent, Mechanism and Detection, eds. J. M Clark & I. Yamaguchi, pp 1-22. Washington, D.C.: American Chemical Society]. In a susceptible population, individuals with resistant genes to a given insecticide are rare, and usually range between 10⁻⁵ and 10⁻⁸ in number, but widespread use of a toxicant favors the prevalence of the resistant individuals. These individuals multiply fast in the absence of intraspecific competition and, over a number of generations, quickly become the dominant proportion of the population. Hence, the insecticide is no longer effective and the insects are considered to be resistant.

In addition to pesticides and insecticides, chemicals commonly used in agriculture also include fertilizers, herbicides, fungicides and various adjuvants that increase their efficiency. Although these compounds are usually non-toxic to insects, their presence in breeding sites has been shown to affect tolerance to insecticides via the modulation of their detoxification system. For instance, Chironomus tentans larvae exposed to the herbicide alachlor respond by enhanced GST activities [Li et al. (2009) Insect Biochem. Mol. Biol., 39, 745e754]. Ae. albopictus larvae exposed for 48 h to the fungicides triadimefon, diniconazole and pentachlorophenol showed an increased tolerance to carbaryl [Suwanchaichinda and Brattsten, (2001) Pestic. Biochem. Physiol., 70, 63e73]. The strong effect observed with pentachlorophenol was further linked to a strong induction of P450s. Poupardin et al. [(2008) Insect Biochem. Mol. Biol. 38, 540e551; (2010) Insect Mol. Biol., 19, 185e193] demonstrated that exposing Ae. aegypti larvae to a sub-lethal dose of copper sulphate, frequently used in agriculture as a fungicide, enhance their tolerance to the pyrethroid permethrin. This effect was correlated to an elevation of P450 activities and the induction of CYP genes preferentially transcribed in detoxification tissues and showing high homology to known pyrethroid metabolizers. Similarly, exposing Ae. Aegypti larvae to the herbicide glyphosate, the active molecule of Roundup, led to a significant increase of their tolerance to permethrin together with the induction of multiple detoxification genes [(Riaz et al. (2009) Aquat. Toxicol., 93, 61e69].

Mosquito resistance has also been described against biolarvicides. Specifically, the development of resistance in Culex quinquefasciatus to the Biopesticide Bacillus sphaericus (B.s.) has been noted by Rodcharoen et al., Journal of Economic Entomology, Vol. 87, No. 5, 1994, pp. 1133-1140. In addition, resistance to methoprene was soon demonstrated in several species [Dyte, (1972) Nature, 238(5358):48-9; Cerf & Georghiou, (1972) Nature, 239(5372):401-2].

One method of introducing dsRNA to the larvae is by dehydration. Specifically, larvae are dehydrated in a NaCl solution and then rehydrated in water containing double-stranded RNA. This process is suggested to induce gene silencing in mosquito larvae.

### SUMMARY OF THE INVENTION

According to an aspect of some embodiments of the present invention there is provided a composition-of-matter for mosquito control, comprising a cell comprising an exogenous naked dsRNA which specifically down-regulates expression of a gene being endogenous to a mosquito or which specifically down-regulated expression of a gene being endogenous to a mosquito pathogen.

According to an aspect of some embodiments of the present invention there is provided a composition-of-matter for mosquito control, comprising a cell comprising a nucleic acid larvicide.

According to an aspect of some embodiments of the present invention there is provided a composition-of-matter for mosquito control, comprising a cell comprising a nucleic acid larvicide affecting fertility or fecundity of a female mosquito.

According to an aspect of some embodiments of the present invention there is provided a composition-of-matter for mosquito control comprising a nucleic acid larvicide that targets a piRNA pathway gene and/or a sterility gene.

According to an aspect of some embodiments of the present invention there is provided a composition-of-matter for mosquito control comprising a nucleic acid larvicide that targets a gene comprising Aub (AAEL007698) and Argonaute-3 (AAEL007823).

According to some embodiments of the invention, the nucleic acid larvicide comprises at least one dsRNA.

According to some embodiments of the invention, the composition-of-matter comprises a dsRNA which comprises SEQ ID NO: 1858 and a dsRNA which comprises SEQ ID NO: 1823.

According to an aspect of some embodiments of the present invention there is provided a method of producing a larvicidal composition, the method comprising introducing into a cell a nucleic acid larvicide, thereby producing the larvicide.

According to an aspect of some embodiments of the present invention there is provided a method of producing a larvicidal composition, the method comprising introducing into a cell a nucleic acid larvicide affecting fertility or fecundity of a female mosquito, thereby producing the larvicide.

According to some embodiments of the invention, the introducing is effected by electroporation.

According to some embodiments of the invention, the introducing is effected by particle bombardment.

According to some embodiments of the invention, the introducing is effected by chemical-based transfection.

According to some embodiments of the invention, the nucleic acid larvicide down-regulates a target gene selected from the group consisting of:

(i) affecting larval survival;

(ii) interfering with metamorphosis of larval stage to adulthood;

(iii) affecting susceptibility of mosquito larvae to a larvicide;

(iv) affecting susceptibility of an adult mosquito to an adulticide/insecticide; and

(v) affecting fertility or fecundity of a male or female mosquito.

According to some embodiments of the invention, the target gene is selected from the group consisting of 1-427, 430-1813, 1826-1832.

According to some embodiments of the invention, the target gene is selected from the group consisting of P-glycoprotein (AAEL010379), Argonaute-3 (AAEL007823), Cytochrome p450 (CYP9J26), Sodium channel (AAEL008297), Aub (AAEL007698), AeSCP-2 (AF510492.1), AeAct-4 (AY531222.2), AAEL002000, AAEL005747, AAEL005656, AAEL017015, AAEL005212, AAEL005922, AAEL000903 and AAEL005049.

According to some embodiments of the invention, the target gene comprises Aub (AAEL007698) and Argonaute-3 (AAEL007823).

According to some embodiments of the invention, the nucleic acid larvicide which down-regulates the target gene is a dsRNA.

According to some embodiments of the invention, the dsRNA comprises SEQ ID NOs: 1858 and 1823.

According to some embodiments of the invention, the cell is an algal cell.

According to some embodiments of the invention, the cell is a microbial cell.

According to some embodiments of the invention, the cell is a bacterial cell.

According to some embodiments of the invention, the composition further comprises a food-bait.

According to some embodiments of the invention, the composition is formulated in a formulation selected from the group consisting of technical powder, wettable powder, dust, pellet, briquette, tablet and granule.

According to some embodiments of the invention, the granule is selected from the group consisting of an impregnated granule, dry flowable, wettable granule and water dispersible granule.

According to some embodiments of the invention, the composition is formulated as a non-aqueous or aqueous suspension concentrate.

According to some embodiments of the invention, the composition is formulated as a semi-solid form.

According to some embodiments of the invention, the semi-solid form comprises an agarose.

According to some embodiments of the invention, the cell is lyophilized.

According to some embodiments of the invention, the cell is non-transgenic.

According to some embodiments of the invention, the composition-of-matter or method further comprises an RNA-binding protein.

According to some embodiments of the invention, the nucleic acid larvicide comprises a dsRNA.

According to some embodiments of the invention, the dsRNA is a naked dsRNA.

According to some embodiments of the invention, the dsRNA comprises a carrier.

According to some embodiments of the invention, the carrier comprises a polyethyleneimine (PEI).

According to some embodiments of the invention, the dsRNA is effected at a dose of 0.001-1 μg/μL for soaking or at a dose of 1 pg to 10 μg/larvae for feeding.

According to some embodiments of the invention, the dsRNA is selected from the group consisting of SEQ ID NOs: 1822-1825 and 1857-1868.

According to some embodiments of the invention, the dsRNA is selected from the group consisting of siRNA, shRNA and miRNA.

According to some embodiments of the invention, the cell is devoid of a heterologous promoter for driving expression of the dsRNA in the plant.

According to some embodiments of the invention, the nucleic acid larvicide is greater than 15 base pairs in length.

According to some embodiments of the invention, the nucleic acid larvicide is 19 to 25 base pairs in length.

According to some embodiments of the invention, the nucleic acid larvicide is 30-100 base pairs in length.

According to some embodiments of the invention, the nucleic acid larvicide is 100-800 base pairs in length.

According to some embodiments of the invention, the composition further comprises at least one of a surface-active agent, an inert carrier vehicle, a preservative, a humectant, a feeding stimulant, an attractant, an encapsulating agent, a binder, an emulsifier, a dye, an ultra-violet protector, a buffer, a flow agent or fertilizer, micronutrient donors, or other preparations that influence the growth of the plant.

According to some embodiments of the invention, the composition of matter has an inferior impact on an adult mosquito as compared to the larvae.

According to some embodiments of the invention, the composition further comprises a chemical larvicide or a biochemical larvicide or a combination of same.

According to some embodiments of the invention, the larvicide is selected from the group consisting of Temephos, Diflubenzuron, methoprene, Bacillus sphaericus, and Bacillus thuringiensis israelensis.

According to some embodiments of the invention, the larvicide comprises an adulticide.

According to some embodiments of the invention, the adulticide is selected from the group consisting of deltamethrin, malathion, naled, chlorpyrifos, permethrin, resmethrin and sumithrin.

According to an aspect of some embodiments of the present invention there is provided a method of controlling or exterminating mosquitoes, the method comprising feeding larvae of the mosquitoes with an effective amount of the composition-of-matter of some embodiments of the invention, thereby controlling or exterminating the mosquitoes.

According to some embodiments of the invention, the mosquitoes comprise female mosquitoes capable of transmitting a disease to a mammalian organism.

According to some embodiments of the invention, the mosquitoes are of a species selected from the group consisting of Aedes aegypti and Anopheles gambiae.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart illustration depicting introduction of dsRNA into mosquito larvae via soaking with “naked” dsRNA. In short, third instar larvae were treated (in groups of 100 larvae) in a final volume of 3 mL of dsRNA solution in autoclaved water with 0.5 μg/μL dsRNA. The control group was kept in 3 ml sterile water only. Larvae were soaked in the dsRNA solutions for 24 hr at 27° C., and then transferred into new containers (300 larvae/1500 mL of chlorine-free tap water), which were also maintained at 27° C., and were provided with lab dog/cat diet (Purina Mills) suspended in water as a source of food on a daily basis. As pupae developed, they were transferred to individual vials to await eclosion and sex sorting. For bioassays purpose only females up to five days old were used. Then, mosquitoes were subjected to pyrethroid adulticide assay.

FIG. 2 is a flowchart illustration depicting introduction of dsRNA into mosquito larvae via soaking with “naked” dsRNA plus additional larvae feeding with food-containing dsRNA. After soaking in the dsRNA solutions for 24 hr at 27° C. (as indicated in FIG. 1 above), the larvae were transferred into new containers (300 larvae/1500 mL of chlorine-free tap water), and were provided agarose cubes containing 300 μg of dsRNA once a day for a total of four days. The larvae were reared until adult stage. For bioassays purpose only females up to five days old are used. Then, mosquitoes were subjected to pyrethroid adulticide assay.

FIG. 3 is a flowchart illustration depicting introduction of dsRNA into mosquito larvae via feeding with food-containing dsRNA only. Third instar larvae were fed (in groups of 300 larvae) in a final volume of 1500 mL of chlorine-free tap water with agarose cubes containing 300 μg of dsRNA once a day for a total of four days. The larvae were reared until adult stage. For bioassays purpose only females up to five days old are used. Then, mosquitoes were subjected to pyrethroid adulticide assay.

FIG. 4 is a flowchart illustration depicting dsRNA production.

FIGS. 5A-C are graphs illustrating the dose-response curves for 3- to 5-day-old Aedes aegypti female mosquitoes on insecticide-susceptible Rockefeller strain (FIG. 5A) and on insecticide-resistant Rio de Janeiro strain (FIG. 5B). Mosquitoes were exposed to different concentrations of deltamethrin in 250-mL glass bottles for up to 24 hours and the percentage of mortality for each time point is shown. FIG. 5C, comparison of the mortality rates of female mosquitoes from Rockefeller (Rock) and Rio de Janeiro (RJ) strains exposed to 2 μg/mL of deltamethrin for different time-points. Data represent mean values of three replicates with standard deviation.

FIGS. 6A-B are photographs illustrating allele specific PCR for genotyping kdr mutations in the Aedes aegypti Rio de Janeiro strain. FIGS. 6A-B represent reactions for the 1016 and 1534 mutation sites, respectively. Amplicons were resolved in a 10% polyacrylamide gel electrophoresis and stained with Gel Red. FIG. 6A, amplicons of approximately 80 and 100 bp correspond to alleles 1016 Val⁺ and 1016 Ile^{kdr}, respectively. FIG. 6B, amplicons of 90 and 110 bp correspond to alleles 1534 Phe⁺ and 1534 Cys^{kdr}, respectively. Rockefeller Ae. aegypti mosquito strain was used as positive homozygous dominant control for both mutation sites. C−: negative control.

FIGS. 7A-C are graphs illustrating that sodium channel gene silencing on Ae. aegypti mosquitoes (RJ strain) results in increased susceptibility to Pyrethroid adulticide. FIG. 7A, larvae from Ae. aegypti RJ strain (3^{rd} instar) were soaked for 24 hours in 0.5 μg/μL of sodium channel dsRNA or only in water, and then reared until adult stage. Adult females were exposed to deltamethrin (0.5 μg/bottle) for different time-points, as indicated, and mortality rates for each time point is shown. Data show the mean±standard deviation of four replicates, and is representative of 3 independent experiments. FIG. 7B, adult mosquitoes (males and females) previously soaked with sodium channel dsRNA or only water were collected before the treatment with deltamethrin and analyzed for sodium channel mRNA expression using qPCR method. FIG. 7C, live and immediately dead female mosquitoes were collected after exposure to deltamethrin and the mRNA expression of sodium channel was determined by qPCR analysis. ***p<0.0001; ****p<0.00001.

FIG. 8 is a graph illustrating that sodium channel gene silencing on A. aegypti mosquitoes (RJ strain) results in increased susceptibility to Pyrethroid adulticide. Larvae from Ae. aegypti RJ strain (3^{rd} instar) were soaked for 24 hours in 0.5 μg/μL of sodium channel dsRNA or only in water, and then were fed 4 times with food plus agarose 2% containing dsRNA until they reach pupa stage. After emergence, adult females were exposed to deltamethrin (0.5 μg/bottle) for different time-points, as indicated, and mortality rates for each time point is shown. Data show the mean±standard deviation of four replicates, and is representative of 3 independent experiments. *p<0.01; ***p<0.0001.

FIG. 9 is a graph illustrating that feeding CYP9J29 dsRNA to larvae affects the susceptibility of adult Ae. aegypti mosquitoes to Pyrethroid adulticide. Larvae from A. aegypti RJ strain (3^{rd} instar) were soaked for 24 hours in 0.1 μg/μL of target #3 (CYP9J26) dsRNA or only in water; and then were fed 4 times with food plus agarose 2% containing dsRNA until they reach pupa stage. Adult females were exposed to deltamethrin (0.5 μg/bottle) for different time-points, as indicated, and then percentage of mortality for each time point is shown. Data represent the mean±standard deviation of four replicates. **p<0.001.

FIGS. 10A-C are graphs illustrating gene silencing in A. aegypti larvae. 3^{rd} instar larvae from Ae. aegypti were soaked for 24 hours in 0.5 μg/mL of (FIG. 10A) P-glycoprotein (PgP); (FIG. 10B) Ago-3 or (FIG. 10C) sodium channel dsRNA. Larvae soaked only in water were used as control. At 6, 24 and 48 hours after the end of dsRNA treatment, larvae were collected and analysed for PgP, Ago-3 and Sodium channel mRNA expression by qPCR. Data represent the mean±standard deviation of four replicates. *p<0.01 **p<0.001; ***p<0.0001; ****p<0.00001.

FIGS. 11A-B are graphs illustrating P-glycoprotein and Ago-3 expression in Ae. aegypti adult mosquitoes soaked with dsRNA. Third instar larvae from Ae. aegypti were soaked for 24 hours in 0.5 μg/mL of (FIG. 11A) P-glycoprotein (PgP) and (FIG. 11B) Ago-3, and then reared until adult stage. Adult mosquitoes (males and females) previously soaked with the indicated dsRNA or only water were collected and analyzed for PgP and Ago-3 mRNA expression using qPCR method. Data represent the mean±standard deviation of five replicates. **p<0.001.

FIG. 12 is a flowchart illustration depicting introduction of dsRNA into mosquito larvae via soaking with different doses of “naked” dsRNA plus additional larvae feeding with food-containing dsRNA. Step a) 100 larvae from A. aegypti Rockefeller strain (3^{rd} instar) were soaked for 24 hours with the respective dsRNAs (concentration range from 0.02-0.5 μg/μL) or only in water and were then fed 2 times with food plus agarose 2% containing dsRNA until they reach adult stage (Step b). Step c) The adults arising were allowed to copulate for 3-5 days. Step d) mosquitoes were fed with defibrinated sheep blood. Step e) after blood feeding 15 fully-engorged females were transferred into 3 small cages to be assayed for oviposition. Step f) the total number of laid eggs and the percentage of hatched eggs were counted. FIGS. 13A-B are graphs illustrating larvae from Ae. aegypti Rockefeller strain (3^{rd} instar) soaked for 24 hours in 0.5 μg/μL of Aubergine (Aub) or Argonaute-3 (Ago) dsRNAs or water only. After soaking, larvae were separated in 3 different cages (containing 100 larvae each) and were treated twice with agarose plug containing dsRNA. The adults arising were allowed to copulate for 3-5 days and then fed with defibrinated sheep blood. After blood feeding 15 fully-engorged females were transferred into small cages to be assayed for oviposition. (FIG. 13A) The total number of laid eggs and the percentage of hatched eggs were counted (FIG. 13B).

FIGS. 14A-B are graphs illustrating larvae from Ae. aegypti Rockefeller strain (3^{rd} instar) soaked for 24 hours in 0.02 μg/μL of AeAct-4 dsRNA or water only. After soaking, larvae were separated in 3 different cages (containing 100 larvae each) and were treated twice with agarose plug containing dsRNA. The adults arising were allowed to copulate for 3-5 days and then fed with defibrinated sheep blood. After blood feeding 15 fully-engorged females were transferred into small cages to be assayed for oviposition. (FIG. 14A) The total number of laid eggs and the percentage of hatched eggs were counted (FIG. 14B).

FIGS. 15A-B are graphs illustrating Larvae from A. aegypti Rockefeller strain (3^{rd} instar) soaked for 24 hours in 0.05 μg/μL of AAEL005922 dsRNA or 0.06 μg/μL of AAEL000903 dsRNA or water only. After soaking, larvae were separated in 3 different cages (containing 100 larvae each) and were treated twice with agarose plug containing dsRNA. The adults arising were allowed to copulate for 3-5 days and then fed with defibrinated sheep blood. After blood feeding 15 fully-engorged females were transferred into small cages to be assayed for oviposition. (FIG. 15A) The total number of laid eggs and the percentage of hatched eggs were counted (FIG. 15B).

FIGS. 16A-B are graphs illustrating larvae from Ae. aegypti Rockefeller strain (3^{rd} instar) soaked for 24 hours in 0.06 μg/μL of AAEL017015 dsRNA, or 0.06 μg/μL of AAEL005212 dsRNA, 0.5 μg/μL of Aubergine (Aub)+Argonaute-3 (Ago) dsRNA or water only. After soaking, larvae were separated in 3 different cages (containing 100 larvae each) and treated twice with agarose plug containing dsRNA. The adults arising were allowed to copulate for 3-5 days and then fed with defibrinated sheep blood. After blood feeding 15 fully-engorged females were transferred into small cages to be assayed for oviposition. (FIG. 16A) The total number of laid eggs and the percentage of hatched eggs were counted (FIG. 16B).

### DESCRIPTION OF SPECIFIC EMBODIMENTS OF THE INVENTION

The present invention, in some embodiments thereof, relates to compositions for mosquito control and uses of same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NO: 1822 is expressed in a DNA sequence format (e.g., reciting T for thymine), but it can refer to either a DNA sequence that corresponds to an endo 1,4 beta gluconase nucleic acid sequence, or the RNA sequence of an RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

While reducing the present invention to practice, the present inventors have uncovered that feeding dsRNA to mosquito larvae is an effective method for silencing gene expression in adult mosquitoes.

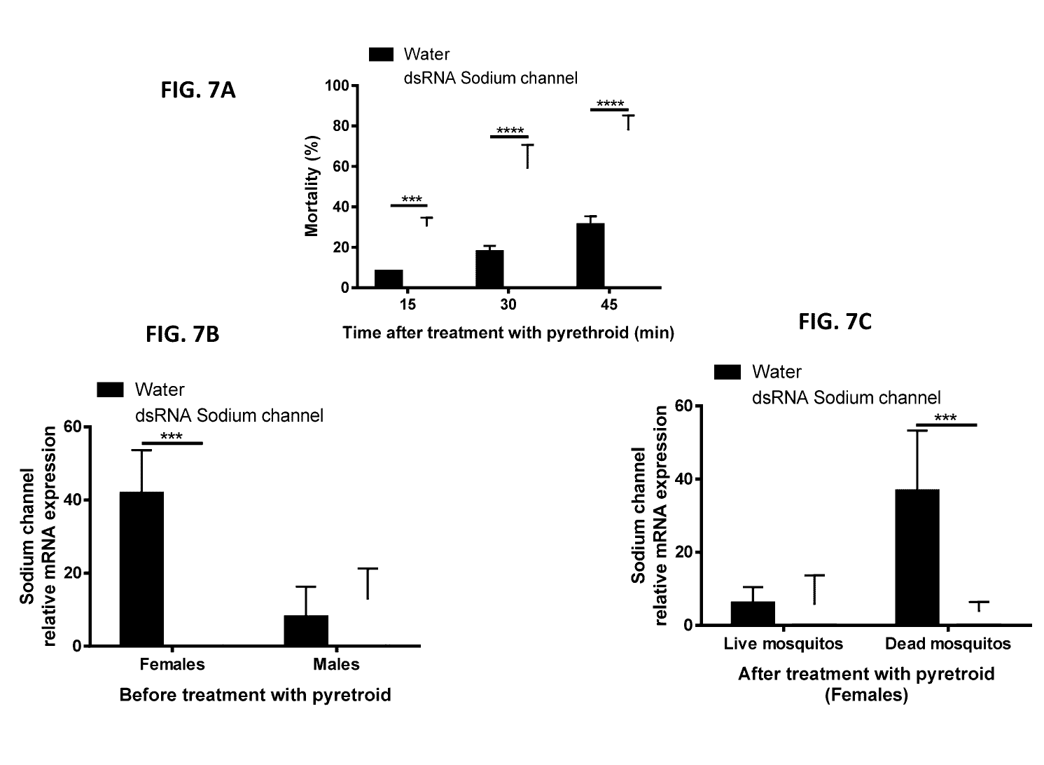

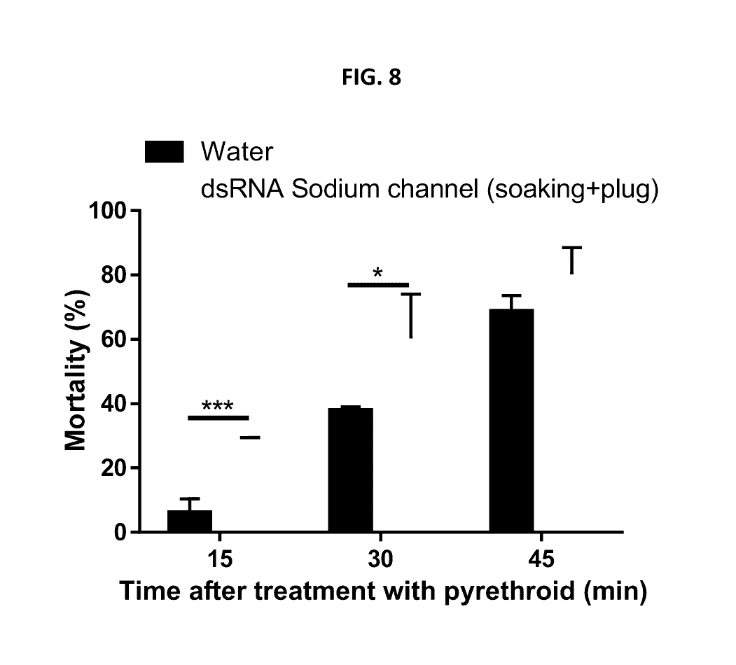

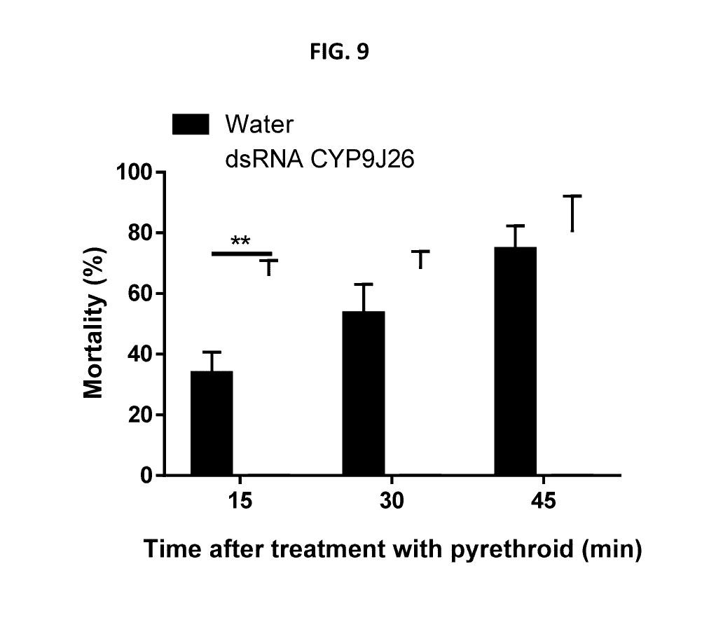

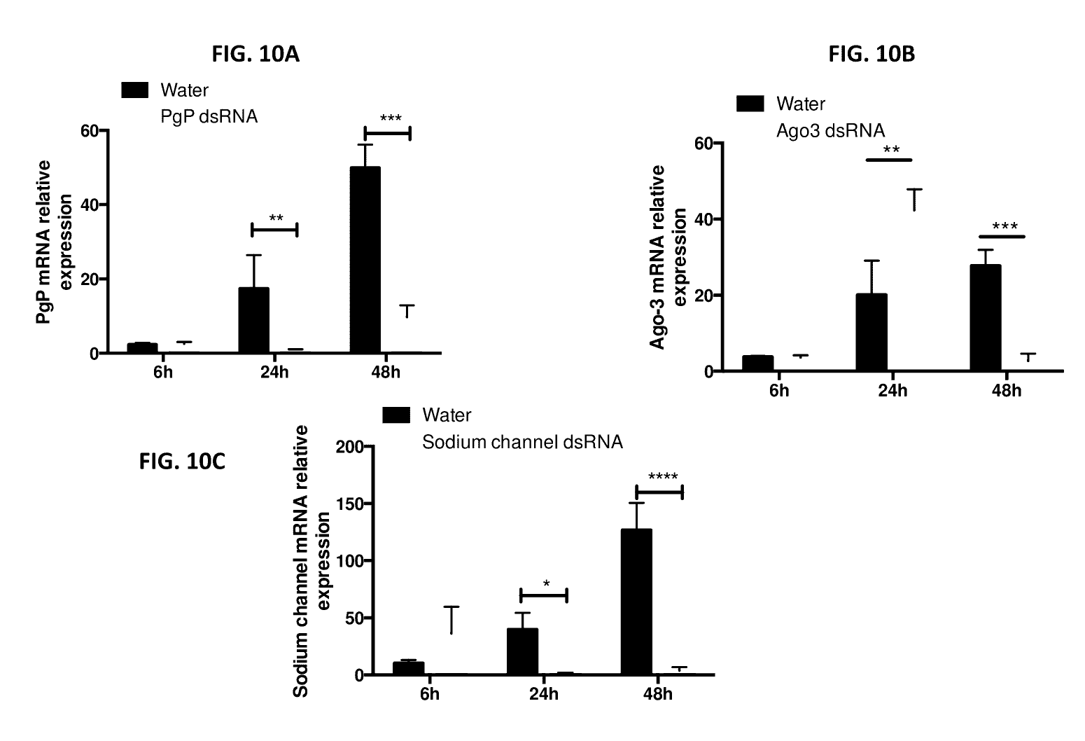

Specifically, the present inventors have shown that feeding mosquito larvae with dsRNA targeting specific genes for two to four days (via agarose cubes, until they reach pupa stage) with or without previous soaking with dsRNA for 24 hours (e.g. sodium channel, PgP, ago-3 and Cytochrome p450) efficiently decreases gene expression (FIGS. 10A-C) and results in higher susceptibility (FIGS. 8, 9) in adult mosquitoes. Importantly, female mosquitoes showed a decreased expression in the mRNA level for sodium channel before deltamethrin treatment (FIG. 7B) and dead female mosquitoes previously treated with dsRNA showed a striking decrease in mRNA expression level for sodium channel (FIG. 7C). Furthermore, it was illustrated that feeding mosquito larvae with dsRNA significantly reduced the number of hatchings of eggs of adult female mosquitoes (FIGS. 13A-B, 14A-B, 15A-B and 16A-B).

According to an aspect of the invention there is provided a composition-of-matter for mosquito control, comprising a cell comprising an exogenous naked dsRNA which specifically down-regulates expression of a gene being endogenous to a mosquito or which specifically down-regulated expression of a gene being endogenous to a mosquito pathogen.

As used herein the term “exogenous” refers to an externally added nucleic acid molecule which is not naturally occurring in the cell.

According to an aspect of the invention there is provided a composition-of-matter for mosquito control, comprising a cell which comprises a nucleic acid larvicide.

According to another aspect of the invention there is provided a composition-of-matter for mosquito control, comprising a cell comprising a nucleic acid larvicide affecting fertility or fecundity of a female mosquito.

The term “mosquito” or “mosquitoes” as used herein refers to an insect of the family Culicidae. The mosquito of the invention may include an adult mosquito, a mosquito larva, a pupa or an egg thereof.

An adult mosquito is defined as any of slender, long-legged insect that has long proboscis and scales on most parts of the body. The adult females of many species of mosquitoes are blood-eating pests. In feeding on blood, adult female mosquitoes transmit harmful diseases to humans and other mammals.

A mosquito larvae is defined as any of an aquatic insect which does not comprise legs, comprises a distinct head bearing mouth brushes and antennae, a bulbous thorax that is wider than the head and abdomen, a posterior anal papillae and either a pair of respiratory openings (in the subfamily Anophelinae) or an elongate siphon (in the subfamily Culicinae) borne near the end of the abdomen.

Typically, a mosquito's life cycle includes four separate and distinct stages: egg, larva, pupa, and adult. Thus, a mosquito's life cycle begins when eggs are laid on a water surface (e.g. Culex, Culiseta, and Anopheles species) or on damp soil that is flooded by water (e.g. Aedes species). Most eggs hatch into larvae within 48 hours. The larvae live in the water feeding on microorganisms and organic matter and come to the surface to breathe. They shed their skin four times growing larger after each molting and on the fourth molt the larva changes into a pupa. The pupal stage is a resting, non-feeding stage of about two days. At this time the mosquito turns into an adult. When development is complete, the pupal skin splits and the mosquito emerges as an adult.

According to one embodiment, the mosquitoes are of the sub-families Anophelinae and Culicinae. According to one embodiment, the mosquitoes are of the genus Culex, Culiseta, Anopheles and Aedes. Exemplary mosquitoes include, but are not limited to, Aedes species e.g. Aedes aegypti, Aedes albopictus, Aedes polynesiensis, Aedes australis, Aedes cantator, Aedes cinereus, Aedes rusticus, Aedes vexans; Anopheles species e.g. Anopheles gambiae, Anopheles freeborni, Anopheles arabiensis, Anopheles funestus, Anopheles gambiae Anopheles moucheti, Anopheles balabacensis, Anopheles baimaii, Anopheles culicifacies, Anopheles dirus, Anopheles latens, Anopheles leucosphyrus, Anopheles maculatus, Anopheles minimus, Anopheles fluviatilis s.l., Anopheles sundaicus Anopheles superpictus, Anopheles farauti, Anopheles punctulatus, Anopheles sergentii, Anopheles stephensi, Anopheles sinensis, Anopheles atroparvus, Anopheles pseudopunctipennis, Anopheles bellator and Anopheles cruzii; Culex species e.g. C. annulirostris, C. antennatus, C. jenseni, C. pipiens, C. pusillus, C. quinquefasciatus, C. rajah, C. restuans, C. salinarius, C. tarsalis, C. territans, C. theileri and C. tritaeniorhynchus; and Culiseta species e.g. Culiseta incidens, Culiseta impatiens, Culiseta inornata and Culiseta particeps.

According to one embodiment, the mosquitoes are capable of transmitting disease-causing pathogens. The pathogens transmitted by mosquitoes include viruses, protozoa, worms and bacteria.

Non-limiting examples of viral pathogens which may be transmitted by mosquitoes include the arbovirus pathogens such as Alphaviruses pathogens (e.g. Eastern Equine encephalitis virus, Western Equine encephalitis virus, Venezuelan Equine encephalitis virus, Ross River virus, Sindbis Virus and Chikungunya virus), Flavivirus pathogens (e.g. Japanese Encephalitis virus, Murray Valley Encephalitis virus, West Nile Fever virus, Yellow Fever virus, Dengue Fever virus, St. Louis encephalitis virus, and Tick-borne encephalitis virus), Bunyavirus pathogens (e.g. La Crosse Encephalitis virus, Rift Valley Fever virus, and Colorado Tick Fever virus) and Orbivirus (e.g. Bluetongue disease virus).

Non-limiting examples of worm pathogens which may be transmitted by mosquitoes include nematodes e.g. filarial nematodes such as Wuchereria bancrofti, Brugia malayi, Brugia pahangi, Brugia timori and heartworm (Dirofilaria immitis)).

Non-limiting examples of bacterial pathogens which may be transmitted by mosquitoes include gram negative and gram positive bacteria including Yersinia pestis, Borellia spp, Rickettsia spp, and Erwinia carotovora.

Non-limiting examples of protozoa pathogens which may be transmitted by mosquitoes include the Malaria parasite of the genus Plasmodium e.g. Plasmodium falciparum, Plasmodium vivax, Plasmodium ovale, Plasmodium malariae, Plasmodium berghei, Plasmodium gallinaceum, and Plasmodium knowlesi.

According to one embodiment, the mosquito comprises a female mosquito being capable of transmitting a disease to a mammalian organism.

Non-limiting examples of mosquitoes and the pathogens which they transmit include species of the genus Anopheles (e.g. Anopheles gambiae) which transmit malaria parasites as well as microfilariae, arboviruses (including encephalitis viruses) and some species also transmit Wuchereria bancrofti; species of the genus Culex (e.g. C. pipiens) which transmit West Nile virus, filariasis, Japanese encephalitis, St. Louis encephalitis and avian malaria; species of the genus Aedes (e.g. Aedes aegypti, Aedes albopictus and Aedes polynesiensis) which transmit nematode worm pathogens (e.g. heartworm (Dirofilaria immitis)), arbovirus pathogens such as Alphaviruses pathogens that cause diseases such as Eastern Equine encephalitis, Western Equine encephalitis, Venezuelan equine encephalitis and Chikungunya disease; Flavivirus pathogens that cause diseases such as Japanese encephalitis, Murray Valley Encephalitis, West Nile fever, Yellow fever, Dengue fever, and Bunyavirus pathogens that cause diseases such as LaCrosse encephalitis, Rift Valley Fever, and Colorado tick fever.

According to one embodiment, pathogens that may be transmitted by Aedes aegypti are Dengue virus, Yellow fever virus, Chikungunya virus and heartworm (Dirofilaria immitis).

According to one embodiment, pathogens that may be transmitted by Aedes albopictus include West Nile Virus, Yellow Fever virus, St. Louis Encephalitis virus, Dengue virus, and Chikungunya fever virus.

According to one embodiment, pathogens that may be transmitted by Anopheles gambiae include malaria parasites of the genus Plasmodium such as, but not limited to, Plasmodium falciparum, Plasmodium vivax, Plasmodium ovale, Plasmodium malariae, Plasmodium berghei, Plasmodium gallinaceum, and Plasmodium knowlesi.

As used herein the phrase “mosquito control” refers to managing the population of mosquitoes to reduce their damage to human health, economies, and enjoyment. According to some embodiments of the invention, mosquito management is typically effected using larvicidally effective compositions and compositions having mosquito “aversion activity” which causes a mosquito to avoid deleterious behavior such as a mosquito biting.

As used herein, the term “larvicidal” or “larvicidal activity” refers to the ability of interfering with a mosquito life cycle resulting in an overall reduction in the mosquito population. The larvicidal composition acts (down-regulates gene expression) at the larval stage. The activity of the larvicidal composition may be manifested immediately (e.g., by affecting larval survival) or only at later stages, as described below. For example, the term larvicidal includes inhibition of a mosquito from progressing from one form to a more mature form, e.g., transition between various larval instars or transition from larva to pupa or pupa to adult. Alternatively or additionally, the term larvicidal affects mosquito fertility or fecundity. Hence the down-regulation of the target gene may induce male or female sterility. Further, the term “larvicidal” is intended to encompass, for example, anti-mosquito activity during all phases of a mosquito life cycle; thus, for example, the term includes larvacidal, ovicidal, and adulticidal activity. According to a specific embodiment all of which stem from the activity at the larval stage. Alternatively or additionally, larvicide encompasses both “larva-specific” larvicides, and non-specific larvicides.”

According to one embodiment the larvicide may affect fertility or fecundity of a female mosquito. Affecting the fertility or fecundity of a mosquito typically does not kill the mosquito but affects the amount or quality of eggs the mosquito lays, as well as the ability to produce viable and/or fertile progeny. Thus, fertility refers to the ability of a population of female mosquitoes to yield eggs. Fecundity refers to a reduction in the number of progeny produced from the eggs.

Thus, fertility refers to the “ability” of a male and a female to reproduce a viable offspring.

The female mosquito may lay a reduced amount of eggs as compared to a female mosquito not affected by the larvicide composition of the invention. Alternatively, the quality of the eggs laid by the female mosquito may be damaged, e.g. the eggs may not hatch or may hatch at a reduced amount (e.g. 10%, 20%, 30%, 40%, 50%, 60%, 70%, 80%, 90% or 100% reduction in hatching as compared to eggs of a female mosquito not affected by the larvicide composition of the invention).

A population of female mosquitoes receiving the larvicide composition of the invention is considered to have sufficiently decreased fertility or fecundity if at least 30%, 40%, 50%, 60%, 70%, 80%, 90% or 100% of the females in the population are infertile, e.g., unable to produce viable eggs.

Thus, the larvicide of the invention may generate a biased population of adult mosquitoes.

In addition the term may refer to rendering a mosquito at any stage, including adulthood, more susceptible to a pesticide as compared to the susceptibility of a mosquito of the same species and developmental stage which hasn't been treated with the nucleic acid larvicide.

As used herein, the term “larvicidally effective” is used to indicate an amount or concentration of the nucleic acid larvicide which is sufficient to reduce the number of mosquitoes in a geographic locus as compared to a corresponding geographic locus in the absence of the amount or concentration of the composition.

Thus the nucleic acid larvicide of some embodiments of the invention down-regulates a target gene selected from the group consisting of:

(i) affecting larval survival;

(ii) interfering with metamorphosis of larval stage to adulthood;

(iii) affecting susceptibility of mosquito larvae to a larvicide;

(iv) affecting susceptibility of an adult mosquito to an adulticide/insecticide; and

(v) affecting fertility or fecundity of a male or female mosquito.

As used herein the term “affecting” or “interfering” refers to a gene which plays a role in the above mentioned biological activity. According to a specific embodiment, the target gene is a non-redundant gene, that is, its activity is not compensated by another gene in a pathway. When needed, down-regulation of a plurality of genes (e.g., in a pathway) participating in at least one of the above-mentioned activities is contemplated (as further described hereinbelow). Alternatively, according to a specific embodiment, the plurality of target genes are from groups (i) and (ii), (i) and (iii), (i) and (iv), (i) and (v), (ii) and (iii), (ii) and (iv), (ii) and (v), (iii) and (v) and (iv) and (v) and more.

The target gene may comprise a nucleic acid sequence which is transcribed to an mRNA which codes for a polypeptide.

Alternatively, the target gene can be a non-coding gene such as a miRNA or a siRNA.

According to a specific embodiment, the target gene is endogenous to the larvae.

According to a specific embodiment, the target gene is endogenous to the pathogen.

As used herein “endogenous” refers to a gene which expression (mRNA or protein) takes place in the larvae or the pathogen. Typically, the endogenous gene is naturally expressed in the larvae or the pathogen.

Below provided are exemplary genes. Orthologs and homologs are also contemplated according to the present teachings.

Homologous sequences include both orthologous and paralogous sequences. The term “paralogous” relates to gene-duplications within the genome of a species leading to paralogous genes. The term “orthologous” relates to homologous genes in different organisms due to ancestral relationship. Thus, orthologs are evolutionary counterparts derived from a single ancestral gene in the last common ancestor of given two species (Koonin E V and Galperin M Y (Sequence—Evolution—Function: Computational Approaches in Comparative Genomics. Boston: Kluwer Academic; 2003. Chapter 2, Evolutionary Concept in Genetics and Genomics. Available from: ncbi (dot) nlm (dot) nih (dot) gov/books/NBK20255) and therefore have great likelihood of having the same function.

The term “ortholog” (also called orthologous genes) refers to genes in different species derived from a common ancestry (due to speciation).

According to a specific embodiment, the homolog sequences are at least 60%, 65%, 70%, 75%, 80%, 85%, 90%, 95% or even identical to the sequences (nucleic acid or amino acid sequences) provided hereinbelow.

The nucleic acid agent will be selected according to the target larvae and hence target genes. Exemplary target genes of the invention include adulticide/larvicide targets and fertility/fecundity targets.

Exemplary target genes of the invention are listed in Tables 1-5 below.

TABLE 1

| Seq ID | Gene Symbol |
|---|---|
| 302 | AAEL001340 |
| 303 | AAEL001606 |
| 304 | AAEL002425 |
| 305 | AAEL002792 |
| 306 | AAEL003660 |
| 307 | AAEL004696 |
| 308 | AAEL004974 |
| 309 | AAEL006254 |
| 310 | AAEL006488 |
| 311 | AAEL006492 |
| 312 | AAEL008042 |
| 313 | AAEL008587 |
| 314 | AAEL008844 |
| 315 | AAEL008924 |
| 316 | AAEL008958 |
| 317 | AAEL009114 |
| 318 | AAEL009174 |
| 319 | AAEL009340 |
| 320 | AAEL009969 |
| 321 | AAEL010565 |
| 322 | AAEL010789 |
| 323 | AAEL010792 |
| 324 | AAEL011474 |
| 325 | AAEL011478 |
| 326 | AAEL011663 |
| 327 | AAEL011757 |
| 328 | AAEL011921 |
| 329 | AAEL014330 |
| 330 | AGAP000460 |
| 331 | AGAP000460 |
| 332 | AGAP000460 |
| 333 | AGAP000471 |
| 334 | AGAP000471 |
| 335 | AGAP000471 |
| 336 | AGAP000662 |
| 337 | AGAP000662 |
| 338 | AGAP000662 |
| 339 | AGAP001177 |
| 340 | AGAP001177 |
| 341 | AGAP001177 |
| 342 | AGAP001179 |
| 343 | AGAP001179 |
| 344 | AGAP001179 |
| 345 | AGAP001271 |
| 346 | AGAP001271 |
| 347 | AGAP001271 |
| 348 | AGAP001278 |
| 349 | AGAP001278 |
| 350 | AGAP001278 |
| 351 | AGAP001293 |
| 352 | AGAP001293 |
| 353 | AGAP001293 |
| 354 | AGAP001335 |
| 355 | AGAP001335 |
| 356 | AGAP001335 |
| 357 | AGAP001337 |
| 358 | AGAP001337 |
| 359 | AGAP001337 |
| 360 | AGAP001339 |
| 361 | AGAP001339 |
| 362 | AGAP001339 |
| 363 | AGAP001367 |
| 364 | AGAP001367 |
| 365 | AGAP001367 |
| 366 | AGAP001388 |
| 367 | AGAP001388 |
| 368 | AGAP001388 |
| 369 | AGAP001463 |
| 370 | AGAP001463 |
| 371 | AGAP001463 |
| 372 | AGAP001478 |
| 373 | AGAP001478 |
| 374 | AGAP001478 |
| 375 | AGAP001481 |
| 376 | AGAP001481 |
| 377 | AGAP001481 |
| 378 | AGAP001498 |
| 379 | AGAP001498 |
| 380 | AGAP001498 |
| 381 | AGAP002471 |
| 382 | AGAP002471 |
| 383 | AGAP002471 |
| 384 | AGAP002801 |
| 385 | AGAP004050 |
| 386 | AGAP004416 |
| 387 | AGAP004416 |
| 388 | AGAP004416 |
| 389 | AGAP004645 |
| 390 | AGAP004930 |
| 391 | AGAP006887 |
| 392 | AGAP006887 |
| 393 | AGAP006887 |
| 394 | AGAP007963 |
| 395 | AGAP008806 |
| 396 | CPIJ001185 |
| 397 | CPIJ001186 |
| 398 | CPIJ001187 |
| 399 | CPIJ001560 |
| 400 | CPIJ003158 |
| 401 | CPIJ003766 |
| 402 | CPIJ004057 |
| 403 | CPIJ004058 |
| 404 | CPIJ004318 |
| 405 | CPIJ005975 |
| 406 | CPIJ005976 |
| 407 | CPIJ007071 |
| 408 | CPIJ007072 |
| 409 | CPIJ007101 |
| 410 | CPIJ007172 |
| 411 | CPIJ007789 |
| 412 | CPIJ008481 |
| 413 | CPIJ008673 |
| 414 | CPIJ009011 |
| 415 | CPIJ009270 |
| 416 | CPIJ011557 |
| 417 | CPIJ011558 |
| 418 | CPIJ011708 |
| 419 | CPIJ012810 |
| 420 | CPIJ013126 |
| 421 | CPIJ015620 |
| 422 | CPIJ015622 |
| 423 | CPIJ017065 |
| 424 | CPIJ017887 |
| 425 | CPIJ019248 |
| 426 | CPIJ019249 |
| 427 | FBgn0127180 |

TABLE 2A

| | Seq | | |
|---|---|---|---|
| | ID | Gene Symbol | Annotation |
| Enzymes | 55 | AAEL012664 | prolylcarboxypeptidase, putative |
| | 56 | AAEL002909 | lysosomal acid lipase, putative |
| | 57 | AAEL005127 | ribonuclease UK114, putative |
| | 58 | AAEL012636 | cytochrome b5, putative |
| | 59 | AAEL010276 | aminomethyltransferase |
| | 60 | AAEL013640 | lung carbonyl reductase |
| | 61 | AAEL005416 | oxidase/peroxidase |
| | 62 | AAEL013499 | prophenoloxidase |
| | 63 | AAEL003716 | ribonuclease UK114, putative |
| | 64 | AAEL012579 | aspartate aminotransferase |
| | 65 | AAEL002600 | serine protease |
| | 66 | AAEL005610 | mitochondrial ATP synthase b |
| | | | chain |
| | 67 | AAEL006446 | trehalose-6-phosphate synthase |
| | 68 | AAEL008770 | proteasome subunit beta type |
| | 69 | AAEL001427 | short-chain dehydrogenase |
| | 70 | AAEL013279 | peptidyl-prolyl cis-trans isomerase |
| | | | (cyclophilin) |
| | 71 | AAEL009875 | alanine aminotransferase |
| | 72 | AAEL005793 | AMP dependent ligase |
| | 73 | AAEL007868 | ubiquinol-cytochrome c reductase |
| | | | complex 14 kd protein |
| | 74 | AAEL008072 | NADH-plastoquinone |
| | | | oxidoreductase |
| | 75 | AAEL009324 | hydroxyacyl dehydrogenase |
| | 76 | AAEL008217 | serine-type enodpeptidase, |
| | 77 | AAEL014944 | cytochrome c oxidase polypeptide |
| | 78 | AAEL010819 | vacuolar ATP synthase subunit H |
| | 79 | AAEL010500 | glutathione-s-transferase theta, gst |
| Transport | 80 | AAEL005929 | ATP-binding cassette transporter |
| | 81 | AAEL008381 | oligopeptide transporter |
| | 82 | AAEL001626 | zinc/iron transporter |
| | 83 | AAEL012702 | ATP-binding cassette sub-family |
| | | | A member 3, putative |
| others | 84 | AAEL015515 | antibacterial peptide, putative |
| | 85 | AAEL002295 | leucine-rich transmembrane |
| | | | protein |
| | 86 | AAEL009556 | Niemann-Pick Type C-2, putative |
| | 87 | AAEL005159 | latent nuclear antigen, putative |
| | 88 | AAEL007325 | Mob3B protein, putative |
| | 89 | AAEL000679 | NEDD8, putative |
| | 90 | AAEL009209 | galactose-specific C-type lectin, |
| | | | putative |
| | 91 | AAEL001826 | odorant-binding protein 56a, |
| | | | putative |
| | 92 | AAEL002961 | Osiris, putative |
| | 93 | AAEL006830 | yellow protein precursor |
| | 94 | AAEL005772 | odorant-binding protein 99c, |
| | | | putative |
| | 95 | AAEL002813 | coupling factor, putative |
| | 96 | AAEL011090 | complement component |
| | 97 | AAEL012230 | flagellar protein, putative |
| Hypothetical | 98 | AAEL011252 | conserved hypothetical protein |
| proteins | 99 | AAEL014506 | conserved hypothetical protein |
| | 100 | AAEL003216 | conserved hypothetical protein |
| | 101 | AAEL003241 | conserved hypothetical protein |
| | 102 | AAEL007507 | conserved hypothetical protein |
| | 103 | AAEL003064 | conserved hypothetical protein |
| | 104 | AAEL010678 | conserved hypothetical protein |
| | 105 | AAEL000269 | conserved hypothetical protein |
| | 106 | AAEL006053 | conserved hypothetical protein |
| | 107 | AAEL008750 | conserved hypothetical protein |
| | 108 | AAEL010128 | conserved hypothetical protein |
| | 109 | AAEL002898 | conserved hypothetical protein |
| | 110 | AAEL007631 | conserved hypothetical protein |
| | 111 | AAEL003479 | conserved hypothetical protein |
| | 112 | AAEL013777 | conserved hypothetical protein |
| | 113 | AAEL003428 | conserved hypothetical protein |
| | 114 | AAEL014529 | conserved hypothetical protein |
| | 115 | AAEL012645 | conserved hypothetical protein |
| | 116 | AAEL004809 | conserved hypothetical protein |
| | 117 | AAEL004343 | conserved hypothetical protein |
| | 118 | AAEL003160 | conserved hypothetical protein |
| | 119 | AAEL012357 | conserved hypothetical protein |
| | 120 | AAEL009009 | conserved hypothetical protein |
| | 121 | AAEL013793 | conserved hypothetical protein |
| | 122 | AAEL002623 | conserved hypothetical protein |
| | 123 | AAEL010163 | conserved hypothetical protein |
| | 124 | AAEL002449 | conserved hypothetical protein |
| | 125 | AAEL002302 | conserved hypothetical protein |
| | 126 | AAEL008039 | conserved hypothetical protein |
| | 127 | AAEL008073 | conserved hypothetical protein |
| | 128 | AAEL007444 | conserved hypothetical protein |
| | 129 | AAEL005171 | conserved hypothetical protein |
| | 130 | AAEL006771 | conserved hypothetical protein |
| | 131 | AAEL015140 | conserved hypothetical protein |
| | 132 | AAEL001851 | conserved hypothetical protein |
| | 133 | AAEL005558 | conserved hypothetical protein |
| | 134 | AAEL002933 | conserved hypothetical protein |
| | 135 | AAEL003225 | conserved hypothetical protein |
| | 136 | AAEL001692 | conserved hypothetical protein |
| | 137 | AAEL007592 | conserved hypothetical protein |
| | 138 | AAEL005457 | conserved hypothetical protein |
| | 139 | AAEL006494 | conserved hypothetical protein |
| | 140 | AAEL013780 | conserved hypothetical protein |
| | 141 | AAEL009257 | conserved hypothetical protein |
| | 142 | AAEL000445 | conserved hypothetical protein |
| | 143 | AAEL002955 | conserved hypothetical protein |
| | 144 | AAEL002875 | conserved hypothetical protein |
| | 145 | AAEL000304 | conserved hypothetical protein |
| | 146 | AAEL000792 | conserved hypothetical protein |
| | 147 | AAEL003936 | conserved hypothetical protein |
| | 148 | AAEL006686 | conserved hypothetical protein |
| | 149 | AAEL001677 | conserved hypothetical protein |
| | 150 | AAEL000419 | conserved hypothetical protein |
| | 151 | AAEL007648 | conserved hypothetical protein |
| | 152 | AAEL006270 | conserved hypothetical protein |
| | 153 | AAEL013377 | conserved hypothetical protein |
| | 154 | AAEL002619 | conserved hypothetical protein |
| | 155 | AAEL012866 | conserved hypothetical protein |
| | 156 | AAEL014445 | conserved hypothetical protein |
| | 157 | AAEL001065 | conserved hypothetical protein |
| | 158 | AAEL011333 | conserved hypothetical protein |
| | 159 | AAEL011078 | conserved hypothetical protein |
| | 160 | AAEL010315 | conserved hypothetical protein |
| | 161 | AAEL005270 | conserved hypothetical protein |
| | 162 | AAEL004449 | conserved hypothetical protein |
| | 163 | AAEL000896 | conserved hypothetical protein |
| | 164 | AAEL010724 | conserved hypothetical protein |
| | 165 | AAEL008802 | conserved hypothetical protein |

TABLE 2B

| | Gene symbol | Gene Name |
|---|---|---|
| 430 | AAEL000043 | gustatory receptor 64e, putative |
| 431 | AAEL000020 | conserved hypothetical protein |
| 432 | AAEL000005 | hypothetical protein |
| 433 | AAEL000049 | three prime repair exonuclease 1, putative |
| 434 | AAEL000053 | myotubularin |
| 435 | AAEL000033 | conserved hypothetical protein |
| 436 | AAEL000046 | conserved hypothetical protein |
| 437 | AAEL000054 | mixed-lineage leukemia protein, mll |
| 438 | AAEL000011 | conserved hypothetical protein |
| 439 | AAEL000055 | conserved hypothetical protein |
| 440 | AAEL000543 | C-Type Lectin (CTL) - mannose binding. |
| 441 | AAEL000559 | glycosyl transferase |
| 442 | AAEL000562 | conserved hypothetical protein |
| 443 | AAEL000554 | fasciclin, putative |
| 444 | AAEL014092 | conserved hypothetical protein |
| 445 | AAEL014105 | hypothetical protein |
| 446 | AAEL014110 | sulfite reductase |
| 447 | AAEL003935 | conserved hypothetical protein |
| 448 | AAEL003940 | hypothetical protein |
| 449 | AAEL014125 | nk homeobox protein |
| 450 | AAEL014128 | hypothetical protein |
| 451 | AAEL003971 | conserved hypothetical protein |
| 452 | AAEL003959 | short-chain dehydrogenase |
| 453 | AAEL014161 | amino acids transporter |
| 454 | AAEL014163 | conserved hypothetical protein |
| 455 | AAEL014183 | hypothetical protein |
| 456 | AAEL003983 | adenylate cyclase |
| 457 | AAEL003979 | deadenylation factor EDEN-BP, putative |
| 458 | AAEL014196 | conserved hypothetical protein |
| 459 | AAEL003988 | hypothetical protein |
| 460 | AAEL003986 | conserved hypothetical protein |
| 461 | AAEL014222 | low-density lipoprotein receptor (ldl) |
| 462 | AAEL014229 | conserved hypothetical protein |
| 463 | AAEL014237 | conserved hypothetical protein |
| 464 | AAEL014250 | insect replication protein a |
| 465 | AAEL004030 | conserved hypothetical protein |
| 466 | AAEL004018 | conserved hypothetical protein |
| 467 | AAEL014257 | hypothetical protein |
| 468 | AAEL014268 | hypothetical protein |
| 469 | AAEL014271 | conserved hypothetical protein |
| 470 | AAEL014272 | molybdopterin cofactor sulfurase (mosc) |
| 471 | AAEL014276 | conserved hypothetical protein |
| 472 | AAEL014283 | conserved hypothetical protein |
| 473 | AAEL014291 | hypothetical protein |
| 474 | AAEL004055 | conserved hypothetical protein |
| 475 | AAEL004076 | Ubiquitin-like modifier-activating enzyme 5 |
| | | (Ubiquitin-activating enzyme 5) |
| 476 | AAEL004068 | hypothetical protein |
| 477 | AAEL014302 | conserved hypothetical protein |
| 478 | AAEL014306 | hypothetical protein |
| 479 | AAEL014304 | hypothetical protein |
| 480 | AAEL014318 | conserved hypothetical protein |
| 481 | AAEL014314 | DNA primase |
| 482 | AAEL014317 | conserved hypothetical protein |
| 483 | AAEL014316 | conserved hypothetical protein |
| 484 | AAEL014319 | hypothetical protein |
| 485 | AAEL004084 | conserved hypothetical protein |
| 486 | AAEL004094 | pou domain |
| 487 | AAEL004104 | hypothetical protein |
| 488 | AAEL014324 | conserved hypothetical protein |
| 489 | AAEL004130 | conserved hypothetical protein |
| 490 | AAEL004129 | conserved hypothetical protein |
| 491 | AAEL014341 | metallocarboxypeptidase, putative |
| 492 | AAEL014342 | hypothetical protein |
| 493 | AAEL004190 | hypothetical protein |
| 494 | AAEL004178 | ribose-phosphate pyrophosphokinase 1, |
| 495 | AAEL004172 | tubulin alpha chain |
| 496 | AAEL004176 | microtubule binding protein, putative |
| 497 | AAEL004150 | fibrinogen and fibronectin |
| 498 | AAEL004188 | conserved hypothetical protein |
| 499 | AAEL004191 | selenocysteine-specific elongation factor |
| 500 | AAEL014367 | hypothetical protein |
| 501 | AAEL014373 | GPCR Dopamine Family |
| 502 | AAEL014389 | conserved hypothetical protein |
| 503 | AAEL000584 | sex-determining region y protein, sry |
| 504 | AAEL000589 | serine/threonine protein kinase |
| 505 | AAEL000585 | hypothetical protein |
| 506 | AAEL017305 | odorant receptor |
| 507 | AAEL014402 | conserved hypothetical protein |
| 508 | AAEL014411 | cytochrome P450 |
| 509 | AAEL004234 | conserved hypothetical protein |
| 510 | AAEL014428 | ABC transporter |
| 511 | AAEL014431 | conserved hypothetical protein |
| 512 | AAEL014441 | conserved hypothetical protein |
| 513 | AAEL004253 | hypothetical protein |
| 514 | AAEL004300 | conserved hypothetical protein |
| 515 | AAEL004302 | conserved hypothetical protein |
| 516 | AAEL004296 | conserved hypothetical protein |
| 517 | AAEL004317 | hypothetical protein |
| 518 | AAEL004315 | hypothetical protein |
| 519 | AAEL004348 | NF-180, putative |
| 520 | AAEL004328 | origin recognition complex subunit |
| 521 | AAEL014524 | DNA replication licensing factor MCM4 |
| 522 | AAEL014533 | conserved hypothetical protein |
| 523 | AAEL014536 | embryonic ectoderm development protein |
| 524 | AAEL014548 | Thioredoxin Peroxidase. |
| 525 | AAEL014557 | homeobox protein cdx |
| 526 | AAEL004386 | heme peroxidase |
| 527 | AAEL004390 | heme peroxidase |
| 528 | AAEL004399 | GPCR Glycoprotein Hormone Family |
| 529 | AAEL017431 | hypothetical protein |
| 530 | AAEL004388 | heme peroxidase |
| 531 | AAEL004396 | GPCR Octopamine/Tyramine Family |
| 532 | AAEL014572 | tetraspanin 97e |
| 533 | AAEL017559 | hypothetical protein |
| 534 | AAEL014577 | conserved hypothetical protein |
| 535 | AAEL014589 | reticulocalbin |
| 536 | AAEL004416 | histone deacetylase |
| 537 | AAEL004443 | nitrilase, putative |
| 538 | AAEL004437 | dual-specificity protein phosphatase, putative |
| 539 | AAEL004429 | conserved hypothetical protein |
| 540 | AAEL014627 | short-chain dehydrogenase |
| 541 | AAEL014633 | conserved hypothetical protein |
| 542 | AAEL000604 | hypothetical protein |
| 543 | AAEL000601 | hypothetical protein |
| 544 | AAEL004482 | conserved hypothetical protein |
| 545 | AAEL004462 | hypothetical protein |
| 546 | AAEL004468 | hypothetical protein |
| 547 | AAEL004479 | organic cation transporter |
| 548 | AAEL004486 | valacyclovir hydrolase |
| 549 | AAEL004510 | hypothetical protein |
| 550 | AAEL004493 | ribosome biogenesis protein tsr1 (20S rRNA |
| | | accumulation protein 1) |
| 551 | AAEL017243 | hypothetical protein |
| 552 | AAEL004518 | Clip-Domain Serine Protease family C |
| 553 | AAEL004537 | hypothetical protein |
| 554 | AAEL004539 | hypothetical protein |
| 555 | AAEL014693 | conserved hypothetical protein |
| 556 | AAEL004559 | synaptosomal associated protein |
| 557 | AAEL004562 | DNA polymerase eta |
| 558 | AAEL004592 | tyrosine-protein kinase src64b |
| 559 | AAEL004598 | hypothetical protein |
| 560 | AAEL014744 | conserved hypothetical protein |
| 561 | AAEL004627 | hypothetical protein |
| 562 | AAEL004635 | hypothetical protein |
| 563 | AAEL004638 | conserved hypothetical protein |
| 564 | AAEL004645 | hypothetical protein |
| 565 | AAEL004650 | hypothetical protein |
| 566 | AAEL017478 | hypothetical protein |
| 567 | AAEL004623 | band 4.1-like protein 5, putative |
| 568 | AAEL004642 | hypothetical protein |
| 569 | AAEL014756 | hypothetical protein |
| 570 | AAEL004680 | nuclear lamin L1 alpha, putative |
| 571 | AAEL004698 | DNA primase large subunit |
| 572 | AAEL004697 | synoviolin |
| 573 | AAEL014791 | hypothetical protein |
| 574 | AAEL014800 | conserved hypothetical protein |
| 575 | AAEL014814 | conserved hypothetical protein |
| 576 | AAEL017122 | hypothetical protein |
| 577 | AAEL017102 | hypothetical protein |
| 578 | AAEL014823 | conserved hypothetical protein |
| 579 | AAEL014825 | conserved hypothetical protein |
| 580 | AAEL014835 | conserved hypothetical protein |
| 581 | AAEL004777 | Glycoprotein Hormone Family |
| 582 | AAEL017453 | hypothetical protein |
| 583 | AAEL004748 | pupal cuticle protein, putative |
| 584 | AAEL004771 | pupal cuticle protein, putative |
| 585 | AAEL004782 | pupal cuticle protein, putative |
| 586 | AAEL014844 | conserved hypothetical protein |
| 587 | AAEL000666 | pmp22 peroxisomal membrane protein, putative |
| 588 | AAEL000672 | cyclin a |
| 589 | AAEL000664 | hypothetical protein |
| 590 | AAEL000662 | conserved hypothetical protein |
| 591 | AAEL000649 | conserved hypothetical protein |
| 592 | AAEL000658 | conserved hypothetical protein |
| 593 | AAEL000675 | hypothetical protein |
| 594 | AAEL004796 | hypothetical protein |
| 595 | AAEL004789 | hypothetical protein |
| 596 | AAEL004818 | conserved hypothetical protein |
| 597 | AAEL004805 | potassium-dependent sodium-calcium exchanger, |
| | | putative |
| 598 | AAEL004821 | potassium-dependent sodium-calcium exchanger, |
| | | putative |
| 599 | AAEL004842 | conserved hypothetical protein |
| 600 | AAEL004837 | hypothetical protein |
| 601 | AAEL004850 | hypothetical protein |
| 602 | AAEL004835 | conserved hypothetical protein |
| 603 | AAEL004858 | conserved hypothetical protein |
| 604 | AAEL004841 | conserved hypothetical protein |
| 605 | AAEL004882 | conserved hypothetical protein |
| 606 | AAEL004892 | conserved hypothetical protein |
| 607 | AAEL004864 | hypothetical protein |
| 608 | AAEL014904 | DEAD box ATP-dependent RNA helicase |
| 609 | AAEL014908 | conserved hypothetical protein |
| 610 | AAEL014911 | synaptic vesicle protein |
| 611 | AAEL017811 | RNase MRP |
| 612 | AAEL014918 | lysosomal acid lipase, putative |
| 613 | AAEL014924 | cytochrome P450 |
| 614 | AAEL014925 | conserved hypothetical protein |
| 615 | AAEL004914 | conserved hypothetical protein |
| 616 | AAEL004903 | conserved hypothetical protein |
| 617 | AAEL014927 | sodium/chloride dependent transporter |
| 618 | AAEL016998 | hypothetical protein |
| 619 | AAEL014946 | protease U48 caax prenyl protease rce1 |
| 620 | AAEL014956 | internalin A, putative |
| 621 | AAEL004955 | hypothetical protein |
| 622 | AAEL004949 | elongase, putative |
| 623 | AAEL004976 | conserved hypothetical protein |
| 624 | AAEL004970 | conserved hypothetical protein |
| 625 | AAEL005006 | cytochrome P450 |
| 626 | AAEL005000 | conserved hypothetical protein |
| 627 | AAEL005007 | hypothetical protein |
| 628 | AAEL005009 | groucho protein (enhancer of split) |
| 629 | AAEL014984 | adult cuticle protein, putative |
| 630 | AAEL014986 | conserved hypothetical protein |
| 631 | AAEL014992 | rab gdp/GTP exchange factor |
| 632 | AAEL014989 | peptidoglycan recognition protein-1, putative |
| 633 | AAEL005040 | conserved hypothetical protein |
| 634 | AAEL005033 | conserved hypothetical protein |
| 635 | AAEL015011 | hypothetical protein |
| 636 | AAEL000692 | partner of sld5 |
| 637 | AAEL005078 | zinc finger protein |
| 638 | AAEL005049 | heterogeneous nuclear ribonucleoprotein |
| 639 | AAEL005070 | conserved hypothetical protein |
| 640 | AAEL005087 | hypothetical protein |
| 641 | AAEL015018 | toll |
| 642 | AAEL015035 | transcription enhancer factor, putative |
| 643 | AAEL015038 | palmitoyl-protein thioesterase |
| 644 | AAEL015047 | hypothetical protein |
| 645 | AAEL005123 | hypothetical protein |
| 646 | AAEL005110 | conserved hypothetical protein |
| 647 | AAEL005115 | hypothetical protein |
| 648 | AAEL005128 | hypothetical protein |
| 649 | AAEL005103 | conserved hypothetical protein |
| 650 | AAEL005145 | conserved hypothetical protein |
| 651 | AAEL015071 | gustatory receptor 64a, putative |
| 652 | AAEL005212 | hypothetical protein |
| 653 | AAEL005197 | conserved hypothetical protein |
| 654 | AAEL005175 | lipin |
| 655 | AAEL005217 | membrin |
| 656 | AAEL005215 | conserved hypothetical protein |
| 657 | AAEL005235 | conserved hypothetical protein |
| 658 | AAEL005238 | mck1 |
| 659 | AAEL015083 | conserved hypothetical protein |
| 660 | AAEL015080 | conserved hypothetical protein |
| 661 | AAEL005241 | lateral signaling target protein 2 |
| 662 | AAEL005259 | conserved hypothetical protein |
| 663 | AAEL005261 | conserved hypothetical protein |
| 664 | AAEL015107 | conserved hypothetical protein |
| 665 | AAEL005286 | hypothetical protein |
| 666 | AAEL015119 | cuticle protein, putative |
| 667 | AAEL000749 | conserved hypothetical protein |
| 668 | AAEL005326 | conserved hypothetical protein |
| 669 | AAEL005312 | conserved hypothetical protein |
| 670 | AAEL015136 | Niemann-Pick Type C-2, putative |
| 671 | AAEL005348 | hypothetical protein |
| 672 | AAEL005351 | leucine-rich transmembrane protein |
| 673 | AAEL005362 | hypothetical protein |
| 674 | AAEL005364 | adaptin, alpha/gamma/epsilon |
| 675 | AAEL015161 | conserved hypothetical protein |
| 676 | AAEL005383 | Adenosine monophosphate-protein transferase |
| | | FICD homolog (EC 2.7.7.n1) |
| 677 | AAEL005420 | p15-2a protein, putative |
| 678 | AAEL017015 | hypothetical protein |
| 679 | AAEL005430 | hypothetical protein |
| 680 | AAEL005439 | mical |
| 681 | AAEL005452 | conserved hypothetical protein |
| 682 | AAEL005474 | hypothetical protein |
| 683 | AAEL000773 | kinesin heavy chain |
| 684 | AAEL000754 | conserved hypothetical protein |
| 685 | AAEL000779 | hypothetical protein |
| 686 | AAEL000776 | conserved hypothetical protein |
| 687 | AAEL000790 | conserved hypothetical protein |
| 688 | AAEL000793 | venom allergen |
| 689 | AAEL000769 | arginine/serine-rich splicing factor |
| 690 | AAEL005513 | mothers against dpp protein |
| 691 | AAEL015222 | adult cuticle protein, putative |
| 692 | AAEL015232 | GTP-binding protein rit |
| 693 | AAEL005549 | hypothetical protein |
| 694 | AAEL005588 | conserved hypothetical protein |
| 695 | AAEL005584 | hypothetical protein |
| 696 | AAEL015243 | hypothetical protein |
| 697 | AAEL005638 | conserved hypothetical protein |
| 698 | AAEL005637 | vegetatible incompatibility protein HET-E-1, |
| | | putative |
| 699 | AAEL017446 | gustatory receptor Gr33a |
| 700 | AAEL005656 | myosin heavy chain, nonmuscle or smooth |
| | | muscle |
| 701 | AAEL015270 | hypothetical protein |
| 702 | AAEL005668 | conserved hypothetical protein |
| 703 | AAEL000816 | carbonic anhydrase |
| 704 | AAEL000805 | conserved hypothetical protein |
| 705 | AAEL000819 | hypothetical protein |
| 706 | AAEL000800 | microsomal dipeptidase |
| 707 | AAEL005707 | gonadotropin inducible transcription factor |
| 708 | AAEL005684 | chitinase |
| 709 | AAEL005724 | conserved hypothetical protein |
| 710 | AAEL005716 | conserved hypothetical protein |
| 711 | AAEL005721 | conserved hypothetical protein |
| 712 | AAEL015293 | zinc finger protein |
| 713 | AAEL005786 | conserved hypothetical protein |
| 714 | AAEL005775 | cytochrome P450 |
| 715 | AAEL005796 | eukaryotic translation initiation factor 4e type |
| 716 | AAEL005804 | hypothetical protein |
| 717 | AAEL005805 | alanyl aminopeptidase |
| 718 | AAEL005808 | alanyl aminopeptidase |
| 719 | AAEL005853 | amino acid transporter |
| 720 | AAEL005856 | signal recognition particle receptor alpha subunit |
| | | (sr-alpha) |
| 721 | AAEL005859 | amino acid transporter |
| 722 | AAEL000903 | Enhancer of yellow 2 transcription factor |
| 723 | AAEL000912 | conserved hypothetical protein |
| 724 | AAEL000889 | hypothetical protein |
| 725 | AAEL000884 | eukaryotic translation initiation factor 2 alpha |
| | | kinase 1 (heme-regulated eukaryotic initiation |
| | | factor eif-2-alpha kinase) |
| 726 | AAEL000905 | hypothetical protein |
| 727 | AAEL000923 | conserved hypothetical protein |
| 728 | AAEL005938 | hypothetical protein |
| 729 | AAEL005936 | conserved hypothetical protein |
| 730 | AAEL005932 | conserved hypothetical protein |
| 731 | AAEL005924 | hypothetical protein |
| 732 | AAEL017009 | odorant receptor |
| 733 | AAEL005922 | hypothetical protein |
| 734 | AAEL005916 | hypothetical protein |
| 735 | AAEL015327 | conserved hypothetical protein |
| 736 | AAEL015330 | hypothetical protein |
| 737 | AAEL015336 | conserved hypothetical protein |
| 738 | AAEL005990 | adrenodoxin reductase, putative |
| 739 | AAEL006010 | conserved hypothetical protein |
| 740 | AAEL005998 | rap GTPase-activating protein |
| 741 | AAEL006031 | conserved hypothetical protein |
| 742 | AAEL006023 | Vanin-like protein 1 precursor, putative |
| 743 | AAEL006030 | hypothetical protein |
| 744 | AAEL006037 | hypothetical protein |
| 745 | AAEL006045 | reticulon/nogo receptor |
| 746 | AAEL006055 | potassium channel interacting protein |
| 747 | AAEL006084 | conserved hypothetical protein |
| 748 | AAEL006091 | rab6 |
| 749 | AAEL006098 | conserved hypothetical protein |
| 750 | AAEL000938 | conserved hypothetical protein |
| 751 | AAEL000945 | conserved hypothetical protein |
| 752 | AAEL000971 | smile protein |
| 753 | AAEL000973 | conserved hypothetical protein |
| 754 | AAEL000932 | conserved hypothetical protein |
| 755 | AAEL000953 | conserved hypothetical protein |
| 756 | AAEL000964 | regulatory factor X-associated ankyrin-containing |
| | | protein, putative |
| 757 | AAEL000934 | clathrin light chain |
| 758 | AAEL006111 | hypothetical protein |
| 759 | AAEL006140 | mitosis inhibitor protein kinase |
| 760 | AAEL006138 | hypothetical protein |
| 761 | AAEL006130 | hypothetical protein |
| 762 | AAEL006208 | conserved hypothetical protein |
| 763 | AAEL006211 | conserved hypothetical protein |
| 764 | AAEL006219 | heparan sulphate 2-o-sulfotransferase |
| 765 | AAEL006239 | glycerol kinase |
| 766 | AAEL006243 | hypothetical protein |
| 767 | AAEL006241 | sugar transporter |
| 768 | AAEL015376 | conserved hypothetical protein |
| 769 | AAEL006258 | pickpocket |
| 770 | AAEL015380 | conserved hypothetical protein |
| 771 | AAEL006277 | conserved hypothetical protein |
| 772 | AAEL006279 | hypothetical protein |
| 773 | AAEL006262 | mitochondrial carrier protein |
| 774 | AAEL006298 | conserved hypothetical protein |
| 775 | AAEL006303 | integral membrane protein, Tmp21-I (p23), |
| | | putative |
| 776 | AAEL006286 | conserved hypothetical protein |
| 777 | AAEL000113 | conserved hypothetical protein |
| 778 | AAEL000128 | P130 |
| 779 | AAEL000141 | immunophilin FKBP46, putative |
| 780 | AAEL000154 | conserved hypothetical protein |
| 781 | AAEL000999 | DNA replication licensing factor MCM7 |
| 782 | AAEL017422 | hypothetical protein |
| 783 | AAEL000974 | zinc finger protein |
| 784 | AAEL000980 | hypothetical protein |
| 785 | AAEL006308 | px serine/threonine kinase (pxk) |
| 786 | AAEL006321 | 1-acylglycerol-3-phosphate acyltransferase |
| 787 | AAEL006309 | conserved hypothetical protein |
| 788 | AAEL006341 | conserved hypothetical protein |
| 789 | AAEL006326 | deoxyribonuclease I, putative |
| 790 | AAEL006340 | conserved hypothetical protein |
| 791 | AAEL006360 | conserved hypothetical protein |
| 792 | AAEL006370 | amsh |
| 793 | AAEL006371 | oviductin |
| 794 | AAEL006392 | hypothetical protein |
| 795 | AAEL006405 | hypothetical protein |
| 796 | AAEL006450 | integral membrane protein, putative |
| 797 | AAEL006447 | GATA transcription factor (GATAb) |
| 798 | AAEL006460 | par-6 gamma |
| 799 | AAEL006455 | calcium-activated potassium channel |
| 800 | AAEL006498 | long wavelength sensitive opsin |
| 801 | AAEL006523 | crk |
| 802 | AAEL006538 | peroxisomal membrane protein 2, pxmp2 |
| 803 | AAEL006556 | hypothetical protein |
| 804 | AAEL006564 | mitochondrial RNA splicing protein |
| 805 | AAEL006581 | juvenile hormone-inducible protein, putative |
| 806 | AAEL015414 | hypothetical protein |
| 807 | AAEL006603 | conserved hypothetical protein |
| 808 | AAEL006660 | conserved hypothetical protein |
| 809 | AAEL006657 | hypothetical protein |
| 810 | AAEL006656 | conserved hypothetical protein |
| 811 | AAEL006654 | conserved hypothetical protein |
| 812 | AAEL006662 | hypothetical protein |
| 813 | AAEL006672 | conserved hypothetical protein |
| 814 | AAEL006684 | Putative oxidoreductase GLYR1 homolog |
| | | (EC 1.—.—.—)(Glyoxylate reductase |
| | | 1 homolog)(Nuclear protein NP60 homolog) |
| 815 | AAEL006704 | fibrinogen and fibronectin |
| 816 | AAEL006694 | hypothetical protein |
| 817 | AAEL017095 | hypothetical protein |
| 818 | AAEL006706 | conserved hypothetical protein |
| 819 | AAEL006713 | U2 snrnp auxiliary factor, small subunit |
| 820 | AAEL006727 | multisynthetase complex, auxiliary protein, |
| | | p38, putative |
| 821 | AAEL001093 | PHD finger protein |
| 822 | AAEL001053 | hypothetical protein |
| 823 | AAEL001088 | beta-1,3-galactosyltransferase |
| 824 | AAEL001067 | hypothetical protein |
| 825 | AAEL006761 | hypothetical protein |
| 826 | AAEL006766 | hypothetical protein |
| 827 | AAEL006777 | hypothetical protein |
| 828 | AAEL006759 | hypothetical protein |
| 829 | AAEL006768 | hypothetical protein |
| 830 | AAEL006801 | conserved hypothetical protein |
| 831 | AAEL017112 | hypothetical protein |
| 832 | AAEL006823 | AMP dependent ligase |
| 833 | AAEL006847 | conserved hypothetical protein |
| 834 | AAEL006876 | igf2 mRNA binding protein, putative |
| 835 | AAEL006906 | NBP2b protein, putative |
| 836 | AAEL006923 | conserved hypothetical protein |
| 837 | AAEL006931 | hypothetical protein |
| 838 | AAEL006934 | Mediator of RNA polymerase II transcription |
| | | subunit 19 (Mediator complex subunit 19) |
| 839 | AAEL006939 | smaug protein |
| 840 | AAEL001148 | homeobox protein |
| 841 | AAEL001137 | hypothetical protein |
| 842 | AAEL001121 | n-acetylgalactosaminyltransferase |
| 843 | AAEL001116 | hypothetical protein |
| 844 | AAEL001125 | p15-2b protein, putative |
| 845 | AAEL001113 | inorganic phosphate cotransporter, putative |
| 846 | AAEL006972 | hepatocellular carcinoma-associated antigen |
| 847 | AAEL006961 | lipase |
| 848 | AAEL006982 | lipase |
| 849 | AAEL006998 | conserved hypothetical protein |
| 850 | AAEL006997 | hypothetical protein |
| 851 | AAEL006986 | conserved hypothetical protein |
| 852 | AAEL007007 | DNA replication licensing factor MCM2 |
| 853 | AAEL007053 | hypothetical protein |
| 854 | AAEL007046 | mitochondrial brown fat uncoupling protein |
| 855 | AAEL007056 | btf |
| 856 | AAEL007073 | hypothetical protein |
| 857 | AAEL007075 | conserved hypothetical protein |
| 858 | AAEL007071 | conserved hypothetical protein |
| 859 | AAEL017835 | 18S_rRNA |
| 860 | AAEL007095 | adult cuticle protein, putative |
| 861 | AAEL007101 | adult cuticle protein, putative |
| 862 | AAEL007091 | single-stranded DNA-binding protein mssp-1 |
| 863 | AAEL007093 | conserved hypothetical protein |
| 864 | AAEL007097 | 4-nitrophenylphosphatase |
| 865 | AAEL007128 | sugar transporter |
| 866 | AAEL017220 | hypothetical protein |
| 867 | AAEL007134 | hypothetical protein |
| 868 | AAEL001176 | s-adenosylmethionine decarboxylase |
| 869 | AAEL017083 | hypothetical protein |
| 870 | AAEL001180 | hypothetical protein |
| 871 | AAEL001162 | conserved hypothetical protein |
| 872 | AAEL001169 | Ribosome biogenesis protein BOP1 homolog |
| 873 | AAEL017069 | hypothetical protein |
| 874 | AAEL001158 | fructose-1,6-bisphosphatase |
| 875 | AAEL001157 | light protein |
| 876 | AAEL007171 | protein phosphatase 2c |
| 877 | AAEL007152 | hypothetical protein |
| 878 | AAEL007158 | nnp-1 protein (novel nuclear protein 1) (nop52) |
| 879 | AAEL007162 | autophagy related gene |
| 880 | AAEL007173 | conserved hypothetical protein |
| 881 | AAEL007198 | Osiris, putative |
| 882 | AAEL007221 | brain-specific homeobox protein, putative |
| 883 | AAEL007229 | conserved hypothetical protein |
| 884 | AAEL007262 | hypothetical protein |
| 885 | AAEL007261 | conserved hypothetical protein |
| 886 | AAEL007270 | hypothetical protein |
| 887 | AAEL015464 | histone H1, putative |
| 888 | AAEL007287 | conserved hypothetical protein |
| 889 | AAEL007290 | conserved hypothetical protein |
| 890 | AAEL007308 | glycosyltransferase |
| 891 | AAEL007298 | conserved hypothetical protein |
| 892 | AAEL007323 | deoxyuridine 5′-triphosphate nucleotidohydrolase |
| 893 | AAEL007339 | conserved hypothetical protein |
| 894 | AAEL007333 | hypothetical protein |
| 895 | AAEL001201 | hypothetical protein |
| 896 | AAEL007399 | conserved hypothetical protein |
| 897 | AAEL007432 | serine collagenase 1 precursor, putative |
| 898 | AAEL007427 | zinc finger protein |
| 899 | AAEL007438 | dipeptidyl-peptidase |
| 900 | AAEL007448 | dipeptidyl-peptidase |
| 901 | AAEL017387 | hypothetical protein |
| 902 | AAEL007445 | conserved hypothetical protein |
| 903 | AAEL007441 | translocon-associated protein, gamma subunit |
| 904 | AAEL007447 | hypothetical protein |
| 905 | AAEL007457 | insect origin recognition complex subunit |
| 906 | AAEL007456 | zinc finger protein, putative |
| 907 | AAEL007464 | hypothetical protein |
| 908 | AAEL007470 | staufen |
| 909 | AAEL007458 | amino acid transporter |
| 910 | AAEL007476 | makorin |
| 911 | AAEL007484 | protein transport protein sec23 |
| 912 | AAEL001231 | MIND-MELD/ADAM |
| 913 | AAEL001226 | conserved hypothetical protein |
| 914 | AAEL007523 | peroxisomal n1-acetyl-spermine/spermidine |
| | | oxidase |
| 915 | AAEL007543 | hypothetical protein |
| 916 | AAEL007554 | conserved hypothetical protein |
| 917 | AAEL007563 | Dual Oxidase: Peroxidase and NADPH- |
| | | Oxidase domains. |
| 918 | AAEL007581 | Rfc5p, putative |
| 919 | AAEL007584 | conserved hypothetical protein |
| 920 | AAEL007604 | odorant-binding protein 56a, putative |
| 921 | AAEL007612 | hypothetical protein |
| 922 | AAEL007611 | hypothetical protein |
| 923 | AAEL001240 | GPCR Orphan/Putative Class B Family |
| 924 | AAEL007639 | conserved hypothetical protein |
| 925 | AAEL007657 | low-density lipoprotein receptor (ldl) |
| 926 | AAEL007656 | receptor for activated C kinase, putative |
| 927 | AAEL007658 | partitioning defective 3, par-3 |
| 928 | AAEL007674 | conserved hypothetical protein |
| 929 | AAEL007677 | phospholysine phosphohistidine inorganic |
| | | pyrophosphate phosphatase |
| 930 | AAEL007692 | conserved hypothetical protein |
| 931 | AAEL007726 | hypothetical protein |
| 932 | AAEL017004 | hypothetical protein |
| 933 | AAEL007737 | hypothetical protein |
| 934 | AAEL007757 | conserved hypothetical protein |
| 935 | AAEL007761 | chloride intracellular channel |
| 936 | AAEL007769 | hypothetical protein |
| 937 | AAEL007768 | TOLL pathway signaling. |
| 938 | AAEL007767 | Protein kintoun |
| 939 | AAEL001246 | Thymidylate kinase, putative |
| 940 | AAEL007813 | hypothetical protein |
| 941 | AAEL017497 | hypothetical protein |
| 942 | AAEL007810 | conserved hypothetical protein |
| 943 | AAEL007819 | hypothetical protein |
| 944 | AAEL017434 | hypothetical protein |
| 945 | AAEL007835 | serine/threonine protein kinase |
| 946 | AAEL007828 | palmitoyl-protein thioesterase |
| 947 | AAEL015517 | conserved hypothetical protein |
| 948 | AAEL007859 | conserved hypothetical protein |
| 949 | AAEL007862 | conserved hypothetical protein |
| 950 | AAEL007855 | hypothetical protein |
| 951 | AAEL007867 | hypothetical protein |
| 952 | AAEL007870 | hypothetical protein |
| 953 | AAEL007873 | hypothetical protein |
| 954 | AAEL007875 | hypothetical protein |
| 955 | AAEL007907 | serine/threonine protein kinase |
| 956 | AAEL007899 | spermatogenesis associated factor |
| 957 | AAEL007896 | hypothetical protein |
| 958 | AAEL007912 | conserved hypothetical protein |
| 959 | AAEL007926 | retinoid-inducible serine carboxypeptidase |
| | | (serine carboxypeptidase |
| 960 | AAEL007922 | conserved hypothetical protein |
| 961 | AAEL007921 | zinc finger protein |
| 962 | AAEL001321 | transcription factor dp |
| 963 | AAEL001296 | hypothetical protein |
| 964 | AAEL007932 | hypothetical protein |
| 965 | AAEL007939 | conserved hypothetical protein |
| 966 | AAEL007959 | conserved hypothetical protein |
| 967 | AAEL007977 | hypothetical protein |
| 968 | AAEL007991 | conserved hypothetical protein |
| 969 | AAEL007987 | SEC63 protein, putative |
| 970 | AAEL007997 | conserved hypothetical protein |
| 971 | AAEL008001 | conserved hypothetical protein |
| 972 | AAEL008020 | sorting nexin |
| 973 | AAEL017171 | hypothetical protein |
| 974 | AAEL008043 | PNR-like nuclear receptor |
| 975 | AAEL008041 | bleomycin hydrolase |
| 976 | AAEL008050 | hypothetical protein |
| 977 | AAEL008074 | hypothetical protein |
| 978 | AAEL008057 | myosin light chain kinase |
| 979 | AAEL008065 | hypothetical protein |
| 980 | AAEL000208 | copii-coated vesicle membrane protein P24 |
| 981 | AAEL000186 | conserved hypothetical protein |
| 982 | AAEL000193 | histone-lysine n-methyltransferase |
| 983 | AAEL000185 | eukaryotic translation initiation factor |
| 984 | AAEL001327 | conserved hypothetical protein |
| 985 | AAEL001348 | conserved hypothetical protein |
| 986 | AAEL001357 | hypothetical protein |
| 987 | AAEL017348 | hypothetical protein |
| 988 | AAEL008088 | conserved hypothetical protein |
| 989 | AAEL017518 | hypothetical protein |
| 990 | AAEL008101 | hypothetical protein |
| 991 | AAEL008099 | procollagen-lysine,2-oxoglutarate |
| | | 5-dioxygenase |
| 992 | AAEL008126 | GPCR Latrophilin Family |
| 993 | AAEL008137 | hypothetical protein |
| 994 | AAEL008150 | hypothetical protein |
| 995 | AAEL008182 | conserved hypothetical protein |
| 996 | AAEL008184 | conserved hypothetical protein |
| 997 | AAEL008183 | t complex protein |
| 998 | AAEL008185 | conserved hypothetical protein |
| 999 | AAEL008189 | conserved hypothetical protein |
| 1000 | AAEL008220 | conserved hypothetical protein |
| 1001 | AAEL008233 | conserved hypothetical protein |
| 1002 | AAEL008236 | sidestep protein |
| 1003 | AAEL008224 | hypothetical protein |
| 1004 | AAEL008257 | heterogeneous nuclear ribonucleoprotein 27c |
| 1005 | AAEL008256 | cyclin A3, putative |
| 1006 | AAEL001372 | sentrin/sumo-specific protease senp7 |
| 1007 | AAEL008261 | hypothetical protein |
| 1008 | AAEL008320 | conserved hypothetical protein |
| 1009 | AAEL008322 | GPCR Frizzled/Smoothened Family |
| 1010 | AAEL008351 | POSSIBLE INTEGRAL MEMBRANE |
| | | EFFLUX PROTEIN EFPA, putative |
| 1011 | AAEL008356 | hypothetical protein |
| 1012 | AAEL008359 | hypothetical protein |
| 1013 | AAEL015551 | conserved hypothetical protein |
| 1014 | AAEL001399 | conserved hypothetical protein |
| 1015 | AAEL001437 | conserved hypothetical protein |
| 1016 | AAEL001439 | mitochondrial ribosomal protein, L22, putative |
| 1017 | AAEL008379 | P38 mapk |
| 1018 | AAEL008387 | atrial natriuretic peptide receptor |
| 1019 | AAEL008406 | cationic amino acid transporter |
| 1020 | AAEL008421 | cadherin |
| 1021 | AAEL017350 | hypothetical protein |
| 1022 | AAEL008444 | conserved hypothetical protein |
| 1023 | AAEL008461 | surfeit locus protein |
| 1024 | AAEL008476 | conserved hypothetical protein |
| 1025 | AAEL008500 | DEAD box ATP-dependent RNA helicase |
| 1026 | AAEL008503 | hypothetical protein |
| 1027 | AAEL001451 | DNA repair protein Rad62, putative |
| 1028 | AAEL001464 | conserved hypothetical protein |
| 1029 | AAEL008510 | sphingosine kinase a, b |
| 1030 | AAEL008511 | hypothetical protein |
| 1031 | AAEL008555 | conserved hypothetical protein |
| 1032 | AAEL008522 | conserved hypothetical protein |
| 1033 | AAEL008544 | zinc finger protein, putative |
| 1034 | AAEL008537 | transcription factor grauzone, putative |
| 1035 | AAEL008535 | conserved hypothetical protein |
| 1036 | AAEL008521 | conserved hypothetical protein |
| 1037 | AAEL017089 | hypothetical protein |
| 1038 | AAEL008533 | conserved hypothetical protein |
| 1039 | AAEL008547 | conserved hypothetical protein |
| 1040 | AAEL008526 | conserved hypothetical protein |
| 1041 | AAEL008570 | glycoprotein, putative |
| 1042 | AAEL008557 | conserved hypothetical protein |
| 1043 | AAEL008583 | conserved hypothetical protein |
| 1044 | AAEL008580 | conserved hypothetical protein |
| 1045 | AAEL008579 | zinc finger protein |
| 1046 | AAEL015565 | hypothetical protein |
| 1047 | AAEL008602 | conserved hypothetical protein |
| 1048 | AAEL008593 | NAD dependent epimerase/dehydratase |
| 1049 | AAEL008623 | conserved hypothetical protein |
| 1050 | AAEL008638 | cytochrome P450 |
| 1051 | AAEL001490 | acylphosphatase, putative |
| 1052 | AAEL001505 | conserved hypothetical protein |
| 1053 | AAEL001506 | U3 small nucleolar ribonucleoprotein |
| | | protein mpp10 |
| 1054 | AAEL001481 | hypothetical protein |
| 1055 | AAEL015571 | conserved hypothetical protein |
| 1056 | AAEL008678 | conserved hypothetical protein |
| 1057 | AAEL008675 | hypothetical protein |
| 1058 | AAEL008722 | hypothetical protein |
| 1059 | AAEL008724 | conserved hypothetical protein |
| 1060 | AAEL008729 | hypothetical protein |
| 1061 | AAEL008748 | hypothetical protein |
| 1062 | AAEL008759 | hypothetical protein |
| 1063 | AAEL008777 | proto-oncogene tyrosine-protein kinase abl1 |
| 1064 | AAEL008781 | serine-type enodpeptidase, |
| 1065 | AAEL008794 | conserved hypothetical protein |
| 1066 | AAEL008817 | hexamerin 2 beta |
| 1067 | AAEL008822 | conserved hypothetical protein |
| 1068 | AAEL008809 | pickpocket, putative |
| 1069 | AAEL008797 | hypothetical protein |
| 1070 | AAEL001525 | conserved hypothetical protein |
| 1071 | AAEL008839 | hypothetical protein |
| 1072 | AAEL008842 | hypothetical protein |
| 1073 | AAEL008863 | protein regulator of cytokinesis 1 prc1 |
| 1074 | AAEL008868 | conserved hypothetical protein |
| 1075 | AAEL008875 | conserved hypothetical protein |
| 1076 | AAEL008886 | conserved hypothetical protein |
| 1077 | AAEL008884 | hypothetical protein |
| 1078 | AAEL008900 | p15-2a protein, putative |
| 1079 | AAEL008894 | conserved hypothetical protein |
| 1080 | AAEL008915 | sodium-and chloride-activated ATP-sensitive |
| | | potassium channel |
| 1081 | AAEL008923 | ring finger protein |
| 1082 | AAEL001545 | conserved hypothetical protein |
| 1083 | AAEL001585 | predicted protein |
| 1084 | AAEL001595 | conserved hypothetical protein |
| 1085 | AAEL001569 | conserved hypothetical protein |
| 1086 | AAEL001576 | conserved hypothetical protein |
| 1087 | AAEL001588 | glutamate carboxypeptidase |
| 1088 | AAEL001581 | conserved hypothetical protein |
| 1089 | AAEL001559 | conserved hypothetical protein |
| 1090 | AAEL008942 | conserved hypothetical protein |
| 1091 | AAEL008939 | conserved hypothetical protein |
| 1092 | AAEL008983 | adult cuticle protein, putative |
| 1093 | AAEL009022 | adenylate cyclase type |
| 1094 | AAEL009021 | peptidylprolyl isomerase |
| 1095 | AAEL009023 | conserved hypothetical protein |
| 1096 | AAEL009057 | cyclin e |
| 1097 | AAEL009084 | slender lobes, putative |
| 1098 | AAEL009080 | importin 7, |
| 1099 | AAEL001614 | conserved hypothetical protein |
| 1100 | AAEL001622 | dual specificity mitogen-activated protein |
| | | kinase kinase MAPKK |
| 1101 | AAEL001609 | hypothetical protein |
| 1102 | AAEL009147 | conserved hypothetical protein |
| 1103 | AAEL009153 | M-type 9 protein, putative |
| 1104 | AAEL009160 | skp1 |
| 1105 | AAEL009163 | conserved hypothetical protein |
| 1106 | AAEL009167 | bone morphogenetic protein 5/7, bmp5/7 |
| 1107 | AAEL009187 | conserved hypothetical protein |
| 1108 | AAEL009189 | encore protein |
| 1109 | AAEL001663 | hypothetical protein |
| 1110 | AAEL001666 | conserved hypothetical protein |
| 1111 | AAEL001650 | conserved hypothetical protein |
| 1112 | AAEL009241 | translation initiation factor if-2 |
| 1113 | AAEL009233 | zinc metalloprotease |
| 1114 | AAEL009248 | conserved hypothetical protein |
| 1115 | AAEL009260 | conserved hypothetical protein |
| 1116 | AAEL009261 | hypothetical protein |
| 1117 | AAEL009263 | conserved hypothetical protein |
| 1118 | AAEL009262 | hypothetical protein |
| 1119 | AAEL009277 | hypothetical protein |
| 1120 | AAEL009290 | hypothetical protein |
| 1121 | AAEL009296 | histone H3, putative |
| 1122 | AAEL009309 | lipid depleted protein |
| 1123 | AAEL009322 | hypothetical protein |
| 1124 | AAEL001684 | boule protein, putative |
| 1125 | AAEL001700 | hypothetical protein |
| 1126 | AAEL001697 | adenylate cyclase, putative |
| 1127 | AAEL001703 | serine-type enodpeptidase, |
| 1128 | AAEL001691 | adenylate cyclase |
| 1129 | AAEL001693 | serine-type enodpeptidase, |
| 1130 | AAEL009335 | adhesion regulating molecule 1 (110 kda |
| | | cell membrane glycoprotein) |
| 1131 | AAEL009348 | conserved hypothetical protein |
| 1132 | AAEL009393 | conserved hypothetical protein |
| 1133 | AAEL009411 | DNA-binding protein smubp-2 |
| 1134 | AAEL009425 | hypothetical protein |
| 1135 | AAEL009442 | hypothetical protein |
| 1136 | AAEL015633 | conserved hypothetical protein |
| 1137 | AAEL009452 | hypothetical protein |
| 1138 | AAEL009456 | hypothetical protein |
| 1139 | AAEL009454 | conserved hypothetical protein |
| 1140 | AAEL009470 | conserved hypothetical protein |
| 1141 | AAEL009465 | replication factor c/DNA polymerase iii |
| | | gamma-tau subunit |
| 1142 | AAEL009463 | hypothetical protein |
| 1143 | AAEL009484 | conserved hypothetical protein |
| 1144 | AAEL000224 | serine protease |
| 1145 | AAEL000223 | alpha-glucosidase |
| 1146 | AAEL000235 | hypothetical protein |
| 1147 | AAEL001711 | activin receptor type I, putative |
| 1148 | AAEL001727 | hypothetical protein |
| 1149 | AAEL009510 | glucosamine-fructose-6-phosphate |
| | | aminotransferase |
| 1150 | AAEL009500 | conserved hypothetical protein |
| 1151 | AAEL009508 | zinc finger protein |
| 1152 | AAEL017066 | hypothetical protein |
| 1153 | AAEL009518 | timeout/timeless-2 |
| 1154 | AAEL009522 | hypothetical protein |
| 1155 | AAEL009533 | conserved hypothetical protein |
| 1156 | AAEL009551 | Toll-like receptor |
| 1157 | AAEL009576 | conserved hypothetical protein |
| 1158 | AAEL015640 | transcription factor IIIA, putative |
| 1159 | AAEL001745 | candidate tumor suppressor protein |
| 1160 | AAEL001734 | bric-a-brac |
| 1161 | AAEL009586 | hypothetical protein |
| 1162 | AAEL009600 | Ecdysone receptor isoform A Nuclear receptor |
| 1163 | AAEL009602 | conserved hypothetical protein |
| 1164 | AAEL009646 | conserved hypothetical protein |
| 1165 | AAEL009667 | conserved hypothetical protein |
| 1166 | AAEL017306 | hypothetical protein |
| 1167 | AAEL001781 | origin recognition complex subunit |
| 1168 | AAEL001785 | origin recognition complex subunit |
| 1169 | AAEL001788 | hypothetical protein |
| 1170 | AAEL009710 | adhesion regulating molecule 1 (110 kda cell |
| | | membrane glycoprotein) |
| 1171 | AAEL009709 | hypothetical protein |
| 1172 | AAEL009727 | conserved hypothetical protein |
| 1173 | AAEL009716 | hypothetical protein |
| 1174 | AAEL009729 | conserved hypothetical protein |
| 1175 | AAEL009739 | cbl-d |
| 1176 | AAEL009742 | Homeobox protein abdominal-A homolog |
| 1177 | AAEL009755 | conserved hypothetical protein |
| 1178 | AAEL009753 | sodium-dependent phosphate transporter |
| 1179 | AAEL009773 | geminin, putative |
| 1180 | AAEL009772 | conserved hypothetical protein |
| 1181 | AAEL009770 | ubiquitin-conjugating enzyme E2 i |
| 1182 | AAEL009798 | transcription factor IIIA, putative |
| 1183 | AAEL009799 | hypothetical protein |
| 1184 | AAEL001809 | conserved hypothetical protein |
| 1185 | AAEL017094 | hypothetical protein |
| 1186 | AAEL001795 | orfY, putative |
| 1187 | AAEL009854 | conserved hypothetical protein |
| 1188 | AAEL009834 | hypothetical protein |
| 1189 | AAEL009842 | galectin |
| 1190 | AAEL009845 | galectin |
| 1191 | AAEL009836 | conserved hypothetical protein |
| 1192 | AAEL009861 | conserved hypothetical protein |
| 1193 | AAEL009896 | hypothetical protein |
| 1194 | AAEL009894 | leucine-rich immune protein (Coil-less) |
| 1195 | AAEL009886 | kidney-specific Na—K—Cl cotransport protein |
| | | splice isoform A, putative |
| 1196 | AAEL009918 | conserved hypothetical protein |
| 1197 | AAEL009912 | conserved hypothetical protein |
| 1198 | AAEL009935 | conserved hypothetical protein |
| 1199 | AAEL001839 | zinc carboxypeptidase |
| 1200 | AAEL001842 | zinc carboxypeptidase |
| 1201 | AAEL017186 | hypothetical protein |
| 1202 | AAEL001877 | fucosyltransferase 11 (fut11) |
| 1203 | AAEL001858 | hypothetical protein |
| 1204 | AAEL001867 | sodium-dependent phosphate transporter |
| 1205 | AAEL001856 | adenosine kinase |
| 1206 | AAEL009972 | hypothetical protein |
| 1207 | AAEL009962 | hypothetical protein |
| 1208 | AAEL009984 | alanyl-tRNA synthetase |
| 1209 | AAEL009983 | GPCR Frizzled/Smoothened Family |
| 1210 | AAEL009999 | conserved hypothetical protein |
| 1211 | AAEL009998 | conserved hypothetical protein |
| 1212 | AAEL010007 | conserved hypothetical protein |
| 1213 | AAEL010011 | conserved hypothetical protein |
| 1214 | AAEL010001 | conserved hypothetical protein |
| 1215 | AAEL010014 | hypothetical protein |
| 1216 | AAEL010033 | DNA mismatch repair protein pms2 |
| 1217 | AAEL001890 | hypothetical protein |
| 1218 | AAEL001902 | glutamate decarboxylase |
| 1219 | AAEL001908 | hypothetical protein |
| 1220 | AAEL001904 | arp2/3 |
| 1221 | AAEL001900 | lactosylceramide 4-alpha-galactosyltransferase |
| | | (alpha-1,4-galactosyltransferase) |
| 1222 | AAEL010060 | signal recognition particle 68 kda protein |
| 1223 | AAEL010072 | hypothetical protein |
| 1224 | AAEL010080 | origin recognition complex subunit |
| 1225 | AAEL010094 | cyclin b |
| 1226 | AAEL010081 | conserved hypothetical protein |
| 1227 | AAEL010112 | conserved hypothetical protein |
| 1228 | AAEL010097 | conserved hypothetical protein |
| 1229 | AAEL010113 | conserved hypothetical protein |
| 1230 | AAEL010109 | conserved hypothetical protein |
| 1231 | AAEL010117 | fibrinogen and fibronectin |
| 1232 | AAEL010118 | kelch repeat protein |
| 1233 | AAEL010136 | hypothetical protein |
| 1234 | AAEL010155 | hypothetical protein |
| 1235 | AAEL010176 | conserved hypothetical protein |
| 1236 | AAEL010189 | Band 7 protein AAEL010189 |
| 1237 | AAEL001927 | hypothetical protein |
| 1238 | AAEL001917 | ribosome biogenesis protein brix |
| 1239 | AAEL001939 | hypothetical protein |
| 1240 | AAEL001933 | membrane associated ring finger 1, 8 |
| 1241 | AAEL010194 | hypothetical protein |
| 1242 | AAEL010242 | conserved hypothetical protein |
| 1243 | AAEL010226 | daughterless |
| 1244 | AAEL010229 | hypothetical protein |
| 1245 | AAEL010253 | conserved hypothetical protein |
| 1246 | AAEL010244 | abrupt protein |
| 1247 | AAEL010246 | conserved hypothetical protein |
| 1248 | AAEL010249 | conserved hypothetical protein |
| 1249 | AAEL010290 | short-chain dehydrogenase |
| 1250 | AAEL010289 | beta nu integrin subunit |
| 1251 | AAEL010292 | conserved hypothetical protein |
| 1252 | AAEL010294 | membrane-associated guanylate kinase (maguk) |
| 1253 | AAEL015673 | nucleolar complex protein |
| 1254 | AAEL002032 | hypothetical protein |
| 1255 | AAEL002033 | hypothetical protein |
| 1256 | AAEL002010 | conserved hypothetical protein |
| 1257 | AAEL001972 | TATA box binding protein (TBP)-associated |
| | | factor,, putative |
| 1258 | AAEL002006 | conserved hypothetical protein |
| 1259 | AAEL010311 | conserved hypothetical protein |
| 1260 | AAEL010318 | polyadenylate-binding protein |
| 1261 | AAEL010309 | hypothetical protein |
| 1262 | AAEL010319 | heat shock transcription factor (hsf) |
| 1263 | AAEL010343 | aryl hydrocarbon receptor nuclear translocator |
| | | (arnt protein) (hypoxia-inducible factor 1 beta) |
| 1264 | AAEL017368 | hypothetical protein |
| 1265 | AAEL010378 | conserved hypothetical protein |
| 1266 | AAEL010381 | glucosyl/glucuronosyl transferases |
| 1267 | AAEL010370 | aldehyde oxidase |
| 1268 | AAEL010420 | hypothetical protein |
| 1269 | AAEL010422 | replication-associated histone mRNA stem |
| | | loop-binding protein, putative |
| 1270 | AAEL010417 | conserved hypothetical protein |
| 1271 | AAEL010434 | Vitellogenin-A1 Precursor (VG)(PVG1) |
| | | [Contains Vitellin light chain(VL); |
| | | Vitellin heavy chain(VH)] |
| 1272 | AAEL010437 | heparan n-sulfatase |
| 1273 | AAEL010447 | hypothetical protein |
| 1274 | AAEL010446 | protein phosphatase 2c |
| 1275 | AAEL010454 | hypothetical protein |
| 1276 | AAEL010473 | NAD dependent epimerase/dehydratase |
| 1277 | AAEL002079 | TATA binding protein, putative |
| 1278 | AAEL002054 | hypothetical protein |
| 1279 | AAEL002058 | hypothetical protein |
| 1280 | AAEL002063 | cationic amino acid transporter |
| 1281 | AAEL010501 | zinc finger protein |
| 1282 | AAEL010490 | hypothetical protein |
| 1283 | AAEL010495 | hypothetical protein |
| 1284 | AAEL010509 | bridging integrator |
| 1285 | AAEL010503 | hypothetical protein |
| 1286 | AAEL010507 | hypothetical protein |
| 1287 | AAEL010510 | conserved hypothetical protein |
| 1288 | AAEL010538 | conserved hypothetical protein |
| 1289 | AAEL010546 | heat shock factor binding protein, putative |
| 1290 | AAEL010562 | hypothetical protein |
| 1291 | AAEL010587 | conserved hypothetical protein |
| 1292 | AAEL010588 | striatin, putative |
| 1293 | AAEL010578 | mixed-lineage leukemia protein, mll |
| 1294 | AAEL002128 | serine protease |
| 1295 | AAEL010631 | conserved hypothetical protein |
| 1296 | AAEL010623 | conserved hypothetical protein |
| 1297 | AAEL010627 | conserved hypothetical protein |
| 1298 | AAEL010638 | histone H1, putative |
| 1299 | AAEL010644 | ribonucleoside-diphosphate reductase large chain |
| 1300 | AAEL010664 | actin binding protein, putative |
| 1301 | AAEL010670 | lethal(2)essential for life protein, l2efl |
| 1302 | AAEL010679 | monocyte to macrophage differentiation protein |
| 1303 | AAEL010665 | developmentally regulated RNA-binding protein |
| 1304 | AAEL010674 | hypothetical protein |
| 1305 | AAEL010660 | alpha-B-crystallin, putative |
| 1306 | AAEL017191 | hypothetical protein |
| 1307 | AAEL010692 | OCP-II protein, putative |
| 1308 | AAEL010708 | hypothetical protein |
| 1309 | AAEL010709 | hypothetical protein |
| 1310 | AAEL000289 | conserved hypothetical protein |
| 1311 | AAEL000272 | conserved hypothetical protein |
| 1312 | AAEL000260 | conserved hypothetical protein |
| 1313 | AAEL002136 | hypothetical protein |
| 1314 | AAEL017571 | hypothetical protein |
| 1315 | AAEL010715 | hypothetical protein |
| 1316 | AAEL017338 | hypothetical protein |
| 1317 | AAEL010755 | hypothetical protein |
| 1318 | AAEL010748 | hypothetical protein |
| 1319 | AAEL010766 | inositol triphosphate 3-kinase c |
| 1320 | AAEL010784 | conserved hypothetical protein |
| 1321 | AAEL002184 | F-actin capping protein beta subunit |
| 1322 | AAEL002181 | cuticle protein, putative |
| 1323 | AAEL002219 | zinc finger protein, putative |
| 1324 | AAEL010829 | protein arginine n-methyltransferase |
| 1325 | AAEL010808 | conserved hypothetical protein |
| 1326 | AAEL010827 | programmed cell death protein 11 (pre-rRNA |
| | | processing protein rrp5) |
| 1327 | AAEL010841 | lupus la ribonucleoprotein |
| 1328 | AAEL010855 | cdc6 |
| 1329 | AAEL010877 | conserved hypothetical protein |
| 1330 | AAEL010901 | mannose binding lectin, putative |
| 1331 | AAEL010904 | rothmund-thomson syndrome DNA helicase |
| | | recq4 |
| 1332 | AAEL010907 | conserved hypothetical protein |
| 1333 | AAEL010908 | hypothetical protein |
| 1334 | AAEL010912 | dipeptidyl-peptidase |
| 1335 | AAEL002250 | terminal deoxycytidyl transferase rev1 |
| 1336 | AAEL010930 | l-asparaginase |
| 1337 | AAEL010945 | conserved hypothetical protein |
| 1338 | AAEL010965 | cubulin |
| 1339 | AAEL002303 | conserved hypothetical protein |
| 1340 | AAEL002319 | hypothetical protein |
| 1341 | AAEL002320 | hypothetical protein |
| 1342 | AAEL002307 | leucine-rich transmembrane protein |
| 1343 | AAEL011003 | hypothetical protein |
| 1344 | AAEL011013 | single-minded |
| 1345 | AAEL011043 | conserved hypothetical protein |
| 1346 | AAEL011062 | hypothetical protein |
| 1347 | AAEL002332 | hypothetical protein |
| 1348 | AAEL002354 | heme peroxidase |
| 1349 | AAEL011085 | conserved hypothetical protein |
| 1350 | AAEL011124 | PHD finger protein |
| 1351 | AAEL011138 | hypothetical protein |
| 1352 | AAEL011145 | ribosomal protein S6 kinase, 90 kD, polypeptide |
| 1353 | AAEL011161 | conserved hypothetical protein |
| 1354 | AAEL011173 | conserved hypothetical protein |
| 1355 | AAEL011172 | conserved hypothetical protein |
| 1356 | AAEL011175 | alkaline phosphatase |
| 1357 | AAEL011176 | hypothetical protein |
| 1358 | AAEL011178 | posterior sex combs protein |
| 1359 | AAEL002403 | hypothetical protein |
| 1360 | AAEL002376 | hypothetical protein |
| 1361 | AAEL002375 | NBP2b protein, putative |
| 1362 | AAEL011199 | conserved hypothetical protein |
| 1363 | AAEL011215 | F-box and WD40 domain protein 7 (fbw7) |
| 1364 | AAEL002423 | conserved hypothetical protein |
| 1365 | AAEL002417 | troponin t, invertebrate |
| 1366 | AAEL002429 | hypothetical protein |
| 1367 | AAEL011248 | innexin |
| 1368 | AAEL011253 | rho-GTPase-activating protein |
| 1369 | AAEL011264 | phosphatidylethanolamine-binding protein |
| 1370 | AAEL011276 | mitochondrial glutamate carrier protein |
| 1371 | AAEL011280 | voltage-dependent p/q type calcium channel |
| 1372 | AAEL011291 | protease m1 zinc metalloprotease |
| 1373 | AAEL011298 | hypothetical protein |
| 1374 | AAEL011303 | cell division protein ftsj |
| 1375 | AAEL011313 | epoxide hydrolase |
| 1376 | AAEL011330 | conserved hypothetical protein |
| 1377 | AAEL011326 | conserved hypothetical protein |
| 1378 | AAEL002453 | conserved hypothetical protein |
| 1379 | AAEL002442 | conserved hypothetical protein |
| 1380 | AAEL002443 | conserved hypothetical protein |
| 1381 | AAEL002454 | conserved hypothetical protein |
| 1382 | AAEL011358 | origin recognition complex subunit |
| 1383 | AAEL011357 | maintenance of killer 16 (mak16) protein |
| 1384 | AAEL011362 | hypothetical protein |
| 1385 | AAEL011400 | conserved hypothetical protein |
| 1386 | AAEL011422 | conserved hypothetical protein |
| 1387 | AAEL002473 | hypothetical protein |
| 1388 | AAEL002462 | hypothetical protein |
| 1389 | AAEL002477 | hypothetical protein |
| 1390 | AAEL017575 | hypothetical protein |
| 1391 | AAEL011473 | chromatin regulatory protein sir2 |
| 1392 | AAEL011498 | copper-zinc (Cu—Zn) superoxide dismutase |
| 1393 | AAEL011496 | chitinase |
| 1394 | AAEL016971 | hypothetical protein |
| 1395 | AAEL011516 | conserved hypothetical protein |
| 1396 | AAEL011515 | hypothetical protein |
| 1397 | AAEL011520 | sucrose transport protein |
| 1398 | AAEL011536 | phosphoglucomutase |
| 1399 | AAEL011527 | eukaryotic translation initiation factor |
| 1400 | AAEL011533 | hypothetical protein |
| 1401 | AAEL011532 | hypothetical protein |
| 1402 | AAEL011537 | hypothetical protein |
| 1403 | AAEL002529 | conserved hypothetical protein |
| 1404 | AAEL002503 | yippee protein |
| 1405 | AAEL002522 | adenosine deaminase acting on RNA (adar)-2 |
| 1406 | AAEL002497 | hypothetical protein |
| 1407 | AAEL011586 | hypothetical protein |
| 1408 | AAEL011592 | secreted mucin MUC17, putative |
| 1409 | AAEL011598 | hypothetical protein |
| 1410 | AAEL011596 | mitotic checkpoint serine/threonine-protein |
| | | kinase bub1 and bubr1 |
| 1411 | AAEL011597 | conserved hypothetical protein |
| 1412 | AAEL011615 | rab gdp/GTP exchange factor |
| 1413 | AAEL011633 | fibrinogen and fibronectin |
| 1414 | AAEL011631 | hypothetical protein |
| 1415 | AAEL011640 | hypothetical protein |
| 1416 | AAEL011635 | conserved hypothetical protein |
| 1417 | AAEL011648 | cyclin d |
| 1418 | AAEL011655 | aspartyl-tRNA synthetase |
| 1419 | AAEL011664 | conserved hypothetical protein |
| 1420 | AAEL011653 | thyroid hormone receptor interactor |
| 1421 | AAEL000322 | hypothetical protein |
| 1422 | AAEL000352 | hypothetical protein |
| 1423 | AAEL017203 | hypothetical protein |
| 1424 | AAEL000356 | cysteine-rich venom protein, putative |
| 1425 | AAEL000302 | cysteine-rich venom protein, putative |
| 1426 | AAEL000375 | cysteine-rich venom protein, putative |
| 1427 | AAEL000317 | cysteine-rich venom protein, putative |
| 1428 | AAEL000348 | conserved hypothetical protein |
| 1429 | AAEL000327 | Ecdysone-induced protein 78C Nuclear receptor |
| 1430 | AAEL000324 | tyrosine-protein kinase drl |
| 1431 | AAEL002557 | cationic amino acid transporter |
| 1432 | AAEL002541 | cystinosin |
| 1433 | AAEL002569 | serine/threonine kinase |
| 1434 | AAEL011696 | conserved hypothetical protein |
| 1435 | AAEL011686 | starch branching enzyme ii |
| 1436 | AAEL011695 | guanine nucleotide exchange factor |
| 1437 | AAEL011712 | diacylglycerol kinase, alpha, beta, gamma |
| 1438 | AAEL011726 | hypothetical protein |
| 1439 | AAEL011735 | conserved hypothetical protein |
| 1440 | AAEL011743 | hypothetical protein |
| 1441 | AAEL011754 | conserved hypothetical protein |
| 1442 | AAEL011765 | conserved hypothetical protein |
| 1443 | AAEL011767 | hypothetical protein |
| 1444 | AAEL011761 | cytochrome P450 |
| 1445 | AAEL011769 | cytochrome P450 |
| 1446 | AAEL011780 | DNA mismatch repair protein muts |
| 1447 | AAEL011809 | glucose dehydrogenase |
| 1448 | AAEL011811 | DNA replication licensing factor MCM3 |
| 1449 | AAEL002652 | hypothetical protein |
| 1450 | AAEL002635 | conserved hypothetical protein |
| 1451 | AAEL011846 | hypothetical protein |
| 1452 | AAEL011852 | hypothetical protein |
| 1453 | AAEL011859 | conserved hypothetical protein |
| 1454 | AAEL011862 | conserved hypothetical protein |
| 1455 | AAEL011868 | conserved hypothetical protein |
| 1456 | AAEL011870 | rap55 |
| 1457 | AAEL011875 | conserved hypothetical protein |
| 1458 | AAEL011872 | conserved hypothetical protein |
| 1459 | AAEL011892 | receptor for activated C kinase, putative |
| 1460 | AAEL011895 | odorant receptor |
| 1461 | AAEL017451 | hypothetical protein |
| 1462 | AAEL017179 | hypothetical protein |
| 1463 | AAEL017200 | hypothetical protein |
| 1464 | AAEL011958 | conserved hypothetical protein |
| 1465 | AAEL011979 | calmodulin |
| 1466 | AAEL017253 | hypothetical protein |
| 1467 | AAEL002690 | beat protein |
| 1468 | AAEL002681 | Vanin-like protein 1 precursor, putative |
| 1469 | AAEL011989 | signal peptide peptidase |
| 1470 | AAEL012014 | l-lactate dehydrogenase |
| 1471 | AAEL012020 | conserved hypothetical protein |
| 1472 | AAEL012013 | hypothetical protein |
| 1473 | AAEL012015 | DEAD box ATP-dependent RNA helicase |
| 1474 | AAEL012012 | conserved hypothetical protein |
| 1475 | AAEL012032 | hypothetical protein |
| 1476 | AAEL012028 | proacrosin, putative |
| 1477 | AAEL012030 | preproacrosin, putative |
| 1478 | AAEL012041 | sulphate transporter |
| 1479 | AAEL012055 | dfg10 protein |
| 1480 | AAEL012057 | enhancer of polycomb |
| 1481 | AAEL002719 | conserved hypothetical protein |
| 1482 | AAEL002700 | conserved hypothetical protein |
| 1483 | AAEL012130 | ordml, arthropod |
| 1484 | AAEL012142 | timeout/timeless-2 |
| 1485 | AAEL012137 | conserved hypothetical protein |
| 1486 | AAEL012141 | odorant receptor |
| 1487 | AAEL012155 | conserved hypothetical protein |
| 1488 | AAEL012164 | spaetzle-like cytokine |
| 1489 | AAEL012209 | ring finger protein |
| 1490 | AAEL017392 | hypothetical protein |
| 1491 | AAEL012214 | hypothetical protein |
| 1492 | AAEL012273 | conserved hypothetical protein |
| 1493 | AAEL012279 | Eukaryotic translation initiation factor 3 |
| | | subunit J (eIF3j) |
| 1494 | AAEL012288 | sugar transporter |
| 1495 | AAEL012295 | conserved hypothetical protein |
| 1496 | AAEL012293 | conserved hypothetical protein |
| 1497 | AAEL012300 | conserved hypothetical protein |
| 1498 | AAEL012312 | proliferation-associated 2g4 (pa2g4/ebp1) |
| 1499 | AAEL012314 | conserved hypothetical protein |
| 1500 | AAEL002793 | conserved hypothetical protein |
| 1501 | AAEL002785 | DNA polymerase epsilon subunit b |
| 1502 | AAEL002811 | conserved hypothetical protein |
| 1503 | AAEL002814 | hypothetical protein |
| 1504 | AAEL002810 | DNA replication licensing factor MCM5 |
| 1505 | AAEL002774 | slender lobes, putative |
| 1506 | AAEL012378 | serine-type protease inhibitor |
| 1507 | AAEL012392 | conserved hypothetical protein |
| 1508 | AAEL012388 | predicted protein |
| 1509 | AAEL002843 | conserved hypothetical protein |
| 1510 | AAEL002836 | carbon catabolite repressor protein |
| 1511 | AAEL002848 | tubulin beta chain |
| 1512 | AAEL002849 | zinc finger protein, putative |
| 1513 | AAEL012418 | deoxyribonuclease ii |
| 1514 | AAEL012427 | conserved hypothetical protein |
| 1515 | AAEL012430 | AMP dependent ligase |
| 1516 | AAEL012437 | hypothetical protein |
| 1517 | AAEL012441 | conserved hypothetical protein |
| 1518 | AAEL012458 | hypothetical protein |
| 1519 | AAEL012461 | monocarboxylate transporter |
| 1520 | AAEL012455 | alcohol dehydrogenase |
| 1521 | AAEL000402 | conserved hypothetical protein |
| 1522 | AAEL002867 | phenylalanyl-tRNA synthetase alpha chain |
| 1523 | AAEL002856 | conserved hypothetical protein |
| 1524 | AAEL002863 | zinc finger protein |
| 1525 | AAEL002855 | hypothetical protein |
| 1526 | AAEL002882 | conserved hypothetical protein |
| 1527 | AAEL002872 | cytochrome P450 |
| 1528 | AAEL012473 | vav1 |
| 1529 | AAEL012480 | sodium/calcium exchanger |
| 1530 | AAEL012499 | histone H2A |
| 1531 | AAEL012504 | hypothetical protein |
| 1532 | AAEL012526 | hypothetical protein |
| 1533 | AAEL012527 | conserved hypothetical protein |
| 1534 | AAEL012546 | DNA replication licensing factor MCM6 |
| 1535 | AAEL002889 | hypothetical protein |
| 1536 | AAEL002884 | hypothetical protein |
| 1537 | AAEL002905 | conserved hypothetical protein |
| 1538 | AAEL012566 | conserved hypothetical protein |
| 1539 | AAEL012586 | conserved hypothetical protein |
| 1540 | AAEL012600 | hypothetical protein |
| 1541 | AAEL012610 | conserved hypothetical protein |
| 1542 | AAEL012618 | conserved hypothetical protein |
| 1543 | AAEL012629 | deoxyuridine 5′-triphosphate nucleotidohydrolase |
| 1544 | AAEL002932 | conserved hypothetical protein |
| 1545 | AAEL002942 | hypothetical protein |
| 1546 | AAEL002947 | AMP dependent ligase |
| 1547 | AAEL012644 | conserved hypothetical protein |
| 1548 | AAEL012647 | conserved hypothetical protein |
| 1549 | AAEL012650 | conserved hypothetical protein |
| 1550 | AAEL012658 | rgs-gaip interacting protein gipc |
| 1551 | AAEL012684 | conserved hypothetical protein |
| 1552 | AAEL012682 | hypothetical protein |
| 1553 | AAEL012676 | conserved hypothetical protein |
| 1554 | AAEL012708 | conserved hypothetical protein |
| 1555 | AAEL012714 | hypothetical protein |
| 1556 | AAEL017254 | hypothetical protein |
| 1557 | AAEL002949 | Osiris, putative |
| 1558 | AAEL002958 | conserved hypothetical protein |
| 1559 | AAEL002991 | hypothetical protein |
| 1560 | AAEL003003 | glutamate-gated chloride channel |
| 1561 | AAEL002989 | hypothetical protein |
| 1562 | AAEL012811 | mitochondrial peptide chain release factor |
| 1563 | AAEL012810 | conserved hypothetical protein |
| 1564 | AAEL012802 | conserved hypothetical protein |
| 1565 | AAEL012812 | exosome complex exonuclease RRP41, putative |
| 1566 | AAEL012830 | anti-silencing protein |
| 1567 | AAEL012832 | cytochrome B561 |
| 1568 | AAEL012836 | cytochrome B561 |
| 1569 | AAEL012838 | conserved hypothetical protein |
| 1570 | AAEL012876 | conserved hypothetical protein |
| 1571 | AAEL012875 | snare protein sec22 |
| 1572 | AAEL003058 | glucosyl/glucuronosyl transferases |
| 1573 | AAEL003051 | conserved hypothetical protein |
| 1574 | AAEL012927 | hypothetical protein |
| 1575 | AAEL012927 | hypothetical protein |
| 1576 | AAEL012960 | importin alpha |
| 1577 | AAEL012979 | conserved hypothetical protein |
| 1578 | AAEL017272 | GPCR Serotonin Family |
| 1579 | AAEL012996 | rho guanine dissociation factor |
| 1580 | AAEL013004 | conserved hypothetical protein |
| 1581 | AAEL013024 | hypothetical protein |
| 1582 | AAEL003112 | conserved hypothetical protein |
| 1583 | AAEL013036 | conserved hypothetical protein |
| 1584 | AAEL013037 | conserved hypothetical protein |
| 1585 | AAEL013038 | hypothetical protein |
| 1586 | AAEL013051 | conserved hypothetical protein |
| 1587 | AAEL013054 | conserved hypothetical protein |
| 1588 | AAEL013045 | exosome complex exonuclease RRP41, putative |
| 1589 | AAEL013078 | glycosyltransferase |
| 1590 | AAEL013091 | hypothetical protein |
| 1591 | AAEL017424 | hypothetical protein |
| 1592 | AAEL003130 | bcr-associated protein, bap |
| 1593 | AAEL013112 | Peptidoglycan Recognition Protein (Long) |
| 1594 | AAEL013110 | conserved hypothetical protein |
| 1595 | AAEL013149 | conserved hypothetical protein |
| 1596 | AAEL013156 | hypothetical protein |
| 1597 | AAEL013148 | predicted protein |
| 1598 | AAEL013154 | hypothetical protein |
| 1599 | AAEL013160 | GPCR Frizzled/Smoothened Family |
| 1600 | AAEL013168 | arrowhead |
| 1601 | AAEL000470 | hypothetical protein |
| 1602 | AAEL000426 | hypothetical protein |
| 1603 | AAEL003172 | transcription factor IIIA, putative |
| 1604 | AAEL003158 | conserved hypothetical protein |
| 1605 | AAEL003168 | hypothetical protein |
| 1606 | AAEL013174 | conserved hypothetical protein |
| 1607 | AAEL013179 | 8-oxoguanine DNA glycosylase |
| 1608 | AAEL013190 | gustatory receptor Gr22 |
| 1609 | AAEL013212 | prefoldin, subunit, putative |
| 1610 | AAEL013216 | conserved hypothetical protein |
| 1611 | AAEL013226 | conserved hypothetical protein |
| 1612 | AAEL003186 | hypothetical protein |
| 1613 | AAEL003207 | hypothetical protein |
| 1614 | AAEL013240 | conserved hypothetical protein |
| 1615 | AAEL013248 | hypothetical protein |
| 1616 | AAEL013252 | hypothetical protein |
| 1617 | AAEL013249 | hypothetical protein |
| 1618 | AAEL013251 | hypothetical protein |
| 1619 | AAEL013291 | conserved hypothetical protein |
| 1620 | AAEL013288 | conserved hypothetical protein |
| 1621 | AAEL013285 | hypothetical protein |
| 1622 | AAEL003210 | tetraspanin 29fa |
| 1623 | AAEL003214 | salivary gland growth factor |
| 1624 | AAEL003267 | hypothetical protein |
| 1625 | AAEL013311 | hypothetical protein |
| 1626 | AAEL013312 | dual-specificity protein phosphatase, putative |
| 1627 | AAEL013325 | conserved hypothetical protein |
| 1628 | AAEL013344 | lethal(2)essential for life protein, l2efl |
| 1629 | AAEL013338 | lethal(2)essential for life protein, l2efl |
| 1630 | AAEL003312 | hypothetical protein |
| 1631 | AAEL003321 | hypothetical protein |
| 1632 | AAEL003285 | translocation associated membrane protein |
| 1633 | AAEL013400 | DEAD box ATP-dependent RNA helicase |
| 1634 | AAEL017652 | 18S_rRNA |
| 1635 | AAEL003355 | conserved hypothetical protein |
| 1636 | AAEL003343 | hypothetical protein |
| 1637 | AAEL003346 | heparan sulphate 2-o-sulfotransferase |
| 1638 | AAEL003327 | zinc finger protein |
| 1639 | AAEL013412 | conserved hypothetical protein |
| 1640 | AAEL013453 | sarcolemmal associated protein, putative |
| 1641 | AAEL013463 | nucleolar protein 10 |
| 1642 | AAEL017276 | hypothetical protein |
| 1643 | AAEL003377 | signal recognition particle |
| 1644 | AAEL003382 | Ro ribonucleoprotein autoantigen, putative |
| 1645 | AAEL013465 | conserved hypothetical protein |
| 1646 | AAEL013471 | hypothetical protein |
| 1647 | AAEL013490 | conserved hypothetical protein |
| 1648 | AAEL003435 | conserved hypothetical protein |
| 1649 | AAEL003404 | hypothetical protein |
| 1650 | AAEL013510 | smaug protein |
| 1651 | AAEL013521 | tryptophanyl-tRNA synthetase |
| 1652 | AAEL013539 | SH2/SH3 adaptor protein |
| 1653 | AAEL013546 | estrogen-related receptor (ERR) |
| 1654 | AAEL013562 | zinc finger protein, putative |
| 1655 | AAEL003454 | phocein protein, putative |
| 1656 | AAEL013564 | conserved hypothetical protein |
| 1657 | AAEL013569 | conserved hypothetical protein |
| 1658 | AAEL013593 | hypothetical protein |
| 1659 | AAEL003494 | goodpasture antigen-binding protein |
| 1660 | AAEL003493 | GDI interacting protein, putative |
| 1661 | AAEL003502 | hypothetical protein |
| 1662 | AAEL003507 | Toll-like receptor |
| 1663 | AAEL013601 | short-chain dehydrogenase |
| 1664 | AAEL013608 | sugar transporter |
| 1665 | AAEL013616 | hypothetical protein |
| 1666 | AAEL013635 | conserved hypothetical protein |
| 1667 | AAEL003544 | conserved hypothetical protein |
| 1668 | AAEL017367 | hypothetical protein |
| 1669 | AAEL003542 | conserved hypothetical protein |
| 1670 | AAEL003547 | hypothetical protein |
| 1671 | AAEL013653 | tata-box binding protein |
| 1672 | AAEL017342 | hypothetical protein |
| 1673 | AAEL013690 | DNA mismatch repair protein pms2 |
| 1674 | AAEL000487 | hypothetical protein |
| 1675 | AAEL000500 | conserved hypothetical protein |
| 1676 | AAEL003554 | leucine rich repeat protein |
| 1677 | AAEL013701 | meiotic recombination protein spo11 |
| 1678 | AAEL013724 | conserved hypothetical protein |
| 1679 | AAEL013726 | epsin 4/enthoprotin |
| 1680 | AAEL017141 | hypothetical protein |
| 1681 | AAEL013733 | Protein distal antenna |
| 1682 | AAEL013734 | hypothetical protein |
| 1683 | AAEL013738 | hypothetical protein |
| 1684 | AAEL003574 | hypothetical protein |
| 1685 | AAEL003571 | factor for adipocyte differentiation |
| 1686 | AAEL013761 | ADP-ribosylation factor, arf |
| 1687 | AAEL013778 | F-actin capping protein alpha |
| 1688 | AAEL013784 | hypothetical protein |
| 1689 | AAEL017560 | hypothetical protein |
| 1690 | AAEL003595 | protein serine/threonine kinase, putative |
| 1691 | AAEL013789 | conserved hypothetical protein |
| 1692 | AAEL013796 | conserved hypothetical protein |
| 1693 | AAEL013799 | hypothetical protein |
| 1694 | AAEL013805 | conserved hypothetical protein |
| 1695 | AAEL013806 | conserved hypothetical protein |
| 1696 | AAEL013809 | conserved hypothetical protein |
| 1697 | AAEL013813 | conserved hypothetical protein |
| 1698 | AAEL013830 | bmp-induced factor |
| 1699 | AAEL013832 | Homeobox protein abdominal-A homolog |
| 1700 | AAEL013838 | hypothetical protein |
| 1701 | AAEL003646 | conserved hypothetical protein |
| 1702 | AAEL003663 | hypothetical protein |
| 1703 | AAEL003688 | conserved hypothetical protein |
| 1704 | AAEL015684 | hypothetical protein |
| 1705 | AAEL003657 | zinc finger protein |
| 1706 | AAEL013852 | conserved hypothetical protein |
| 1707 | AAEL013850 | conserved hypothetical protein |
| 1708 | AAEL013860 | hypothetical protein |
| 1709 | AAEL013872 | hypothetical protein |
| 1710 | AAEL013896 | smad4 |
| 1711 | AAEL003767 | hypothetical protein |
| 1712 | AAEL013940 | chromatin assembly factor i P60 subunit |
| 1713 | AAEL013955 | conserved hypothetical protein |
| 1714 | AAEL003797 | hypothetical protein |
| 1715 | AAEL003804 | conserved hypothetical protein |
| 1716 | AAEL003775 | hypothetical protein |
| 1717 | AAEL003793 | hypothetical protein |
| 1718 | AAEL003791 | conserved hypothetical protein |
| 1719 | AAEL003792 | conserved hypothetical protein |
| 1720 | AAEL003807 | conserved hypothetical protein |
| 1721 | AAEL013958 | NBP2b protein, putative |
| 1722 | AAEL013968 | conserved hypothetical protein |
| 1723 | AAEL013965 | conserved hypothetical protein |
| 1724 | AAEL013975 | transcription factor IIIA, putative |
| 1725 | AAEL013989 | protein translocation complex beta subunit, |
| | | putative |
| 1726 | AAEL013998 | conserved hypothetical protein |
| 1727 | AAEL014001 | yellow protein precursor, putative |
| 1728 | AAEL003817 | kappa b-ras |
| 1729 | AAEL003824 | conserved hypothetical protein |
| 1730 | AAEL003861 | bmp-induced factor |
| 1731 | AAEL014020 | hypothetical protein |
| 1732 | AAEL014025 | cell division cycle 20 (cdc20) (fizzy) |
| 1733 | AAEL014033 | conserved hypothetical protein |
| 1734 | AAEL014036 | hypothetical protein |
| 1735 | AAEL014047 | hypothetical protein |
| 1736 | AAEL014055 | thymidine kinase |
| 1737 | AAEL014583 | 60S acidic ribosomal protein P2 |
| 1738 | AAEL005722 | 60S ribosomal protein L7a |
| 1739 | AAEL017931 | U1 spliceosomal RNA |
| 1740 | AAEL017646 | U1 spliceosomal RNA |
| 1741 | AAEL005266 | 40S ribosomal protein S14 |
| 1742 | AAEL004175 | 40S ribosomal protein S17 |
| 1743 | AAEL014562 | 60S ribosomal protein L12 |
| 1744 | AAEL006785 | 60S ribosomal protein L18a |
| 1745 | AAEL007715 | 60S ribosomal protein L21 |
| 1746 | AAEL007771 | 60S ribosomal protein L22 |
| 1747 | AAEL005817 | 60S ribosomal protein L26 |
| 1748 | AAEL006698 | 60S ribosomal protein L31 |
| 1749 | AAEL003942 | 60S ribosomal protein L44 L41 |
| 1750 | AAEL000987 | 60S ribosomal protein L8 |
| 1751 | AAEL007699 | 60S ribosomal protein L9 |
| 1752 | AAEL006511 | anopheles stephensi ubiquitin |
| 1753 | AAEL007698 | AUB |
| 1754 | AAEL005097 | cold induced protein (BnC24A) |
| 1755 | AAEL004851 | |
| 1756 | AAEL011424 | histone H3 |
| 1757 | AAEL000529 | hypothetical protein |
| 1758 | AAEL004060 | hypothetical protein |
| 1759 | AAEL004151 | hypothetical protein |
| 1760 | AAEL004249 | hypothetical protein |
| 1761 | AAEL004503 | hypothetical protein |
| 1762 | AAEL005451 | hypothetical protein |
| 1763 | AAEL001274 | hypothetical protein |
| 1764 | AAEL000766 | |
| 1765 | AAEL008969 | |
| 1766 | AAEL008994 | |
| 1767 | AAEL009151 | |
| 1768 | AAEL009185 | |
| 1769 | AAEL017468 | |
| 1770 | AAEL009188 | |
| 1771 | AAEL009201 | |
| 1772 | AAEL001673 | |
| 1773 | AAEL009341 | |
| 1774 | AAEL009403 | |
| 1775 | AAEL009496 | |
| 1776 | AAEL009825 | |
| 1777 | AAEL017413 | |
| 1778 | AAEL010299 | |
| 1779 | AAEL002047 | |
| 1780 | AAEL010573 | |
| 1781 | AAEL002372 | |
| 1782 | AAEL017231 | |
| 1783 | AAEL011447 | |
| 1784 | AAEL011471 | |
| 1785 | AAEL011504 | |
| 1786 | AAEL011587 | |
| 1787 | AAEL011656 | |
| 1788 | AAEL002639 | |
| 1789 | AAEL002832 | |
| 1790 | AAEL002881 | |
| 1791 | AAEL012944 | |
| 1792 | AAEL013221 | |
| 1793 | AAEL013272 | |
| 1794 | AAEL003396 | |
| 1795 | AAEL013533 | |
| 1796 | AAEL013536 | |
| 1797 | AAEL003582 | |
| 1798 | AAEL005027 | |
| 1799 | AAEL017536 | |
| 1800 | AAEL008353 | |
| 1801 | AAEL017198 | |
| 1802 | AAEL016995 | |
| 1803 | AAEL017590 | Ref Transcript AaegL3.1_AAEL017868-RA |
| 1804 | AAEL017868 | Ref Transcript AaegL3.1_AAEL017868-RA |
| 1805 | AAEL005629 | ribosomal protein L35 |
| 1806 | AAEL000010 | ribosomal protein L36 |
| 1807 | AAEL004325 | ribosomal protein L5 |
| 1808 | AAEL000068 | ribosomal protein S25 |
| 1809 | AAEL007824 | ribosomal protein S29 |
| 1810 | AAEL008297 | Sodium channel |
| 1811 | AAEL016638 | tRNA |
| 1812 | AAEL017826 | U1 spliceosomal RNA |
| 1813 | AAEL017609 | U1 spliceosomal RNA |
| 1826 | AAEL010379 | P-glycoprotein (PgP) |
| 1827 | AAEL007823 | Argonaute-3 |
| 1828 | JF924909.1 | Cytochrome p450 (CYP9J26) |

TABLE 3
(male sterility)

| Seq ID | Gene Symbol |
|---|---|
| 166 | AAEL000442 |
| 167 | AAEL000888 |
| 168 | AAEL001371 |
| 169 | AAEL002079 |
| 170 | AAEL003077 |
| 171 | AAEL004266 |
| 172 | AAEL004492 |
| 173 | AAEL004517 |
| 174 | AAEL004651 |
| 175 | AAEL004933 |
| 176 | AAEL005232 |
| 177 | AAEL007609 |
| 178 | AAEL008182 |
| 179 | AAEL008605 |
| 180 | AAEL009383 |
| 181 | AAEL010737 |
| 182 | AAEL011339 |
| 183 | AAEL011380 |
| 184 | AAEL011433 |
| 185 | AAEL012330 |
| 186 | AAEL012340 |
| 187 | AAEL012341 |
| 188 | AAEL012344 |
| 189 | AAEL012345 |
| 190 | AAEL012349 |
| 191 | AAEL012350 |
| 192 | AAEL012706 |
| 193 | AAEL012710 |
| 194 | AAEL012715 |
| 195 | AAEL014031 |
| 196 | AAEL014218 |
| 197 | AAEL014238 |
| 198 | AAEL014339 |
| 199 | AAEL014904 |
| 200 | AAEL014916 |
| 201 | AAEL014920 |
| 202 | AAEL014921 |
| 203 | AAEL015390 |
| 204 | AGAP000005 |
| 205 | AGAP000005 |
| 206 | AGAP000005 |
| 207 | AGAP000306 |
| 208 | AGAP000306 |
| 209 | AGAP000306 |
| 210 | AGAP000670 |
| 211 | AGAP000670 |
| 212 | AGAP000670 |
| 213 | AGAP001652 |
| 214 | AGAP001879 |
| 215 | AGAP001879 |
| 216 | AGAP001879 |
| 217 | AGAP002353 |
| 218 | AGAP002872 |
| 219 | AGAP002872 |
| 220 | AGAP002872 |
| 221 | AGAP003500 |
| 222 | AGAP003501 |
| 223 | AGAP003519 |
| 224 | AGAP003519 |
| 225 | AGAP003519 |
| 226 | AGAP003545 |
| 227 | AGAP003545 |
| 228 | AGAP003545 |
| 229 | AGAP003796 |
| 230 | AGAP004096 |
| 231 | AGAP004096 |
| 232 | AGAP004096 |
| 233 | AGAP004840 |
| 234 | AGAP004840 |
| 235 | AGAP004840 |
| 236 | AGAP005130 |
| 237 | AGAP005130 |
| 238 | AGAP005130 |
| 239 | AGAP005733 |
| 240 | AGAP005733 |
| 241 | AGAP005733 |
| 242 | AGAP006237 |
| 243 | AGAP006237 |
| 244 | AGAP006237 |
| 245 | AGAP007242 |
| 246 | AGAP007242 |
| 247 | AGAP007242 |
| 248 | AGAP008084 |
| 249 | AGAP008084 |
| 250 | AGAP008084 |
| 251 | AGAP008374 |
| 252 | AGAP008374 |
| 253 | AGAP008374 |
| 254 | AGAP008642 |
| 255 | AGAP008642 |
| 256 | AGAP008642 |
| 257 | AGAP009091 |
| 258 | AGAP009091 |
| 259 | AGAP009091 |
| 260 | AGAP009442 |
| 261 | AGAP009442 |
| 262 | AGAP009442 |
| 263 | AGAP010909 |
| 264 | AGAP010909 |
| 265 | AGAP010909 |
| 266 | AGAP010958 |
| 267 | AGAP012380 |
| 268 | AGAP012380 |
| 269 | AGAP012380 |
| 270 | CPIJ000025 |
| 271 | CPIJ000367 |
| 272 | CPIJ001133 |
| 273 | CPIJ001692 |
| 274 | CPIJ001739 |
| 275 | CPIJ001883 |
| 276 | CPIJ002710 |
| 277 | CPIJ002715 |
| 278 | CPIJ002718 |
| 279 | CPIJ002719 |
| 280 | CPIJ002726 |
| 281 | CPIJ002789 |
| 282 | CPIJ005348 |
| 283 | CPIJ005588 |
| 284 | CPIJ006105 |
| 285 | CPIJ007600 |
| 286 | CPIJ008100 |
| 287 | CPIJ008391 |
| 288 | CPIJ008494 |
| 289 | CPIJ008983 |
| 290 | CPIJ013307 |
| 291 | CPIJ013432 |
| 292 | CPIJ014043 |
| 293 | CPIJ014354 |
| 294 | CPIJ014659 |
| 295 | CPIJ014870 |
| 296 | CPIJ015607 |
| 297 | CPIJ015663 |
| 298 | CPIJ015791 |
| 299 | CPIJ018368 |
| 300 | CPIJ019419 |
| 301 | CPIJ019949 |

TABLE 4
(male sterility)

| Seq ID | Gene Symbol |
|---|---|
| 302 | AAEL001340 |
| 303 | AAEL001606 |
| 304 | AAEL002425 |
| 305 | AAEL002792 |
| 306 | AAEL003660 |
| 307 | AAEL004696 |
| 308 | AAEL004974 |
| 309 | AAEL006254 |
| 310 | AAEL006488 |
| 311 | AAEL006492 |
| 312 | AAEL008042 |
| 313 | AAEL008587 |
| 314 | AAEL008844 |
| 315 | AAEL008924 |
| 316 | AAEL008958 |
| 317 | AAEL009114 |
| 318 | AAEL009174 |
| 319 | AAEL009340 |
| 320 | AAEL009969 |
| 321 | AAEL010565 |
| 322 | AAEL010789 |
| 323 | AAEL010792 |
| 324 | AAEL011474 |
| 325 | AAEL011478 |
| 326 | AAEL011663 |
| 327 | AAEL011757 |
| 328 | AAEL011921 |
| 329 | AAEL014330 |
| 330 | AGAP000460 |
| 331 | AGAP000460 |
| 332 | AGAP000460 |
| 333 | AGAP000471 |
| 334 | AGAP000471 |
| 335 | AGAP000471 |
| 336 | AGAP000662 |
| 337 | AGAP000662 |
| 338 | AGAP000662 |
| 339 | AGAP001177 |
| 340 | AGAP001177 |
| 341 | AGAP001177 |
| 342 | AGAP001179 |
| 343 | AGAP001179 |
| 344 | AGAP001179 |
| 345 | AGAP001271 |
| 346 | AGAP001271 |
| 347 | AGAP001271 |
| 348 | AGAP001278 |
| 349 | AGAP001278 |
| 350 | AGAP001278 |
| 351 | AGAP001293 |
| 352 | AGAP001293 |
| 353 | AGAP001293 |
| 354 | AGAP001335 |
| 355 | AGAP001335 |
| 356 | AGAP001335 |
| 357 | AGAP001337 |
| 358 | AGAP001337 |
| 359 | AGAP001337 |
| 360 | AGAP001339 |
| 361 | AGAP001339 |
| 362 | AGAP001339 |
| 363 | AGAP001367 |
| 364 | AGAP001367 |
| 365 | AGAP001367 |
| 366 | AGAP001388 |
| 367 | AGAP001388 |
| 368 | AGAP001388 |
| 369 | AGAP001463 |
| 370 | AGAP001463 |
| 371 | AGAP001463 |
| 372 | AGAP001478 |
| 373 | AGAP001478 |
| 374 | AGAP001478 |
| 375 | AGAP001481 |
| 376 | AGAP001481 |
| 377 | AGAP001481 |
| 378 | AGAP001498 |
| 379 | AGAP001498 |
| 380 | AGAP001498 |
| 381 | AGAP002471 |
| 382 | AGAP002471 |
| 383 | AGAP002471 |
| 384 | AGAF002801 |
| 385 | AGAP004050 |
| 386 | AGAP004416 |
| 387 | AGAP004416 |
| 388 | AGAP004416 |
| 389 | AGAP004645 |
| 390 | AGAP004930 |
| 391 | AGAP006887 |
| 392 | AGAP006887 |
| 393 | AGAP006887 |
| 394 | AGAP007963 |
| 395 | AGAP008806 |
| 396 | CPIJ001185 |
| 397 | CPIJ001186 |
| 398 | CPIJ001187 |
| 399 | CPIJ001560 |
| 400 | CPIJ003158 |
| 401 | CPIJ003766 |
| 402 | CPIJ004057 |
| 403 | CPIJ004058 |
| 404 | CPIJ004318 |
| 405 | CPIJ005975 |
| 406 | CPIJ005976 |
| 407 | CPIJ007071 |
| 408 | CPIJ007072 |
| 409 | CPIJ007101 |
| 410 | CPIJ007172 |
| 411 | CPIJ007789 |
| 412 | CPIJ008481 |
| 413 | CPIJ008673 |
| 414 | CPIJ009011 |
| 415 | CPIJ009270 |
| 416 | CPIJ011557 |
| 417 | CPIJ011558 |
| 418 | CPIJ011708 |
| 419 | CPIJ012810 |
| 420 | CPIJ013126 |
| 421 | CPIJ015620 |
| 422 | CPIJ015622 |
| 423 | CPIJ017065 |
| 424 | CPIJ017887 |
| 425 | CPIJ019248 |
| 426 | CPIJ019249 |
| 427 | FBgn0127180 |

TABLE 5
(female sterility)

| SEQ ID | Name or access | |
|---|---|---|
| NO. | number | Description |
| 1829 | AeSCP-2 | Aedes aegypti sterol carrier protein-2 |
| | (AF510492.1) | |
| 1830 | AeAct-4 | Aedes aegypti pupal-specific flight |
| | (AY531222.2) | muscle actin mRNA, complete cds. |
| 1831 | AAEL002000 | Aedes aegypti zinc carboxypeptidase |
| | | partial mRNA |
| 1832 | AAEL005747 | Aedes aegypti hypothetical protein |
| | | partial mRNA (testicle target) |
| 700 | AAEL005656 | Aedes aegypti AAEL005656-RA partial |
| | | mRNA. |
| 678 | AAEL017015 | Aedes aegypti AAEL017015-RA mRNA |
| 652 | AAEL005212 | Aedes aegypti AAEL005212-RA mRNA. |
| 733 | AAEL005922 | Aedes aegypti AAEL005922-RA mRNA. |
| | | Aedes aegypti AAEL005922-RB partial |
| | | mRNA. |
| 722 | AAEL000903 | Aedes aegypti AAEL000903-RA |
| | | (ENY2_AEDAE), mRNA. |
| 638 | AAEL005049 | Aedes aegypti AAEL005049-RA mRNA. |
| 1753 | AAEL007698 | PIWI protein (Aub) |
| 1827 | AAEL007823 | PIWI protein (AGO3) |

As used herein, the term “downregulates an expression” or “downregulating expression” refers to causing, directly or indirectly, reduction in the transcription of a desired gene (as described herein), reduction in the amount, stability or translatability of transcription products (e.g. RNA) of the gene, and/or reduction in translation of the polypeptide(s) encoded by the desired gene.

Downregulating expression of a pathogen resistance gene product of a mosquito can be monitored, for example, by direct detection of gene transcripts (for example, by PCR), by detection of polypeptide(s) encoded by the gene (for example, by Western blot or immunoprecipitation), by detection of biological activity of polypeptides encode by the gene (for example, catalytic activity, ligand binding, and the like), or by monitoring changes in the mosquitoes (for example, reduced motility of the mosquito etc). Additionally or alternatively downregulating expression of a pathogen resistance gene product may be monitored by measuring pathogen levels (e.g. viral levels, bacterial levels etc.) in the mosquitoes as compared to wild type (i.e. control) mosquitoes not treated by the agents of the invention.

According to a specific embodiment the nucleic acid larvicide downregulates (reduces expression of) the target gene by at least 20%, 30%, 40%, 50%, or more, say 60%, 70%, 80%, 90% or more even 100%, as compared to the expression of the same target gene in an untreated control in the same species and developmental stage.

In some embodiments of the invention, the nucleic acid agent is a double stranded RNA (dsRNA). As used herein the term “dsRNA” relates to two strands of anti-parallel polyribonucleic acids held together by base pairing. The two strands can be of identical length or of different lengths provided there is enough sequence homology between the two strands that a double stranded structure is formed with at least 80%, 90%, 95% or 100% complementarity over the entire length. According to an embodiment of the invention, there are no overhangs for the dsRNA molecule. According to another embodiment of the invention, the dsRNA molecule comprises overhangs. According to other embodiments, the strands are aligned such that there are at least 1, 2, or 3 bases at the end of the strands which do not align (i.e., for which no complementary bases occur in the opposing strand) such that an overhang of 1, 2 or 3 residues occurs at one or both ends of the duplex when strands are annealed.

It will be noted that the dsRNA can be defined in terms of the nucleic acid sequence of the DNA encoding the target gene transcript, and it is understood that a dsRNA sequence corresponding to the coding sequence of a gene comprises an RNA complement of the gene's coding sequence, or other sequence of the gene which is transcribed into RNA.

The inhibitory RNA sequence can be greater than 90% identical, or even 100% identical, to the portion of the target gene transcript. Alternatively, the duplex region of the RNA may be defined functionally as a nucleotide sequence that is capable of hybridizing with a portion of the target gene transcript under stringent conditions (e.g., 400 mM NaCl, 40 mM PIPES pH 6.4, 1 mM EDTA, 60 degrees C. hybridization for 12-16 hours; followed by washing). The length of the double-stranded nucleotide sequences complementary to the target gene transcript may be at least about 18, 19, 21, 25, 50, 100, 200, 300, 400, 491, 500, 550, 600, 650, 700, 750, 800, 900, 1000 or more bases. In some embodiments of the invention, the length of the double-stranded nucleotide sequence is approximately from about 18 to about 1000, about 18 to about 750, about 18 to about 510, about 18 to about 400, about 18 to about 250 nucleotides in length.

The term “corresponds to” as used herein means a polynucleotide sequence homologous to all or a portion of a reference polynucleotide sequence. In contradistinction, the term “complementary to” is used herein to mean that the complementary sequence is homologous to all or a portion of a reference polynucleotide sequence. For example, the nucleotide sequence “TATAC” corresponds to a reference sequence “TATAC” and is complementary to a reference sequence “GTATA”.

The present teachings relate to various lengths of dsRNA, whereby the shorter version i.e., x is shorter or equals 50 bp (e.g., 17-50), is referred to as siRNA or miRNA. Longer dsRNA molecules of 51-600 are referred to herein as dsRNA, which can be further processed for siRNA molecules. According to some embodiments, the nucleic acid sequence of the dsRNA is greater than 15 base pairs in length. According to yet other embodiments, the nucleic acid sequence of the dsRNA is 19-25 base pairs in length, 30-100 base pairs in length, 100-250 base pairs in length or 100-500 base pairs in length. According to still other embodiments, the dsRNA is 500-800 base pairs in length, 700-800 base pairs in length, 300-600 base pairs in length, 350-500 base pairs in length or 400-450 base pairs in length. In some embodiments, the dsRNA is 400 base pairs in length. In some embodiments, the dsRNA is 750 base pairs in length.

The term “siRNA” refers to small inhibitory RNA duplexes (generally between 17-30 basepairs, but also longer e.g., 31-50 bp) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is theorized to result from providing Dicer with a substrate (27mer) instead of a product (21mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-UUCAAGAGA-3′ (Brummelkamp, T. R. et al. (2002) Science 296: 550, SEQ ID NO: 428) and 5′-UUUGUGUAG-3′ (Castanotto, D. et al. (2002) RNA 8:1454, SEQ ID NO: 429). It will be recognized by one of skill in the art that the resulting single chain oligonucleotide forms a stem-loop or hairpin structure comprising a double-stranded region capable of interacting with the RNAi machinery.

As used herein, the phrase “microRNA (also referred to herein interchangeably as “miRNA” or “miR”) or a precursor thereof” refers to a microRNA (miRNA) molecule acting as a post-transcriptional regulator. Typically, the miRNA molecules are RNA molecules of about 20 to 22 nucleotides in length which can be loaded into a RISC complex and which direct the cleavage of another RNA molecule, wherein the other RNA molecule comprises a nucleotide sequence essentially complementary to the nucleotide sequence of the miRNA molecule.

Typically, a miRNA molecule is processed from a “pre-miRNA” or as used herein a precursor of a pre-miRNA molecule by proteins, such as DCL proteins, present in any plant cell and loaded onto a RISC complex where it can guide the cleavage of the target RNA molecules.

Pre-microRNA molecules are typically processed from pri-microRNA molecules (primary transcripts). The single stranded RNA segments flanking the pre-microRNA are important for processing of the pri-miRNA into the pre-miRNA. The cleavage site appears to be determined by the distance from the stem-ssRNA junction (Han et al. 2006, Cell 125, 887-901, 887-901).

As used herein, a “pre-miRNA” molecule is an RNA molecule of about 100 to about 200 nucleotides, preferably about 100 to about 130 nucleotides which can adopt a secondary structure comprising an imperfect double stranded RNA stem and a single stranded RNA loop (also referred to as “hairpin”) and further comprising the nucleotide sequence of the miRNA (and its complement sequence) in the double stranded RNA stem. According to a specific embodiment, the miRNA and its complement are located about 10 to about 20 nucleotides from the free ends of the miRNA double stranded RNA stem. The length and sequence of the single stranded loop region are not critical and may vary considerably, e.g. between 30 and 50 nucleotides in length. The complementarity between the miRNA and its complement need not be perfect and about 1 to 3 bulges of unpaired nucleotides can be tolerated. The secondary structure adopted by an RNA molecule can be predicted by computer algorithms conventional in the art such as mFOLD. The particular strand of the double stranded RNA stem from the pre-miRNA which is released by DCL activity and loaded onto the RISC complex is determined by the degree of complementarity at the 5′ end, whereby the strand which at its 5′ end is the least involved in hydrogen bounding between the nucleotides of the different strands of the cleaved dsRNA stem is loaded onto the RISC complex and will determine the sequence specificity of the target RNA molecule degradation. However, if empirically the miRNA molecule from a particular synthetic pre-miRNA molecule is not functional (because the “wrong” strand is loaded on the RISC complex), it will be immediately evident that this problem can be solved by exchanging the position of the miRNA molecule and its complement on the respective strands of the dsRNA stem of the pre-miRNA molecule. As is known in the art, binding between A and U involving two hydrogen bounds, or G and U involving two hydrogen bounds is less strong that between G and C involving three hydrogen bounds.

Naturally occurring miRNA molecules may be comprised within their naturally occurring pre-miRNA molecules but they can also be introduced into existing pre-miRNA molecule scaffolds by exchanging the nucleotide sequence of the miRNA molecule normally processed from such existing pre-miRNA molecule for the nucleotide sequence of another miRNA of interest. The scaffold of the pre-miRNA can also be completely synthetic. Likewise, synthetic miRNA molecules may be comprised within, and processed from, existing pre-miRNA molecule scaffolds or synthetic pre-miRNA scaffolds. Some pre-miRNA scaffolds may be preferred over others for their efficiency to be correctly processed into the designed microRNAs, particularly when expressed as a chimeric gene wherein other DNA regions, such as untranslated leader sequences or transcription termination and polyadenylation regions are incorporated in the primary transcript in addition to the pre-microRNA.

According to the present teachings, the dsRNA molecules may be naturally occurring or synthetic.

The dsRNA can be a mixture of long and short dsRNA molecules such as, dsRNA, siRNA, siRNA+dsRNA, siRNA+miRNA, or a combination of same.

The nucleic acid larvicide is designed for specifically targeting a target gene of interest. It will be appreciated that the nucleic acid larvicide can be used to downregulate one or more target genes (e.g., belonging to groups (i) to (iv), as described above). If a number of target genes are targeted, a heterogenic composition which comprises a plurality of nucleic acid larvicides for targeting a number of target genes is used. Alternatively the plurality of nucleic acid larvicides are separately formulated. According to a specific embodiment, a number of distinct nucleic acid larvicide molecules for a single target are used, which may be separately or simultaneously (i.e., co-formulation) applied.

For example, in order to silence the expression of an mRNA of interest, synthesis of the dsRNA suitable for use with some embodiments of the invention can be selected as follows. First, the mRNA sequence is scanned including the 3′ UTR and the 5′ UTR.

Second, the mRNA sequence is compared to an appropriate genomic database using any sequence alignment software, such as the BLAST software available from the NCBI server (wwwdotncbidotnlmdotnihdotgov/BLAST/). Putative regions in the mRNA sequence which exhibit significant homology to other coding sequences are filtered out.

Qualifying target sequences are selected as template for dsRNA synthesis. Preferred sequences are those that have as little homology to other genes in the genome to reduce an “off-target” effect.

It will be appreciated that the RNA silencing agent of some embodiments of the invention need not be limited to those molecules containing only RNA, but further encompasses chemically-modified nucleotides and non-nucleotides.

According to one embodiment, the dsRNA specifically targets a gene selected from the group consisting of sodium channel (AAEL008297), P-glycoprotein (AAEL010379), Argonaute-3 (AAEL007823), cytochrome p450 (CYP9J26, JF924909.1), Aub (AAEL007698), AeSCP-2 (AF510492.1), AeAct-4 (AY531222.2), AAEL002000, AAEL005747, AAEL017015, AAEL005212, AAEL005922, AAEL000903, AAEL005656 or AAEL005049.

Thus, a combination of two or more silencing agents e.g., dsRNAs, for a single target gene or distinct genes is contemplated according to the present teachings.

Thus, for example, a combination of dsRNA targeting the genes Aubergine (Aub, AAEL007698) and Argonaute-3 (AAEL007823) is contemplated herein. When referring to targeting together it is understood that the larvae may be administered two silencing agents, e.g., dsRNAs, concomitantly or subsequently to one another (e.g. hours or days apart).

According to one embodiment, the dsRNA is selected from the group consisting of SEQ ID NOs: 1822-1825 and 1857-1868.

According to a specific embodiment, the dsRNA comprises SEQ ID NOs: 1858 and 1832.

The dsRNA may be synthesized using any method known in the art, including either enzymatic syntheses or solid-phase syntheses. These are especially useful in the case of short polynucleotide sequences with or without modifications as explained above. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example: Sambrook, J. and Russell, D. W. (2001), “Molecular Cloning: A Laboratory Manual”; Ausubel, R. M. et al., eds. (1994, 1989), “Current Protocols in Molecular Biology,” Volumes I-III, John Wiley & Sons, Baltimore, Md.; Perbal, B. (1988), “A Practical Guide to Molecular Cloning,” John Wiley & Sons, New York; and Gait, M. J., ed. (1984), “Oligonucleotide Synthesis”; utilizing solid-phase chemistry, e.g. cyanoethyl phosphoramidite followed by deprotection, desalting, and purification by, for example, an automated trityl-on method or HPLC.

According to a specific embodiment, large scale dsRNA preparation is performed by PCR using synthetic DNA templates, such as with the Ambion® MEGAscript® RNAi Kit. dsRNA integrity is verified on gel and purified by a column based method. The concentration of the dsRNA is evaluated both by Nano-drop and gel-based estimation. This dsRNA serves for the following experiments.

According to a specific embodiment, the cell is devoid of a heterologous promoter for driving recombinant expression of the dsRNA (exogenous), rendering the nucleic acid molecule of the instant invention a naked molecule. The nucleic acid agent may still comprise modifications that may affect its stability and bioavailability (e.g., PNA).

The term “recombinant expression” refers to an expression from a nucleic acid construct.

As used herein “devoid of a heterologous promoter for driving expression of the dsRNA” means that the cell doesn't include a cis-acting regulatory sequence (e.g., heterologous) transcribing the dsRNA in the cell. As used herein the term “heterologous” refers to exogenous, not-naturally occurring within the native cell (such as by position of integration, or being non-naturally found within the cell).

Although the instant teachings mainly concentrate on the use of dsRNA which is not comprised in or transcribed from an expression vector (naked), the present teachings also contemplate an embodiment wherein the nucleic acid larvicide is ligated into a nucleic acid construct comprising additional regulatory elements. Thus, according to some embodiments of the invention there is provided a nucleic acid construct comprising an isolated nucleic acid agent comprising a nucleic acid sequence larvicide.

For transcription from an expression cassette, a regulatory region (e.g., promoter, enhancer, silencer, leader, intron and polyadenylation) may be used to modulate the transcription of the RNA strand (or strands). Therefore, in one embodiment, there is provided a nucleic acid construct comprising the nucleic acid larvicide. The nucleic acid construct can have polynucleotide sequences constructed to facilitate transcription of the RNA molecules of the present invention are operably linked to one or more promoter sequences functional in a host cell. The polynucleotide sequences may be placed under the control of an endogenous promoter normally present in the host genome. The polynucleotide sequences of the present invention, under the control of an operably linked promoter sequence, may further be flanked by additional sequences that advantageously affect its transcription and/or the stability of a resulting transcript. Such sequences are generally located upstream of the promoter and/or downstream of the 3′ end of the expression construct. The term “operably linked”, as used in reference to a regulatory sequence and a structural nucleotide sequence, means that the regulatory sequence causes regulated expression of the linked structural nucleotide sequence. “Regulatory sequences” or “control elements” refer to nucleotide sequences located upstream, within, or downstream of a structural nucleotide sequence, and which influence the timing and level or amount of transcription, RNA processing or stability, or translation of the associated structural nucleotide sequence. Regulatory sequences may include promoters, translation leader sequences, introns, enhancers, stem-loop structures, repressor binding sequences, termination sequences, pausing sequences, polyadenylation recognition sequences, and the like. In some embodiments, the host is an algae, and promoter and other regulatory elements are active in algae.

As mentioned, the composition-of matter of some embodiments comprises cells, which comprises the nucleic acid larvicide.

As used herein the term “cell” or “cells” refers to a mosquito larvae ingestible cell.

Examples of such cells include, but are not limited to, cells of phytoplankton (e.g., algae), fungi (e.g., Legendium giganteum), bacteria, and zooplankton such as rotifers.

Specific examples include, bacteria (e.g., cocci and rods), filamentous algae and detritus.

The choice of the cell may depend on the target larvae.

Analyzing the gut content of mosquitoes and larvae may be used to elucidate their preferred diet. The skilled artisan knows how to characterize the gut content. Typically the gut content is stained such as by using a fluorochromatic stain, 4′,6-diamidino-2-phenylindole or DAPI.

Cells (also referred to herein as “host cells”) of particular interest are the prokaryotes and the lower eukaryotes, such as fungi. Illustrative prokaryotes, both Gram-negative and -positive, include Enterobacteriaceae; Bacillaceae; Rhizobiceae; Spirillaceae; Lactobacillaceae; and phylloplane organisms such as members of the Pseudomonadaceae.

An exemplary list includes Bacillus spp., including B. megaterium, B. subtilis; B. cereus, Bacillus thuringiensis, Escherichia spp., including E. coli, and/or Pseudomonas spp., including P. cepacia, P. aeruginosa, and P. fluorescens. Among eukaryotes are fungi, such as Phycomycetes and Ascomycetes, which includes yeast, such as Schizosaccharomyces; and Basidiomycetes, Rhodotorula, Aureobasidium, Sporobolomyces, Saccharomyces spp., and Sporobolomyces spp.

According to a specific embodiment, the host cell is an algal cell.

Various algal species can be used in accordance with the teachings of the invention since they are a significant part of the diet for many kinds of mosquito larvae that feed opportunistically on microorganisms as well as on small aquatic animals such as rotifers.

Examples of algae that can be used in accordance with the present teachings include, but are not limited to, blue-green algae as well as green algae.

According to a specific embodiment, the algal cell is a cyanobacterium cell which is in itself toxic to mosquitoes as taught by Marten 2007 Biorational Control of Mosquitoes. American mosquito control association Bulletin No. 7.

Specific examples of algal cells which can be used in accordance with the present teachings are provided in Marten, G. G. (1986) Mosquito control by plankton management: the potential of indigestible green algae. Journal of Tropical Medicine and Hygiene, 89: 213-222, and further listed infra.

### Green Algae

Actinastrum hantzschii
Ankistrodesmus falcatus
Ankistrodesmus spiralis
Aphanochaete elegans

### Chlamydomonas sp.

Chlorella ellipsoidea
Chlorella pyrenoidosa
Chlorella variegata
Chlorococcum hypnosporum
Chodatella brevispina
Closterium acerosum
Closteriopsis acicularis
Coccochloris peniocystis
Crucigenia lauterbornii
Crucigenia tetrapedia
Coronastrum ellipsoideum
Cosmarium botrytis
Desmidium swartzii
Eudorina elegans
Gloeocystis gigas
Golenkinia minutissima
Gonium multicoccum
Nannochloris oculata
Oocystis mars sonii
Oocystis minuta
Oocystis pusilla
Palmella texensis
Pandorina morum
Paulschulzia pseudovolvox
Pediastrum clathratum
Pediastrum duplex
Pediastrum simplex
Planktosphaeria gelatinosa
Polyedriopsis spinulosa
Pseudococcomyxa adhaerans
Quadrigula closterioides
Radiococcus nimbatus
Scenedesmus basiliensis
Spirogyra pratensis
Staurastrum gladiosum
Tetraedron bitridens
Trochiscia hystrix

### Blue-Green Algae

Anabaena catenula
Anabaena spiroides
Chroococcus turgidus
Cylindrospermum licheniforme

### Bucapsis sp. (U. Texas No. 1519)

Lyngbya spiralis
Microcystis aeruginosa
Nodularia spumigena
Nostoc linckia
Oscillatoria lutea

### Phormidiumfaveolarum

Spinilina platensis

### Other

Compsopogon coeruleus
CTyptomonas ovata
Navicula pelliculosa

The nucleic acid larvicide is introduced into the cells. To this end cells are typically selected exhibiting natural competence or are rendered competent, also referred to as artificial competence.

Competence is the ability of a cell to take up nucleic acid molecules e.g., the nucleic acid larvicide, from its environment.

A number of methods are known in the art to induce artificial competence.

Thus, artificial competence can be induced in laboratory procedures that involve making the cell passively permeable to the nucleic acid larvicide by exposing it to conditions that do not normally occur in nature. Typically the cells are incubated in a solution containing divalent cations (e.g., calcium chloride) under cold conditions, before being exposed to a heat pulse (heat shock).

Electroporation is another method of promoting competence. In this method the cells are briefly shocked with an electric field (e.g., 10-20 kV/cm) which is thought to create holes in the cell membrane through which the nucleic acid larvicide may enter. After the electric shock the holes are rapidly closed by the cell's membrane-repair mechanisms.

Yet alternatively or additionally, cells may be treated with enzymes to degrade their cell walls, yielding. These cells are very fragile but take up foreign nucleic acids at a high rate.

Exposing intact cells to alkali cations such as those of cesium or lithium allows the cells to take up nucleic acids. Improved protocols use this transformation method, while employing lithium acetate, polyethylene glycol, and single-stranded nucleic acids. In these protocols, the single-stranded molecule preferentially binds to the cell wall in yeast cells, preventing double stranded molecule from doing so and leaving it available for transformation.

Enzymatic digestion or agitation with glass beads may also be used to transform cells.

Particle bombardment, microprojectile bombardment, or biolistics is yet another method for artificial competence. Particles of gold or tungsten are coated with the nucleic acid agent and then shot into cells.

Astier C R Acad Sci Hebd Seances Acad Sci D. 1976 Feb. 23; 282(8):795-7, which is hereby incorporated by reference in its entirety, teaches transformation of a unicellular, facultative chemoheterotroph blue-green Algae, Aphanocapsa 6714. The recipient strain becomes competent when the growth reaches its second, slower, exponential phase.

Vázquez-Acevedo M¹Mitochondrion. 2014 Feb. 21. pii: 51567-7249(14)00019-1. doi: 10.1016/j.mito.2014.02.005, which is hereby incorporated by reference in its entirety, teaches transformation of algal cells e.g., Chlamydomonas reinhardtii, Polytomella sp. and Volvox carteri by generating import-competent mitochondria.

According to a specific embodiment the composition of the invention comprises an RNA binding protein.

According to a specific embodiment, the dsRNA binding protein (DRBP) comprises any of the family of eukaryotic, prokaryotic, and viral-encoded products that share a common evolutionarily conserved motif specifically facilitating interaction with dsRNA. Polypeptides which comprise dsRNA binding domains (DRBDs) may interact with at least 11 bp of dsRNA, an event that is independent of nucleotide sequence arrangement. More than 20 DRBPs have been identified and reportedly function in a diverse range of critically important roles in the cell. Examples include the dsRNA-dependent protein kinase PKR that functions in dsRNA signaling and host defense against virus infection and DICER.

Alternatively or additionally, an siRNA binding protein may be used as taught in U.S. Pat. Application No. 20140045914, which is herein incorporated by reference in its entirety.

According to a specific embodiment the RNA binding protein is the p19 RNA binding protein. The protein may increase in vivo stability of an siRNA molecule by coupling it at a binding site where the homodimer of the p19 RNA binding proteins is formed and thus protecting the siRNA from external attacks and accordingly, it can be utilized as an effective siRNA delivery vehicle.

According to a specific embodiment, the target-oriented peptide is located on the surface of the siRNA binding protein.

According to specific embodiments of the invention, whole cell preparations, cell extracts, cell suspensions, cell homogenates, cell lysates, cell supernatants, cell filtrates, or cell pellets of cell cultures of cells comprising the nucleic acid larvicide can be used.

For feeding adult mosquitoes, the cells or may be further combined with food supplements which are typically consumed by adult mosquitoes.

Adult mosquitoes typically feed on blood (female mosquitoes) and nectar of flowers (male mosquitoes), but have been known to ingest non-natural feeds as well. Mosquitoes can be fed various foodstuffs including but not limited to egg/soy protein mixture, carbohydrate foods such as sugar solutions (e.g. sugar syrup), corn syrup, honey, various fruit juices, raisins, apple slices and bananas. These can be provided as a dry mix or as a solution in open feeders. Soaked cotton balls, sponges or alike can also be used to providing a solution (e.g. sugar solution) to adult mosquitoes.

Feed suitable for adult mosquitoes may further include blood, blood components (e.g. plasma, hemoglobin, gamma globulin, red blood cells, adenosine triphosphate, glucose, and cholesterol), or an artificial medium (e.g., such a media is disclosed in U.S. Pat. No. 8,133,524 and in U.S. Patent Application No. 20120145081, both of which are incorporated by reference herein). The composition of some embodiments of the invention may further comprise at least one of a surface-active agent, an inert carrier vehicle, a preservative, a humectant, a feeding stimulant, an attractant, an encapsulating agent, a binder, an emulsifier, a dye, an ultra-violet protector, a buffer, a flow agent or fertilizer, micronutrient donors, or other preparations that influence the growth of the plant.

Additionally, the composition may be supplemented with larval food (food bait) or with excrements of farm animals, on which the larvae feed.

According to one embodiment, the composition is administered to the larvae by feeding.

Feeding the larva with the composition can be effected for about 2 hours to 120 hours, about 2 hours to 108 hours, about 2 hours to 96 hours, about 2 hours to 84 hours, about 2 hours to 72 hours, for about 2 hours to 60 hours, about 2 hours to 48 hours, about 2 hours to 36 hours, about 2 hours to 24 hours, about 2 hours to 12 hours, 12 hours to 24 hours, about 24 hours to 36 hours, about 24 hours to 48 hours, about 36 hours to 48 hours, for about 48 hours to 60 hours, about 60 hours to 72 hours, about 72 hours to 84 hours, about 84 hours to 96 hours, about 96 hours to 108 hours, or about 108 hours to 120 hours.

According to a specific embodiment, the composition is administered to the larvae by feeding for 48-96 hours.

According to one embodiment, feeding the larva with the composition is affected until the larva reaches pupa stage.

According to one embodiment, prior to feeding the larva with dsRNA, the larvae are first soaked with dsRNA.

Soaking the larva with the composition can be effected for about 2 hours to 96 hours, about 2 hours to 84 hours, about 2 hours to 72 hours, for about 2 hours to 60 hours, about 2 hours to 48 hours, about 2 hours to 36 hours, about 2 hours to 24 hours, about 2 hours to 12 hours, 12 hours to 96 hours, about 12 hours to 84 hours, about 12 hours to 72 hours, for about 12 hours to 60 hours, about 12 hours to 48 hours, about 12 hours to 36 hours, about 12 hours to 24 hours, or about 24 hours to 48 hours.

According to a specific embodiment, the composition is administered to the larvae by soaking for 12-24 hours.

Thus, for example, larvae (e.g. first, second, third or four instar larva, e.g. third instar larvae) are first treated (in groups of about 100 larvae) with dsRNA at a dose of about 0.001-5 μg/μL (e.g. 0.2 μg/μL), in a final volume of about 3 mL of dsRNA solution in autoclaved water. After soaking in the dsRNA solutions for about 12-48 hours (e.g. for 24 hrs) at 25-29° C. (e.g. 27° C.), the larvae are transferred into containers so as not to exceed concentration of about 200-500 larvae/1500 mL (e.g. 300 larvae/1500 mL) of chlorine-free tap water, and provided with food containing dsRNA (e.g. agarose cubes containing 300 μg of dsRNA, e.g. 1 μg of dsRNA/larvae). The larva are fed once a day until they reach pupa stage (e.g. for 2-5 days, e.g. four days). Larvae are also fed with additional food requirements, e.g. 2-10 mg/100 mL (e.g. 6 mg/100 mL) lab dog/cat diet suspended in water.

Feeding the larva can be effected using any method known in the art. Thus, for example, the larva may be fed with agrose cubes, chitosan nanoparticles, oral delivery or diet containing dsRNA.

Chitosan nanoparticles: A group of 15-20 3rd-instar mosquito larvae are transferred into a container (e.g. 500 ml glass beaker) containing 50-1000 ml, e.g. 100 ml, of deionized water. One sixth of the gel slices that are prepared from dsRNA (e.g. 32 μg of dsRNA) are added into each beaker. Approximately an equal amount of the gel slices are used to feed the larvae once a day for a total of 2-5 days, e.g. four days (see Insect Mol Biol. 2010 19(5):683-93).

Oral delivery of dsRNA: First instar larvae (less than 24 hrs old) are treated in groups of 10-100, e.g. 50, in a final volume of 25-100 μl of dsRNA, e.g. 75 μl of dsRNA, at various concentrations (ranging from 0.01 to 5 μg/μl, e.g. 0.02 to 0.5 μg/μl-dsRNAs) in tubes e.g. 2 mL microfuge tube (see J Insect Sci. 2013; 13:69).

Diet containing dsRNA: larvae are fed a single concentration of 1-2000 ng dsRNA/mL, e.g. 1000 ng dsRNA/mL, diet in a diet overlay bioassay for a period of 1-10 days, e.g. 5 days (see PLoS One. 2012; 7(10): e47534.).

Diet containing dsRNA: Newly emerged larvae are starved for 1-12 hours, e.g. 2 hours, and are then fed with a single drop of 0.5-10 e.g. 1 containing 1-20 μg, e.g. 4 μg, dsRNA (1-20 μg of dsRNA/larva, e.g. 4 μg of dsRNA/larva) (see Appl Environ Microbiol. 2013 August; 79(15):4543-50).

According to a further specific embodiment, the composition may further comprise a chemical larvicide, a biochemical larvicide (a biopesticide) or a combination of same.

According to the U.S. Environmental Protection Agency (EPA), Biopesticides are certain types of pesticides derived from such natural materials as animals, plants, bacteria, and certain minerals. Biopesticides fall into three major classes: (1) Microbial pesticides consist of a microorganism (e.g., a bacterium, fungus, virus or protozoan) as the active ingredient. The most widely used microbial pesticides are subspecies and strains of Bacillus thuringiensis, or Bt. Each strain of this bacterium produces a different mix of proteins, and specifically kills one or a few related species of insect larvae. (2) Plant-Incorporated-Protectants (PIPs) are pesticidal substances that plants produce from genetic material that has been added to the plant. (3) Biochemical pesticides are naturally occurring substances that control pests by non-toxic mechanisms. Conventional pesticides, by contrast, are generally synthetic materials that directly kill or inactivate the pest. Biochemical pesticides include substances, such as insect sex pheromones, that interfere with mating, as well as various scented plant extracts that attract insect pests to traps.

Exemplary compounds mostly used as larvicides include, but are not limited to, organophosphates and surface oils and films.

Further examples of larvicides include, but are not limited to, waste oil or diesel oil products. Paris green dust is an arsenical insecticide, used along with undiluted diesel oil, and dichloro-diphenyl-trichloroethane (DDT), used as both an adulticide and a larvicide, malathion, an organophosphate (OP) compound, increased, but resistance was soon observed. The term organophosphate (OP) refers to all pesticides containing phosphorus, acting through inhibition of the activity of cholinesterase enzymes at the neuromuscular junction. Temephos is currently the only OP registered for use as a larvicide in the US.

Biolarvicides are comprised of two major categories: (1) Microbial agents (e.g., bacteria) and (2) Biochemical agents (e.g., pheromones, hormones, growth regulators, and enzymes). Regarding microbial agents, controlled-release formulations of at least one biological pesticidal ingredient are disclosed in U.S. Pat. No. 4,865,842; control of mosquito larvae with a spore-forming Bacillus ONR-60A is disclosed in U.S. Pat. No. 4,166,112; novel Bacillus thuringiensis isolates with activity against dipteran insect pests are disclosed in U.S. Pat. Nos. 5,275,815 and 5,847,079; a biologically pure culture of a Bacillus thuringiensis strain with activity against insect pests of the order Diptera is disclosed in U.S. Pat. No. 5,912,162; a recombinantly derived biopesticide active against Diptera including cyanobacteria transformed with a plasmid containing a B. thuringiensis subsp. israelensis dipteracidal protein translationally fused to a strong, highly active native cyanobacteria's regulatory gene sequence is disclosed in U.S. Pat. No. 5,518,897 and a formulation of Bacillus thuringiensis subspecies Israelensis and Bacillus sphaericus to manage mosquito larvicide resistance U.S. Pat. No. 7,989,180 B2.

Biochemical agents such as Insect Growth Regulators (IGRS) mimics naturally occurring insect biochemicals and Methoprene (a juvenile hormone (JH) analog) is a commercially available insecticide of this class.

According to one embodiment, the larvicide is selected from the group consisting of Temephos, Diflubenzuron, Methoprene, or a microbial larvicide such as Bacillus sphaericus or Bacillus thuringiensis israelensis.

According to one embodiment, the larvicide comprises an adulticide.

Exemplary adulticides include, but are not limited to, deltamethrin, malathion, naled, chlorpyrifos, permethrin, resmethrin or sumithrin.

According to a specific embodiment, the cells are formulated by any means known in the art. The methods for preparing such formulations include, e.g., desiccation, lyophilization, homogenization, extraction, filtration, encapsulation centrifugation, sedimentation, or concentration of one or more cell types.

In one embodiment, the composition comprises an oil flowable suspension. For example, in some embodiments, oil flowable or aqueous solutions may be formulated to contain lysed or unlysed cells, spores, or crystals.

In a further embodiment, the composition may be formulated as a water dispersible granule or powder.

In yet a further embodiment, the compositions of the present invention may also comprise a wettable powder, spray, emulsion, colloid, aqueous or organic solution, dust, pellet, or colloidal concentrate. Dry forms of the compositions may be formulated to dissolve immediately upon wetting, or alternatively, dissolve in a controlled-release, sustained-release, or other time-dependent manner.

Alternatively or additionally, the composition may comprise an aqueous solution. Such aqueous solutions or suspensions may be provided as a concentrated stock solution which is diluted prior to application, or alternatively, as a diluted solution ready-to-apply. Such compositions may be formulated in a variety of ways. They may be employed as wettable powders, granules or dusts, by mixing with various inert materials, such as inorganic minerals (silicone or silicon derivatives, phyllosilicates, carbonates, sulfates, phosphates, and the like) or botanical materials (powdered corncobs, rice hulls, walnut shells, and the like).

The formulations may include spreader-sticker adjuvants, stabilizing agents, other pesticidal additives, or surfactants. Liquid formulations may be employed as foams, suspensions, emulsifiable concentrates, or the like. The ingredients may include Theological agents, surfactants, emulsifiers, dispersants, or polymers.

As mentioned, the dsRNA of the invention may be administered as a naked dsRNA. Alternatively, the dsRNA of the invention may be conjugated to a carrier known to one of skill in the art, such as a transfection agent e.g. PEI or chitosan or a protein/lipid carrier.

The compositions may be formulated prior to administration in an appropriate means such as lyophilized, freeze-dried, microencapsulated, desiccated, or in an aqueous carrier, medium or suitable diluent, such as saline or other buffer. Suitable agricultural carriers can be solid, semi-solid or liquid and are well known in the art. The term “agriculturally-acceptable carrier” covers all adjuvants, e.g., inert components, dispersants, surfactants, tackifiers, binders, etc. that are ordinarily used in pesticide formulation technology.

According to one embodiment, the composition is formulated as a semi-solid such as in agarose (e.g. agarose cubes).

The mosquito larva food containing dsRNA may be prepared by any method known to one of skill in the art. Thus, for example, cubes of dsRNA-containing mosquito food may be prepared by first mixing 10-500 μg, e.g. 300 μg of dsRNA with 3 to 300 μg, e.g. 10 μg of a transfection agent e.g. Polyethylenimine 25 kDa linear (Polysciences) in 10-500 μL, e.g. 200 μL of sterile water. Alternatively, 2 different dsRNA (10-500 μg, e.g. 150 μg of each) plus 3 to 300 μg, e.g. 30 μg of Polyethylenimine may be mixed in 10-500 μL, e.g. 200 μL of sterile water. Alternatively, cubes of dsRNA-containing mosquito food may be prepared without the addition of transfection reagents. Then, a suspension of ground mosquito larval food (1-20 grams/100 mL e.g. 6 grams/100 mL) may be prepared with 2% agarose (Fisher Scientific). The food/agarose mixture can then be heated to 53-57° C., e.g. 55° C., and 10-500 μL, e.g. 200 μL of the mixture can then be transferred to the tubes containing 10-500 μL, e.g. 200 μL of dsRNA+PEI or dsRNA only. The mixture is then allowed to solidify into a gel. The solidified gel containing both the food and dsRNA can be cut into small pieces (approximately 1-10 mm, e.g. 1 mm, thick) using a razor blade, and can be used to feed mosquito larvae in water.

Compositions of the invention can be used to control or exterminate mosquitoes. Such an application comprises feeding larvae of the mosquitoes with an effective amount of the composition to thereby control or exterminate the mosquitoes.

According to a specific embodiment, the composition may be applied to standing water.

The pesticidal compositions of the invention may be employed in the method of the invention singly or in combination with other compounds, including, but not limited to, other pesticides (not included in the formulation as described above).

Regardless of the method of application, the amount of the active component(s) are applied at a larvicidally-effective amount, which will vary depending on factors such as, for example, the specific mosquito to be controlled, the water source to be treated, the environmental conditions, and the method, rate, and quantity of application of the larvicidally-active composition.

The concentration of larvicidal composition that is used for environmental, systemic, or foliar application will vary widely depending upon the nature of the particular formulation, means of application, environmental conditions, and degree of biocidal activity.

The larvae may be pathogenically infected as described above or uninfected larvae.

The concentration of the composition that is used for environmental, systemic, or foliar application will vary widely depending upon the nature of the particular formulation, means of application, environmental conditions, and degree of activity.

Exemplary concentrations of dsRNA in the composition include, but are not limited to, about 1 pg-10 μg of dsRNA/μl, about 1 pg-1 μg of dsRNA/μl, about 1 pg-0.1 μg of dsRNA/μl, about 1 pg-0.01 μg of dsRNA/μl, about 1 pg-0.001 μg of dsRNA/μl, about 0.001 μg-10 μg of dsRNA/μl, about 0.001 μg-5 μg of dsRNA/μl, about 0.001 μg-1 μg of dsRNA/μl, about 0.001 μg-0.1 μg of dsRNA/μl, about 0.001 μg-0.01 μg of dsRNA/μl, about 0.01 μg-10 μg of dsRNA/μl, about 0.01 μg-5 μg of dsRNA/μl, about 0.01 μg-1 μg of dsRNA/μl, about 0.01 μg-0.1 μg of dsRNA/μl, about 0.1 μg-10 μg of dsRNA/μl, about 0.1 μg-5 μg of dsRNA/μl, about 0.5 μg-5 μg of dsRNA/μl, about 0.5 μg-10 μg of dsRNA/μl, about 1 μg-5 μg of dsRNA/μl, or about 1 μg-10 μg of dsRNA/μl.

When formulated as a feed, the dsRNA may be effected at a dose of 1 pg/larvae-1000 μg/larvae, 1 pg/larvae-500 μg/larvae, 1 pg/larvae-100 μg/larvae, 1 pg/larvae-10 μg/larvae, 1 pg/larvae-1 μg/larvae, 1 pg/larvae-0.1 μg/larvae, 1 pg/larvae-0.01 μg/larvae, 1 pg/larvae-0.001 μg/larvae, 0.001-1000 μg/larvae, 0.001-500 μg/larvae, 0.001-100 μg/larvae, 0.001-50 μg/larvae, 0.001-10 μg/larvae, 0.001-1 μg/larvae, 0.001-0.1 μg/larvae, 0.001-0.01 μg/larvae, 0.01-1000 μg/larvae, 0.01-500 μg/larvae, 0.01-100 μg/larvae, 0.01-50 μg/larvae, 0.01-10 μg/larvae, 0.01-1 μg/larvae, 0.01-0.1 μg/larvae, 0.1-1000 μg/larvae, 0.1-500 μg/larvae, 0.1-100 μg/larvae, 0.1-50 μg/larvae, 0.1-10 μg/larvae, 0.1-1 μg/larvae, 1-1000 μg/larvae, 1-500 μg/larvae, 1-100 μg/larvae, 1-50 μg/larvae, 1-10 μg/larvae, 10-1000 μg/larvae, 10-500 μg/larvae, 10-100 μg/larvae, 10-50 μg/larvae, 50-1000 μg/larvae, 50-500 μg/larvae, 50-400 μg/larvae, 50-300 μg/larvae, 100-500 μg/larvae, 100-300 μg/larvae, 200-500 μg/larvae, 200-300 μg/larvae, or 300-500 μg/larvae.

According to some embodiments, the nucleic acid agent is provided in amounts effective to reduce or suppress expression of at least one mosquito gene product. As used herein “a suppressive amount” or “an effective amount” refers to an amount of dsRNA which is sufficient to downregulate (reduce expression of) the target gene by at least 20%, 30%, 40%, 50%, or more, say 60%, 70%, 80%, 90% or more even 100%.

Testing the efficacy of gene silencing can be effected using any method known in the art. For example, using quantitative RT-PCR measuring gene knockdown. Thus, for example, ten to twenty larvae or mosquitoes from each treatment group can be collected and pooled together. RNA can be extracted therefrom and cDNA syntheses can be performed. The cDNA can then be used to assess the extent of RNAi by measuring levels of gene expression using qRT-PCR.

Compositions of the present invention can be packed in a kit including the cells which comprise the nucleic acid larvicides, instructions for administration of the composition to mosquito larvae.

Compositions of some embodiments of the invention may, if desired, be presented in a pack or dispenser device, which may contain one or more dosage forms containing the active ingredient. The pack may, for example, comprise metal or plastic foil, such as a blister pack. The pack or dispenser device may be accompanied by instructions for administration to the mosquito larvae.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Materials and Experimental Procedures

Mosquito Maintenance

Mosquitoes were taken from an Ae. aegypti colony of the Rockefeller strain or from a mosquito field population of Ae. aegypti isolated from urban area of Rio de Janeiro, Brazil. Both lineages were reared continuously in the laboratory at 28° C. and 70-80% relative humidity. Adult mosquitoes were maintained in a 10% sucrose solution, and the adult females were fed with sheep blood for egg laying. The larvae were reared on dog/cat food unless stated otherwise.

Introducing dsRNA into a Mosquito Larvae

Three different approaches were evaluated for treatment with dsRNA:

A) Soaking with “Naked” dsRNA

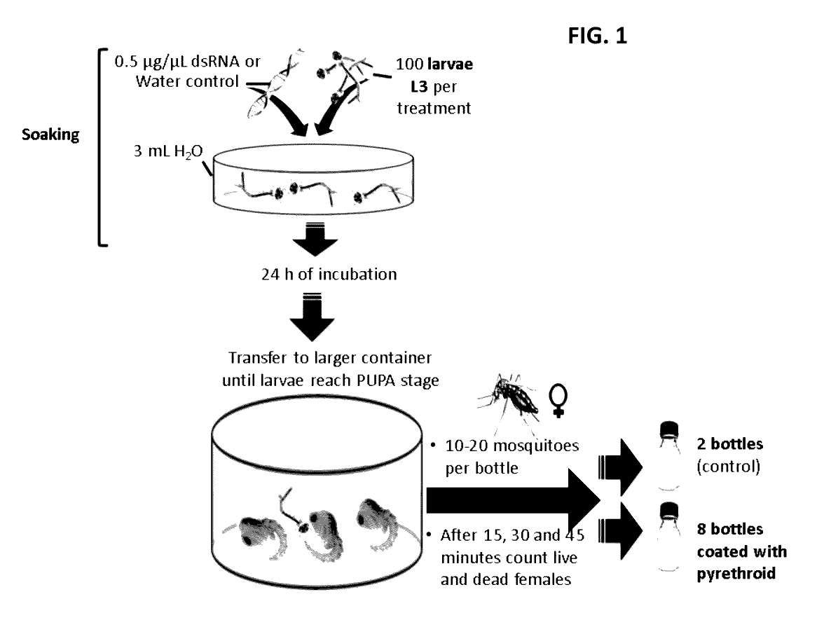

Third instar larvae were treated (in groups of 100 larvae) in a final volume of 3 mL of dsRNA solution in autoclaved water (0.5 μg/μL for sodium channel (AAEL008297), PgP (AAEL010379) and Ago3 (AAEL007823) dsRNA, or 0.1 μg/μL for CYP9J26 (JF924909.1). The control group was kept in 3 ml sterile water only. Larvae were soaked in the dsRNA solutions for 24 hr at 27° C., and then transferred into new containers (300 larvae/1500 mL of chlorine-free tap water), which were also maintained at 27° C., and were provided 6 mg/100 mL lab dog/cat diet (Purina Mills) suspended in water as a source of food on a daily basis. As pupae developed, they were transferred to individual vials to await eclosion and sex sorting. For bioassays purpose only females up to five days old were used. See Flowchart 1, FIG. 1 for detailed explanation of this experiment.

B) Soaking with “Naked” dsRNA Plus Additional Larvae Feeding with Food-Containing dsRNA

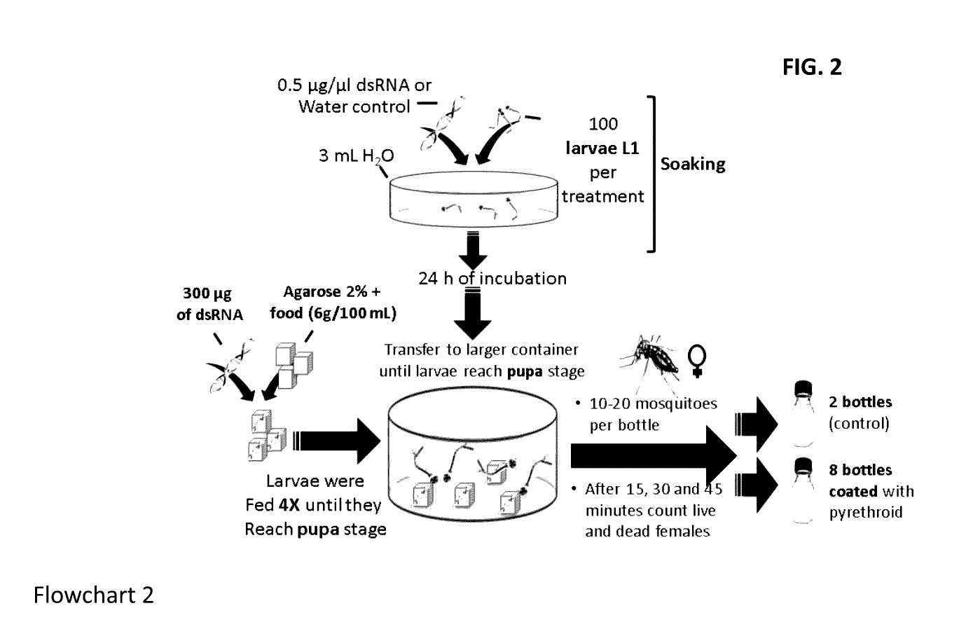

After soaking in the dsRNA solutions for 24 hr at 27° C., the larvae were transferred into new containers (300 larvae/1500 mL of chlorine-free tap water), and were provided agarose cubes containing 300 μg of dsRNA once a day for a total of four days. The larvae were reared until adult stage. For bioassays purpose only females up to five days old are used. See Flowchart 2, FIG. 2 for detailed explanation of this experiment.

C) Larvae Feeding with Food-Containing dsRNA Only

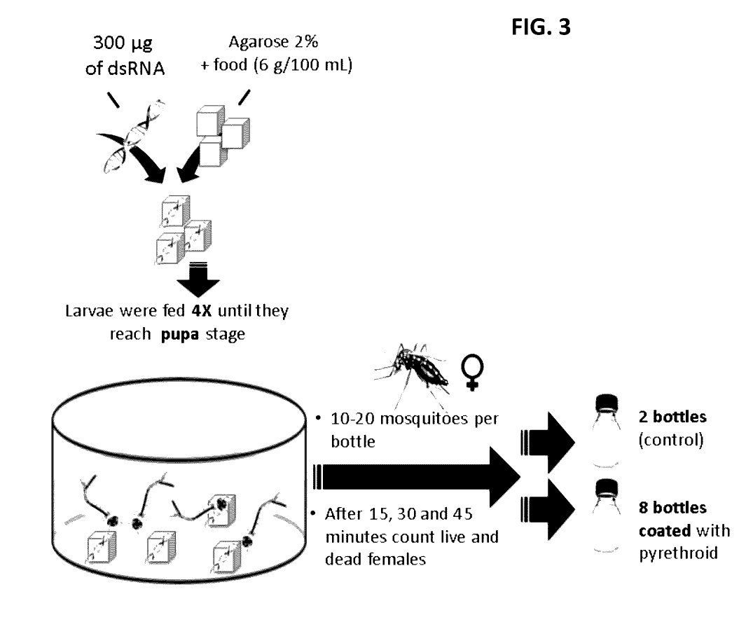

Third instar larvae were fed (in groups of 300 larvae) in a final volume of 1500 mL of chlorine-free tap water with agarose cubes containing 300 μg of dsRNA once a day for a total of four days. The larvae were reared until adult stage. For bioassays purpose only females up to five days old are used. See Flowchart 3, FIG. 3 for detailed explanation of this experiment.

Bioassay with Pyrethroid

CDC Bottle Bioassays—

Bottles were prepared following the Brogdon and McAllister (1998) protocol [Brogdon and McAllister (1998) Emerg Infect Dis 4:605-613]. Fifteen-twenty non-blood-fed females from each site were introduced in 250 mL glass bottles impregnated with different concentrations of deltamethrin (Sigma-Aldrich) in 1 ml acetone. Each test consisted of four impregnated bottles and one control bottle. The control bottle contained acetone with no insecticide. At least three tests were conducted for each insecticide and population. Immediately prior to use, all insecticide solutions were prepared fresh from stock solutions. At 15, 30 and 45 min intervals, the number of live and dead mosquitoes in each bottle was recorded. The mortality criteria included mosquitoes with difficulties flying or standing on the bottle's surface. Mosquitoes that survived the appropriate dose for insecticide were considered to be resistant [Brogdon and McAllister (1998), supra].

Preparation of Mosquito Larval Food Containing dsRNA

Cubes of dsRNA-containing mosquito food were prepared as follows: First, 300 μg of dsRNA were mixed with 30 μg of Polyethylenimine 25 kD linear (Polysciences) in 200 μL of sterile water. Then, a suspension of ground mosquito larval food (6 grams/100 mL) was prepared with 2% agarose (Fisher Scientific). The food/agarose mixture was heated to 55° C. and 200 μL of the mixture was then transferred to the tubes containing 200 μL of dsRNA+PEI or water only (control). The mixture was then allowed to solidify into a gel. The solidified gel containing both the food and dsRNA was cut into small pieces (approximately 1 mm thick) using a razor blade, which were then used to feed mosquito larvae in water.

RNA Isolation and dsRNA Production

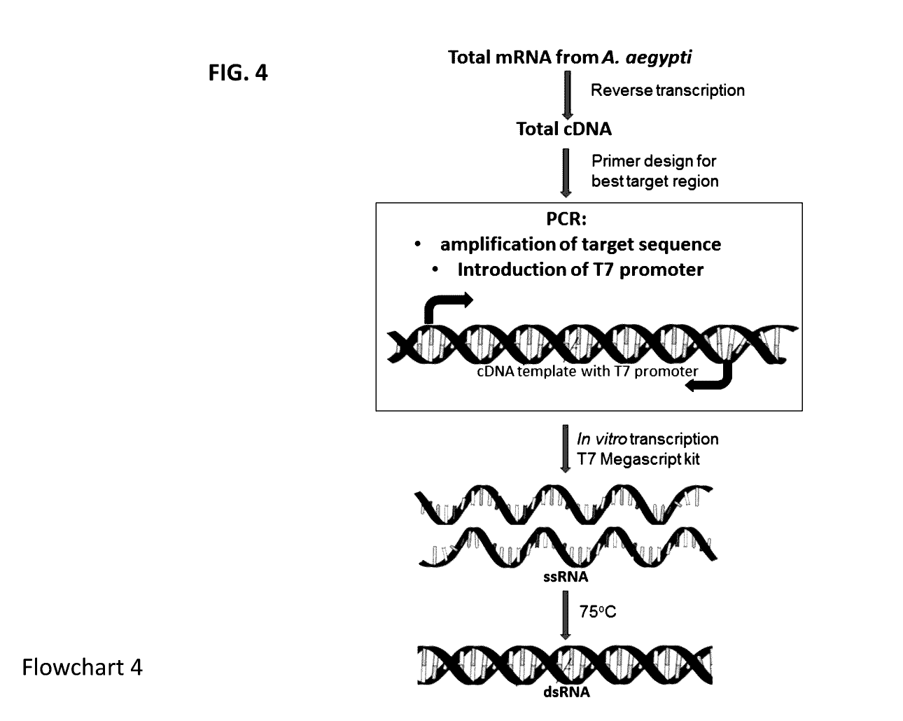

Total RNA was extracted from groups of five Ae. aegypti fourth instar larvae and early adult male/female Ae. aegypti, using TRIzol (Invitrogen, Carlsbad, Calif., USA) according to the manufacturer's instructions. RNA was treated with amplification grade DNase I (Invitrogen) and 1 μg was used to synthesize cDNA using a First Strand cDNA Synthesis kit (Invitrogen). The cDNA served as template DNA for PCR amplification of gene fragments using the primers listed in Table 6, below. PCR products were purified using a QIAquick PCR purification kit (Qiagen). The MEGAscript RNAi kit (Ambion) was then used for in vitro transcription and purification of dsRNAs. See Flowchart 4, FIG. 4 for detailed explanation production off dsRNA.

TABLE 6
qPCR primers and dsRNA
sequences for adulticide targets

| | Accession | | |
|---|---|---|---|
| Target gene | number | dsRNA sequence | qPCR primers (5′-3′) |
| P-glycoprotein | XM_001654442.1 | SEQ ID NO: 1822 | F: GCGCGCTCGTTCAGTATTTA |
| (AAEL010379) | | | (SEQ ID NO: 1814) |
| | | | R: ACACCCGTTACGGCACAATA |
| | | | (SEQ ID NO: 1815) |
| Argonaute-3 | XM_001652895.1 | SEQ ID NO: 1823 | F: TCGGCATTCGTAGCTTCGTT |
| (AAEL007823) | | | (SEQ ID NO: 1816) |
| | | | R: GCAGCTGACAGTTTGCCTTC |
| | | | (SEQ ID NO: 1817) |
| Cytochrome p450 | JF924909.1 | SEQ ID NO: 1824 | F: CCGTTTGGTATCGGCCCAAG |
| (CYP9J26) | XM_001649047.2 | | (SEQ ID NO: 1818) |
| JF924909.1 | | | R: GTCTTTGCGCCTCGGACG |
| | | | (SEQ ID NO: 1819) |
| Sodium channel | KC107440.1 | SEQ ID NO: 1825 | F: CTGGAGTCGGTGAGCGAAA |
| (AAEL008297) | XM_001653136.1 | | (SEQ ID NO: 1820) |
| | | | R: TACGTATCGTAAACGCGCTC |
| | | | (SEQ ID NO: 1821) |

qPCR Analysis

Approximately 1000 ng first-strand cDNA obtained as described previously was used as template. The qPCR reactions were performed using SYBR® Green PCR Master Mix (Applied Biosystems) following the manufacturer's instructions. Briefly, approximately 50 cDNA and gene-specific primers (600 nM) were used for each reaction mixture. qPCR conditions used were 10 min at 95° C. followed by 35 cycles of 15 s at 94° C., 15 s at 54° C. and 60 s at 72° C. The ribosomal protein S7 and tubulin were used as the reference gene to normalize expression levels amongst the samples. Raw quantification cycle (Cq) values normalized against those of the tubulin and S7 standards were then used to calculate the relative expression levels in samples using the 2^{-ΔΔct} method [Livak & Schmittgen, (2001) Methods. 25(4):402-8.). Results (mean±SD) are representative of at least two independent experiments performed in triplicate.

### Results

### Characterization of Insecticide Resistance Using Two Different Strains of Aedes aegypti Mosquitoes

Vector control strategies employed for Aedes control are mainly anti-larval measures, source reduction and use of adulticides (pyrethroids). Pyrethroids are a major class of insecticides, which show low mammalian toxicity and fast knockdown activity. Unfortunately, the intensive use of pyrethroids, including their indirect use in agriculture, has led to reports of reduced efficacy. One of the mechanisms of resistance in insects against pyrethroids is knockdown resistance (kdr) which is conferred by mutation(s) in the target site, the voltage gated sodium channel (VGSC). Several kdr mutations have been reported in many insects of agricultural and medical importance including Ae. aegypti. In Ae. Aegypti, eleven non-synonymous mutations at nine different loci have been reported [Med Vet Ent 17: 87-94.; Insect Mol Biol 16: 785-798.; Insect Biochem Mol Biol 39: 272-278.], amongst which mutations at three loci, i.e., Iso1011 (IRM/V) and Va11016 (VRG/I) in domain II and F1534 (FRC) in domain III are most commonly reported as contributing to pyrethroid resistance.

Using a population of mosquitoes that shows increased pyrethroid resistance, the present inventors target (during larval stage) several genes associated with resistance to pyrethroid in order to break resistance to insecticide at the adult stage.

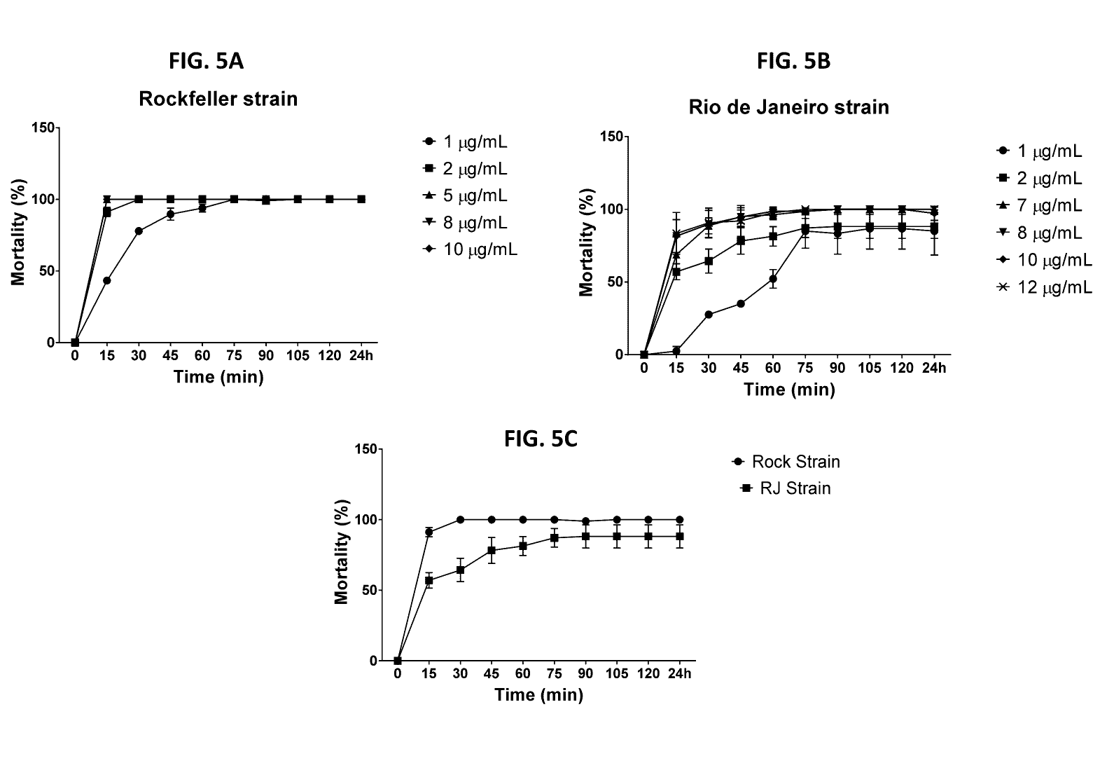

A diagnostic dosage (DD) was established for the insecticide using the Rockefeller reference susceptible Ae. aegypti strain and a resistance threshold (RT), time in which 98-100% mortality was observed in the Rockefeller strain, was then calculated. Using the DD (2 μg/mL of deltamethrin) (FIGS. 5A-C) is was possible to demonstrate that this dose killed only 63.95% of the Rio de Janeiro strain whereas 100% of the mosquitoes from the Rockefeller strain were dead. Therefore, it was concluded that 36.05% of the mosquitoes in this population (RJ) are resistant to deltamethrin.

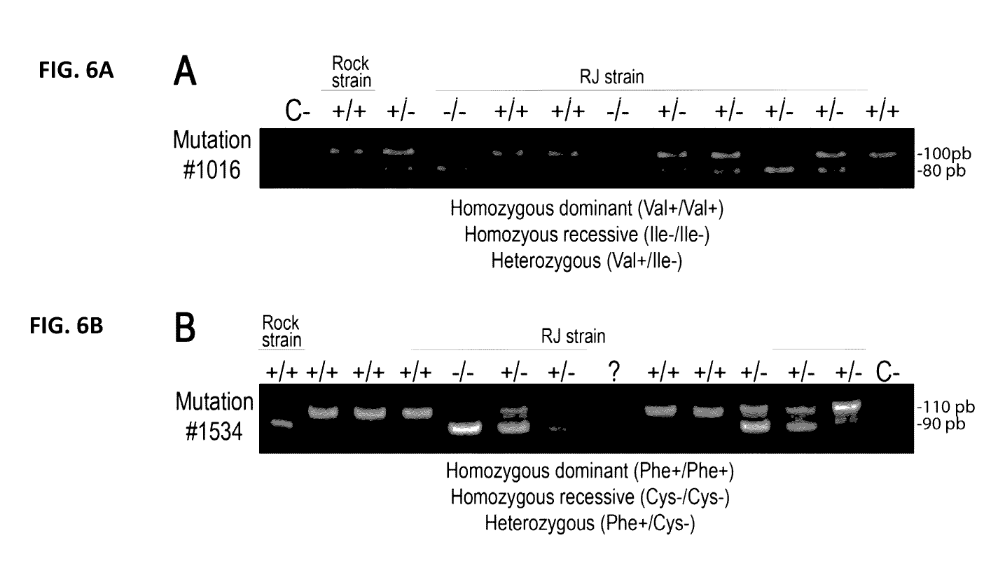

To further confirm the resistance status of the Rio de Janeiro strain, the kdr mutations reported as contributing to pyrethroid resistance were assessed. In FIGS. 6A-B, the present inventors show that V1016G and F1534C were both detected in the RJ strain. Indeed, the V1016G and F1534C mutation were detected in 49% and 60% of the mosquitoes from Rio de Janeiro strain, respectively.

### Silencing of Sodium Channel During Larval Development Increases the Susceptibility of Adult Mosquitoes to Pyrethroid

Using the first approach (soaking with “naked” dsRNA), mosquito larvae (RJ strain) were treated with three different dsRNA: Ago3, P-glycoprotein and Sodium channel. Treatment with dsRNA against sodium channel increased substantially the susceptibility of mosquitoes to the insecticide (FIG. 7A). Interestingly, female mosquitoes showed a decreased expression in the mRNA level for sodium channel before deltamethrin treatment (FIG. 7B). When compared to water-treated mosquitoes only, dead female mosquitoes previously treated with dsRNA showed a striking decrease in mRNA expression level for sodium channel (FIG. 7C).

In order to test the second approach (soaking with “naked” dsRNA plus additional larvae feeding with food-containing dsRNA), mosquito larvae (L3) were first soaked with dsRNA (sodium channel, 0.5 μg/μL) for 24 hours. Then, larvae were treated 4 times with food-containing dsRNA and reared until adult stage. Although there was no obvious advantage in using this approach when compared to soaking with naked dsRNA alone, treatment with dsRNA against sodium channel increased the susceptibility of mosquitoes to deltamethrin (FIG. 8).

This approach was also tested using dsRNA to target Cytochrome p450 (CYP9J26). As can be seen in the FIG. 9, dsRNA-treated mosquitoes were more sensitive to deltamethrin during the first 15 minutes of contact with deltamethrin.

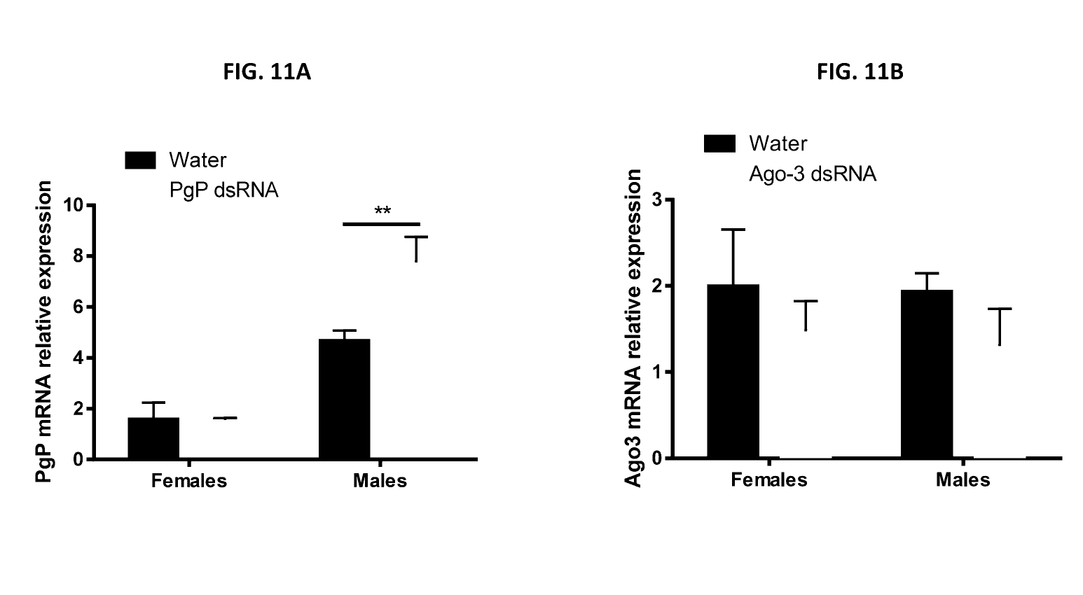

It is important to note that that 24 and 48 hours after the end of dsRNA treatment, decreased mRNA levels were detected in mosquito adults that were treated with PgP, Ago3 or sodium channel dsRNA as larvae (FIGS. 10A-C). However, PgP and Ago3 mRNA expression reached normal levels when mosquitoes became adults (FIG. 11A-B, respectively).

### Example 2

### Materials and Experimental Procedures

### Mosquito Maintenance

Mosquitoes were taken from an Ae. aegypti colony of the Rockefeller strain, which were reared continuously in the laboratory at 28° C. and 70-80% relative humidity. Adult mosquitoes were maintained in a 10% sucrose solution, and the adult females were fed with sheep blood for egg laying. The larvae were reared on dog/cat food unless stated otherwise.

Introducing dsRNA into a Mosquito Larvae

Soaking with “Naked” dsRNA Plus Additional Larvae Feeding with Food-Containing dsRNA

Third instar larvae were treated (in groups of 100 larvae) in a final volume of 3 mL of dsRNA solution in autoclaved water (dsRNA concentrations are shown in Table 7, below). The control group was kept in 3 ml sterile water only. After soaking in the dsRNA solutions for 24 hr at 27° C., the larvae were transferred into new containers (300 larvae/1500 mL of chlorine-free tap water) and provided 6 mg/100 mL lab dog/cat diet (Purina Mills) suspended in water and agarose cubes containing 300 μg of dsRNA once a day for a total of two days. As pupae developed, they were transferred to individual vials to await eclosion and sex sorting. For bioassays purpose only females up to five days old were used.

The pupae mortality was calculated based on the initial number of treated larvae (300) (Mortality of pupae=Total number of pupae/300). Once the adults emerged they start to copulate.

TABLE 7
dsRNA concentrations

| dsRNA | Concentration (μg/μL each 100 larvae) |
|---|---|
| Ago3 (AAEL007823) | 0.5 |
| Aub (AAEL007698) | 0.5 |
| AF510492.1 | 0.1 |
| AY531222.2 | 0.02 |
| AAEL017015 | 0.06 |
| AAEL005212 | 0.06 |
| AAEL005922 | 0.05 |
| AAEL000903 | 0.06 |

Preparation of Mosquito Larval Food Containing dsRNA

Cubes of dsRNA-containing mosquito food were prepared as follows: First, 300 μg of dsRNA were mixed with 30 μg of Polyethylenimine 25 kD linear (Polysciences) in 200 μL of sterile water. Then, a suspension of ground mosquito larval food (6 grams/100 mL) was prepared with 2% agarose (Fisher Scientific). The food/agarose mixture was heated to 55° C. and 200 μL of the mixture was then transferred to the tubes containing 200 μL of dsRNA+PEI or water only (control). The mixture was then allowed to solidify into a gel. The solidified gel containing both the food and dsRNA was cut into small pieces (approximately 1 mm thick) using a razor blade, which were then used to feed mosquito larvae in water.

Blood Feeding

Five to seven days following adult emergence, dsRNA-treated or untreated control mosquitoes received defibrinated sheep blood through a membrane feeder. Thirty minutes after receiving a blood meal, three groups of 15 engorged females were separated inside a new cartoon cage to perform the oviposition assay.

Oviposition Assay and Hatching Rate

Five days after the blood meal, an ovipositon cup was place inside each cage containing 15 females to allow the females to lay their eggs. The oviposition cup was changed every 24 hours for 3 consecutive days. The number of eggs laid was counted and used to check the viability and egg hatching rate.

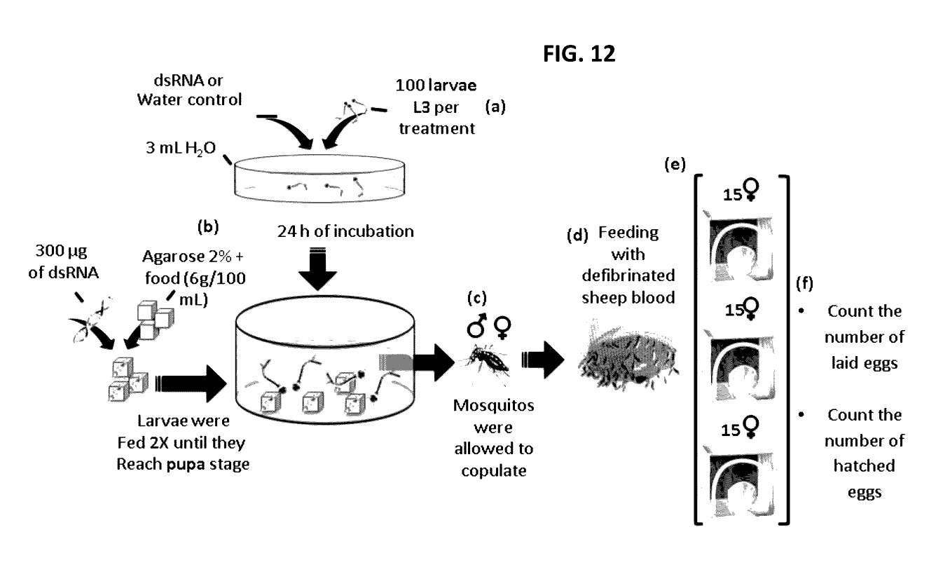

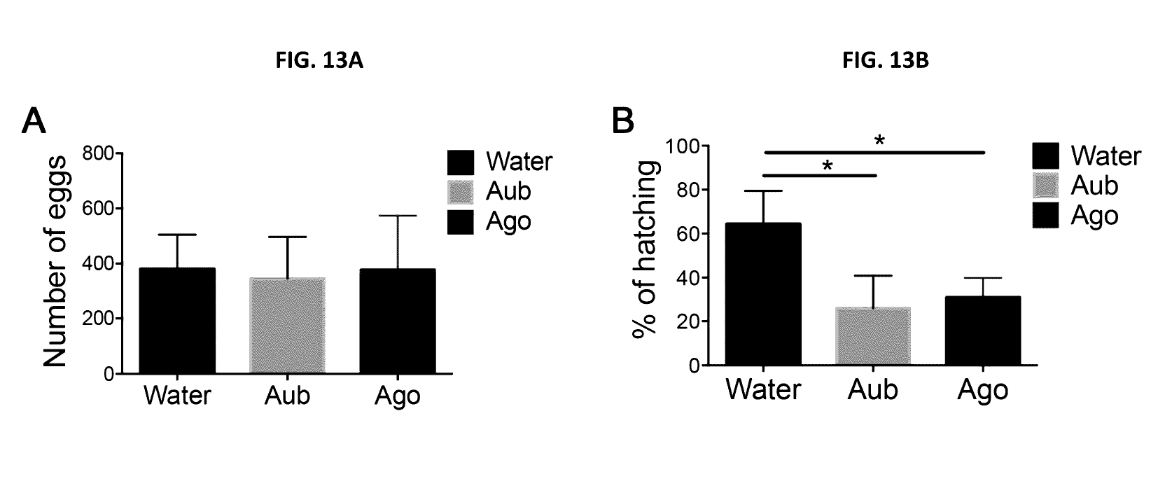

To check the viability of the eggs the oviposition paper were kept to dry and embrionate for a period minimum of 5 days. After this time the ovipositions papers containing the eggs were placed inside a tray with aged water and food and wait for the eggs to hatch for a period of 24 hours. The hatching rate (HR) for each treatment were calculated as follow: HR=total number of hatched larvae/total number of eggs oviposited). FIG. 12 describes the experiment.

RNA Isolation and dsRNA Production

Total RNA was extracted from groups of five Ae. aegypti fourth instar larvae and early adult male/female Ae. aegypti, using TRIzol (Invitrogen, Carlsbad, Calif., USA) according to the manufacturer's instructions. RNA was treated with amplification grade DNase I (Invitrogen) and 1 μg was used to synthesize cDNA using a First Strand cDNA Synthesis kit (Invitrogen). The cDNA served as template DNA for PCR amplification of gene fragments using the primers listed in Table 8, below. PCR products were purified using a QIAquick PCR purification kit (Qiagen). The MEGAscript RNAi kit (Ambion) was then used for in vitro transcription and purification of dsRNAs sequences (Table 9, below).

TABLE 8
qPCR primers for fertility targets

| | Accession | qPCR primers |
|---|---|---|
| Target gene | number | (5′-3′) |
| Argonaute-3 | XM_001652895.1 | F: TCGGCATTCGTAGC |
| AAEL007823 | | TTCGTT |
| | | (SEQ ID NO: 1833) |
| | | R: GCAGCTGACAGTTT |
| | | GCCTTC |
| | | (SEQ ID NO: 1834) |
| AuB | | F: CAGAATCCCAGACC |
| AAEL007698 | | CGGAAC |
| | | (SEQ ID NO: 1835) |
| | | R: TTGGCGAAACCGTA |
| | | CCTTGA |
| | | (SEQ ID NO: 1836) |
| AeSCP-2 | AF510492 | F: TAAGCGTCTGGAGA |
| (AF510492.1) | | GCATCG |
| | | (SEQ ID NO: 1837) |
| | | R: CTCGACCAGCTGAC |
| | | GTTCTT |
| | | (SEQ ID NO: 1838) |
| AeAct-4 | AY531222 | F: GTTTCGCTGGTGAT |
| (AY531222.2) | | GATGCC |
| | | (SEQ ID NO: 1839) |
| | | R: GGTGAGGATACCTC |
| | | GCTTCG |
| | | (SEQ ID NO: 1840) |
| AAEL002000 | XM_001660689.1 | F: CGTCAAGGTGGAAG |
| | | ATTTCGG |
| | | (SEQ ID NO: 1841) |
| | | R: CGGCATCCGGATTA |
| | | TTGTCG |
| | | (SEQ ID NO: 1842) |
| AAEL005747 | XM_001651331.1 | F: TGCTGTCCACCAGT |
| | | ATGAGC |
| | | (SEQ ID NO: 1843) |
| | | R: TCCTCCGATGGCAT |
| | | TGCTTT |
| | | (SEQ ID NO: 1844) |
| AAEL005656 | XM_001651169.1 | F: GCGCATGAAGAAGA |
| | | AGCTGG |
| | | (SEQ ID NO: 1845) |
| | | R: TTTGTGCCTCTGCA |
| | | TTTGCC |
| | | (SEQ ID NO: 1846) |
| AAEL017015 | XM_011494635.1 | F: GCCTACCAAGCTCC |
| | | GCAAAT |
| | | (SEQ ID NO: 1847) |
| | | R: GACGATGTCCTGCT |
| | | GTTCGT |
| | | (SEQ ID NO: 1848) |
| AAEL005212 | XM_001650421.1 | F: TGTGGACGCTAAGG |
| | | AACAGCC |
| | | (SEQ ID NO: 1849) |
| | | R: CATCGAGCCCCAAG |
| | | CATCC |
| | | (SEQ ID NO: 1850) |
| AAEL005922 | XM_001651632.1 | F: GAAGATCAATGCAC |
| | | CACCGC |
| | | (SEQ ID NO: 1851) |
| | | R: GGACGCGATCTACG |
| | | AGGTTT |
| | | (SEQ ID NO: 1852) |
| AAEL000903 | XM_001651555.1 | F: TACCGGACACCGTC |
| | | AAGAAG |
| | | (SEQ ID NO: 1853) |
| | | R: CTAAATATCGATAC |
| | | CCTCCTGCTG |
| | | (SEQ ID NO: 1854) |
| AAEL005049 | XM_001650243.1 | F: ACTCGGAAGCAGTG |
| | | GTAACG |
| | | (SEQ ID NO: 1855) |
| | | R: ATCTGCATTCCTTC |
| | | CGGCTT |
| | | (SEQ ID NO: 1856) |

TABLE 9
dsRNA sequences for fertility targets

| Target gene | Accession number | dsRNA sequence |
|---|---|---|
| Argonaute-3 | XM_001652895.1 | SEQ ID NO: 1857 |
| AAEL007823 | | |
| AuB | | SEQ ID NO: 1858 |
| AAEL007698 | | |
| AeSCP-2 | AF510492 | SEQ ID NO: 1859 |
| (AF510492.1) | | |
| AeAct-4 | AY531222 | SEQ ID NO: 1860 |
| (AY531222.2) | | |
| AAEL002000 | XM_001660689.1 | SEQ ID NO: 1861 |
| AAEL005747 | XM_001651331.1 | SEQ ID NO: 1862 |
| AAEL005656 | XM_001651169.1 | SEQ ID NO: 1863 |
| AAEL017015 | XM_011494635.1 | SEQ ID NO: 1864 |
| AAEL005212 | XM_001650421.1 | SEQ ID NO: 1865 |
| AAEL005922 | XM_001651632.1 | SEQ ID NO: 1866 |
| AAEL000903 | XM_001651555.1 | SEQ ID NO: 1867 |
| AAEL005049 | XM_001650243.1 | SEQ ID NO: 1868 |

qPCR Analysis

Approximately 1000 ng first-strand cDNA obtained as described previously was used as template. The qPCR reactions were performed using SYBR® Green PCR Master Mix (Applied Biosystems) following the manufacturer's instructions. Briefly, approximately 50 cDNA and gene-specific primers (600 nM) were used for each reaction mixture. qPCR conditions used were 10 min at 95° C. followed by 35 cycles of 15 s at 94° C., 15 s at 54° C. and 60 s at 72° C. The ribosomal protein S7 and tubulin were used as the reference gene to normalize expression levels amongst the samples. Raw quantification cycle (Cq) values normalized against those of the tubulin and S7 standards were then used to calculate the relative expression levels in samples using the 2^{-ΔΔct} method [Livak & Schmittgen, (2001) Methods. 25(4):402-8]. Results (mean±SD) are representative of at least two independent experiments performed in triplicate.

### Results

Gene Silencing with dsRNA During Larval Development Decreases the Number of Hatchings

The sterile insect technique (SIT) is a non-insecticidal control method that relies on the release of sterile male mosquitoes that search for and mate with wild females, preventing offspring. This approach has been used successfully to control various insect pest species. Recently, a dsRNA-based method to produce sterile male mosquitoes was described [Whyard et al., Parasit Vectors. (2015) 8: 96].

The present inventors hypothesized that dsRNA could be used to produce effective sterile male/female Ae. aegypti mosquitoes by targeting genes expressed mainly (but not exclusively) in male testes and/or female ovary. Since sterile female insects can still damage crops and transmit disease, ideally the product will include dsRNA sequences to induce mortality in infected-mosquitoes or reduce resistance to pyrethroids.

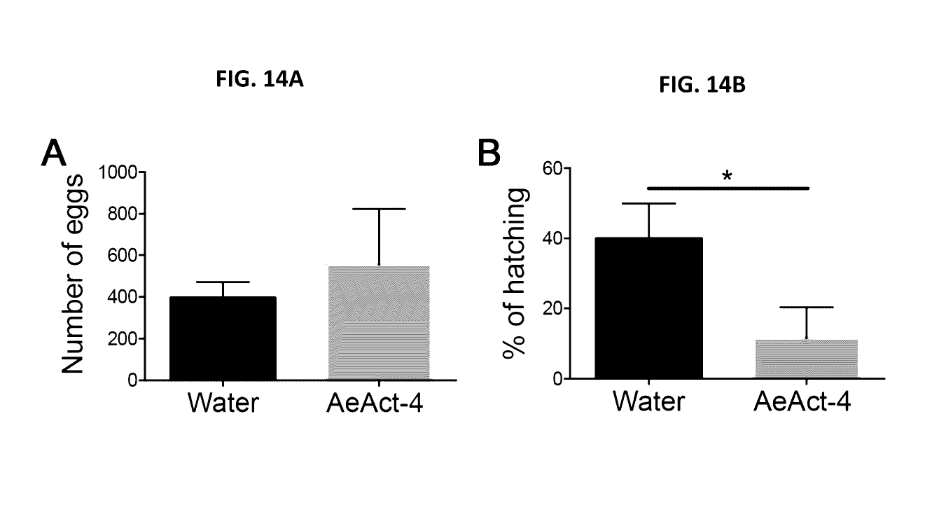

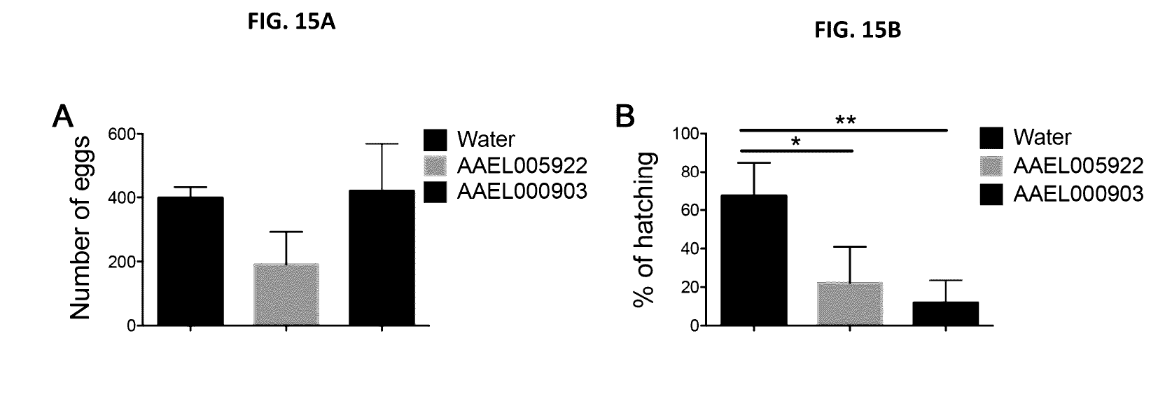

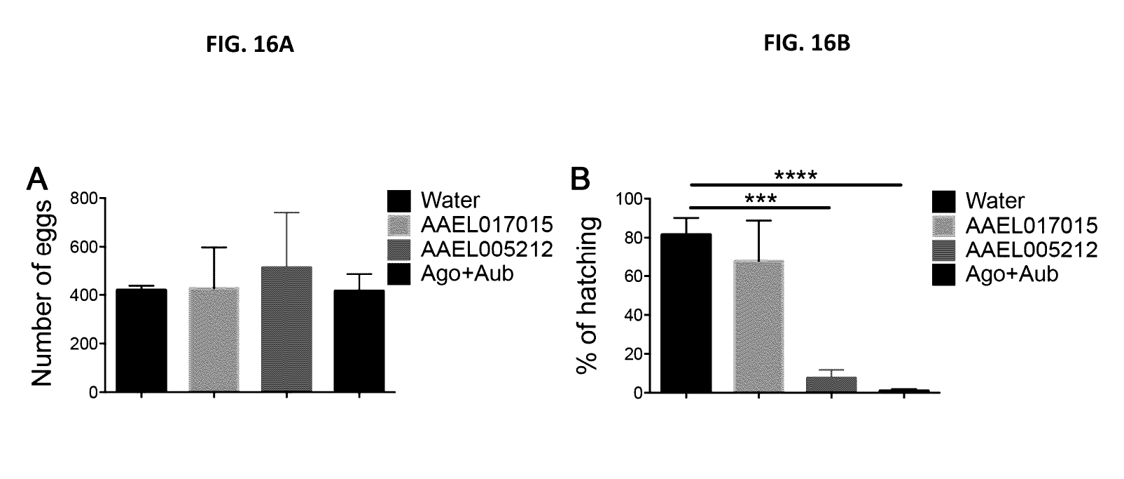

As illustrated in FIG. 10B, the present inventors were able to induce gene silencing in mosquito larvae after treatment with dsRNA against Ago3, one of the targets to induce male/female sterility. Next, larvae were treated with dsRNA against Aub and Ago3 and were reared until the adult stage. Female mosquitoes were allowed to blood fed on sheep blood and engorged females were separated in 3 cages containing 15 females each and the oviposition rate was calculated. As illustrated in FIGS. 13A-B, there was no difference in the oviposition rate among dsRNA-treated groups and water control. However, the number of hatched eggs decrease to 50% in the dsRNA treated groups. Similar results were observed for treatment with dsRNA targeting AY531222.2, AAEL005922, AAEL000903, AAEL017015 or AAEL005212 (see FIGS. 14A-B, 15A-B and 16A-B). When larvae were treated with dsRNA targeting the combination of Ago+Aub a much stronger reduction in the hatchability was observed (FIG. 16B).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A composition-of-matter for mosquito control, comprising a cell comprising an exogenous naked dsRNA which specifically down-regulates expression of a gene being endogenous to a mosquito or which specifically down-regulates expression of a gene being endogenous to a mosquito pathogen.

2. A composition-of-matter for mosquito control, comprising a cell comprising a nucleic acid larvicide.

3. A composition-of-matter for mosquito control, comprising a cell comprising a nucleic acid larvicide affecting fertility or fecundity of a mosquito.

4. A composition-of-matter for mosquito control comprising a nucleic acid larvicide that targets a piRNA pathway gene and/or a sterility gene.

5. (canceled)

6. The composition-of-matter of claim 4, wherein said nucleic acid larvicide comprises at least one dsRNA.

7. (canceled)

8. A method of producing a larvicidal composition, the method comprising introducing into a cell a nucleic acid larvicide, thereby producing the larvicide.

9. A method of producing a larvicidal composition, the method comprising introducing into a cell a nucleic acid larvicide affecting fertility or fecundity of a female mosquito, thereby producing the larvicide.

10-12. (canceled)

13. The composition-of-matter of claim 2, wherein said nucleic acid larvicide down-regulates a target gene selected from the group consisting of:
(i) affecting larval survival;
(ii) interfering with metamorphosis of larval stage to adulthood;
(iii) affecting susceptibility of mosquito larvae to a larvicide;
(iv) affecting susceptibility of an adult mosquito to an adulticide/insecticide; and
(v) affecting fertility or fecundity of a male or female mosquito.

14. The composition-of-matter of claim 13, wherein said target gene is selected from the group consisting of 1-427, 430-1813, 1826-1832.

15. The composition-of-matter of claim 13, wherein said target gene is selected from the group consisting of P-glycoprotein (AAEL010379), Argonaute-3 (AAEL007823), Cytochrome p450 (CYP9J26), Sodium channel (AAEL008297), Aub (AAEL007698), AeSCP-2 (AF510492.1), AeAct-4 (AY531222.2), AAEL002000, AAEL005747, AAEL005656, AAEL017015, AAEL005212, AAEL005922, AAEL000903 and AAEL005049.

16. The composition-of-matter of claim 13, wherein said target gene comprises Aub (AAEL007698) and Argonaute-3 (AAEL007823).

17. The composition-of-matter of claim 16, wherein said nucleic acid larvicide which down-regulates said target gene is a dsRNA.

18. The composition-of-matter of claim 17, wherein said dsRNA comprises SEQ ID NOs: 1858 and 1823.

19. The composition-of-matter of claim 3, wherein said cell is an algal cell.

20. The composition-of-matter of claim 3, wherein said cell is a microbial cell.

21. The composition-of-matter of claim 20, wherein said cell is a bacterial cell.

22. The composition-of-matter of claim 3, wherein the composition further comprises a food-bait.

23. The composition-of-matter of claim 3, wherein the composition is formulated in a formulation selected from the group consisting of technical powder, wettable powder, dust, pellet, briquette, tablet and granule.

24-25. (canceled)

26. The composition-of-matter of claim 3, wherein the composition is formulated as a semi-solid form.

27. The composition-of-matter of claim 26, wherein said semi-solid form comprises an agarose.

28. The composition-of-matter of claim 3, wherein the cell is lyophilized.

29. The composition-of-matter of claim 3, wherein the cell is non-transgenic.

30. (canceled)

31. The composition-of-matter of claim 3, wherein said nucleic acid larvicide comprises a dsRNA.

32. The composition-of-matter or method of claim 31, wherein said dsRNA is a naked dsRNA.

33-34. (canceled)

35. The composition-of-matter of claim 31, wherein said dsRNA is effected at a dose of 0.001-1 μg/μL for soaking or at a dose of 1 pg to 10 μg/larvae for feeding.

36. The composition-of-matter of claim 31, wherein said dsRNA is selected from the group consisting of SEQ ID NOs: 1822-1825 and 1857-1868.

37-43. (canceled)

44. The composition-of-matter of claim 1 having an inferior impact on an adult mosquito as compared to said larvae.

45. (canceled)

46. The composition-of-matter of claim 1, further comprising a larvicide and wherein said larvicide is selected from the group consisting of Temephos, Diflubenzuron, methoprene, Bacillus sphaericus, and Bacillus thuringiensis israelensis.

47. The composition-of-matter of claim 1, further comprising a larvicide and wherein said larvicide comprises an adulticide.

48. The composition-of-matter of claim 47, wherein said adulticide is selected from the group consisting of deltamethrin, malathion, naled, chlorpyrifos, permethrin, resmethrin and sumithrin.

49. A method of controlling or exterminating mosquitoes, the method comprising feeding larvae of the mosquitoes with an effective amount of the composition-of-matter of claim 3, thereby controlling or exterminating the mosquitoes.

50. The method of claim 49, wherein said mosquitoes comprise female mosquitoes capable of transmitting a disease to a mammalian organism.

51. The method of claim 49, wherein said mosquitoes are of a species selected from the group consisting of Aedes aegypti and Anopheles gambiae.

52. The composition-of-matter of claim 2, wherein said cell is an algal cell.

53. The composition-of-matter of claim 3, wherein said cell is a microbial cell.

54. The composition-of-matter of claim 2, wherein said cell is an algal cell.

55. The composition-of-matter of claim 3, wherein said cell is a microbial cell.

56. The composition-of-matter of claim 2, wherein the cell is lyophilized.

57. The composition-of-matter of claim 3, wherein the cell is non-transgenic.

58. The composition-of-matter of claim 2, wherein the cell is lyophilized.

59. The composition-of-matter of claim 3, wherein the cell is non-transgenic.
